(19) United States
(12) Patent Application Publication
(10) Pub. No.: US 2024/0041931 A1
(43) Pub. Date: Feb. 8, 2024

(54) COMPOSITIONS AND METHODS FOR TREATING CANCER

(71) Applicants: UNIVERSITY OF PITTSBURGH-OF THE COMMONWEALTH SYSTEM OF HIGHER EDUCATION, Pittsburgh, PA (US); THE UNITED STATES OF AMERICA, AS REPRESENTED BY THE SECRETARY, DEPARTMENT OF HEALTH AND HUMAN SERVIC, Bethesda, MD (US)

(72) Inventors: Diwakar DAVAR, Pittsburgh, PA (US); Hassane Mohamed ZAROUR, Pittsburgh, PA (US); Giorgio TRINCHIERI, Bethesda, MD (US); Amiran Kasanovich DZUTSEV, Bethesda, MD (US)

(21) Appl. No.: 18/266,455

(22) PCT Filed: Dec. 10, 2021
(86) PCT No.: PCT/US2021/062832

§ 371 Date: Jun. 9, 2023

Related U.S. Application Data
(60) Provisional application No. 63/124,231, filed on Dec. 11, 2020.

(51) Int. Cl. A61K 35/24; A61K 39/395; A61K 35/741; A61P 35/00

(52) U.S. Cl. CPC A61K 35/24; A61K 39/3955; A61K 35/741; A61P 35/00; A61K 2035/115

(57) ABSTRACT

Disclosed herein are methods for treating cancer by administering an anti-PD-1 antibody and a fecal sample obtained from a donor that is responsive to an anti-PD-1 therapy. Specifically, the cancer comprising a melanoma, and a metastatic melanoma; and wherein the fecal sample comprises a higher level of bacteria of phylum Actinobacteria and/or phylum Firmicutes in comparison to a control.

### CROSS REFERENCE TO RELATED APPLICATIONS

This application claims the priority benefit of U.S. Provisional Application No. 63/124,231, filed Dec. 11, 2020, which is expressly incorporated herein by reference.

### STATEMENT REGARDING FEDERALLY SPONSORED RESEARCH OR DEVELOPMENT

This invention was made with government support under grant number CA222203 awarded by the National Institutes of Health. The government has certain rights in the invention.

### BACKGROUND OF THE INVENTION

Anti-programmed cell death protein 1 (PD-1) therapy provides long-term clinical benefits to patients with advanced melanoma. However, many patients do not respond sufficiently to the treatment. What is needed are compositions and methods for overcoming the resistance to anti-PD-1 therapy. The compositions and methods disclosed herein address these and other needs.

### SUMMARY OF THE INVENTION

Disclosed herein are methods for treating a cancer in a subject comprising administering to the subject a therapeutically effective amount of a fecal sample and an anti-PD-1 antibody, wherein the fecal sample is derived from a donor that is responsive to an anti-PD-1 antibody. In some examples, the subject is less responsive to the anti-PD-1 antibody than the donor or non-responsive to the anti-PD-1 antibody. In some embodiments, the anti-PD-1 antibody administered to the subject is different from the anti-PD-1 antibody received by the donor.

In some examples, the fecal samples provided herein have a higher level of bacteria of phylum Actinobacteria and/or phylum Firmicutes in comparison to a control. Administration of the fecal sample can increase a level of bacteria of phylum Firmicutes and/or phylum Actinobacteria in the subject's gut in comparison to a control and/or decrease a level of bacteria of phylum Bacteroides in the subject's gut in comparison to a control.

The methods provided herein can be applied in combination with one or more administrations of an anti-PD-1 antibody. This method has been shown to be surprisingly effective at treating a cancer, including, for example, a melanoma, and in some embodiments, an anti-PD-1 refractory melanoma or an advanced anti-PD-1 refractory melanoma.

### BRIEF DESCRIPTION OF THE DRAWINGS

FIGS. 1A-1C show radiographic responses from a Phase II study of anti-PD-1 responder-derived FMT and pembrolizumab in anti-PD-1-refractory melanoma. Melanoma patients who had primary refractory disease to anti-PD-1 therapy received FMT derived from individual melanoma patients with durable objective response to anti-PD1 therapy. FMT was administered colonoscopically on day 0 along with pembrolizumab (200 mg). Pembrolizumab was repeated every 3 weeks. Restaging scans were performed at weeks 9-12 and repeated every 9-12 weeks while on study. Patients remained on study until intolerable toxicity, RECIST v1.1 confirmed disease progression, or completion of 35 cycles of pembrolizumab. FIGS. 1A and 1B show treatment exposure and response duration by RECIST v1.1 (investigator assessed; n=15). In FIG. 1A, FMT donor and best response to prior line(s) of anti-PD(L)1 therapy singly or in combination are shown for each FMT recipient patient. Length of each bar corresponds to duration of time patients received treatment (in weeks). Response status is color coded (R—blue; NR—red). Response symbols represent status at first restaging scan (9-12 weeks) and at most recent review. Patients with ongoing response on study are depicted with horizontal arrows. FIG. 1B shows radiographic change of tumor burden from baseline (investigator assessed per RECIST v1.1; n=15). One patient (PT-18-0018) had initial disease stability with subsequent progression following antibiotic therapy and was offered a re-transplant with the same donor, with subsequent disease stabilization. FIG. 1C shows representative CT scans from one responding patient. CT scans from patient PT-19-0024 at 4 separate timepoints depict initial tumor growth after FMT followed by eventual PR.

FIGS. 2A-2F show microbiome analyses pre- and post-FMT in melanoma patients. FIG. 2A shows dimensionality reduction using t-distributed uniform manifold approximation and projection (t-UMAP) plot of microbial taxa abundances by last known taxon (LKT) of FMT recipients at different time points. Each color corresponds to a different FMT treated patient.

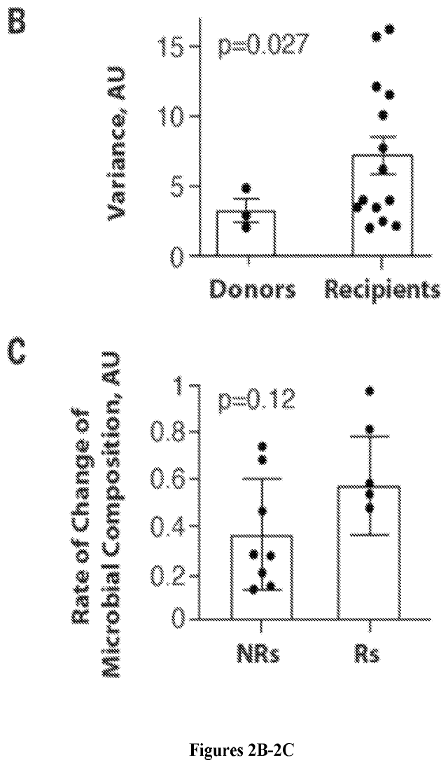

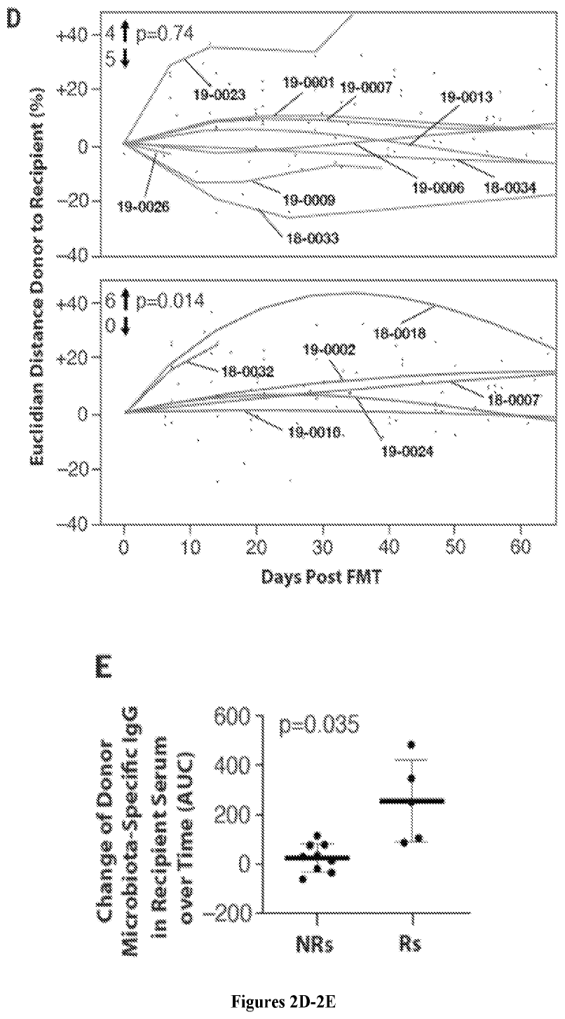

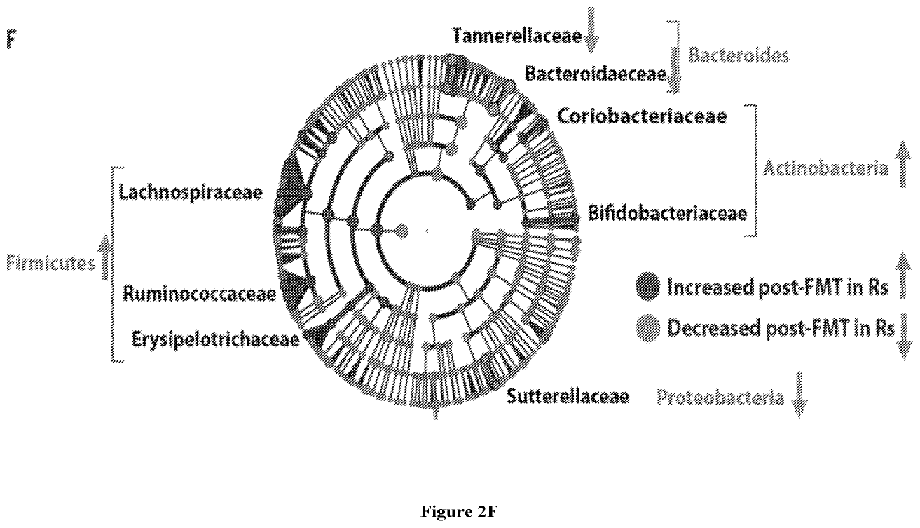

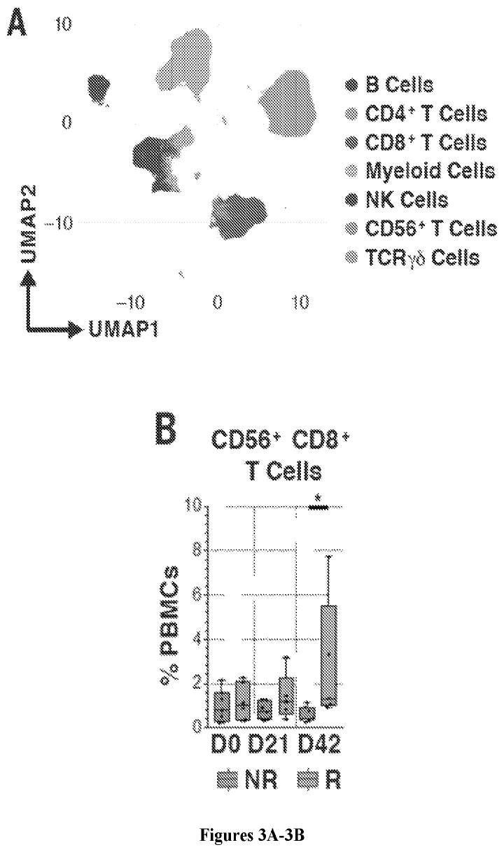

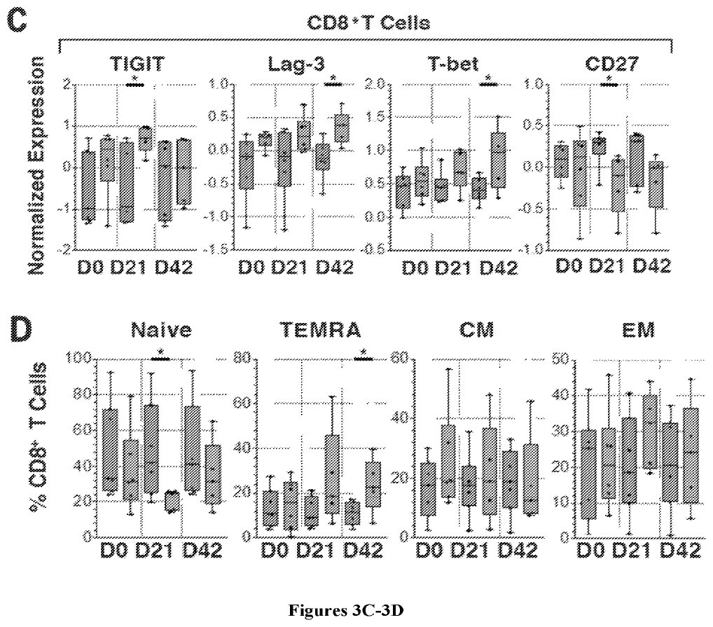

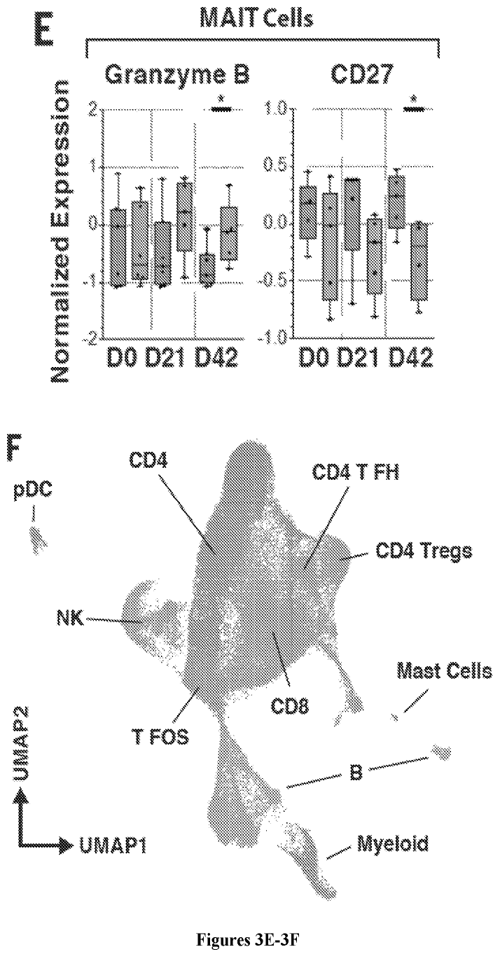

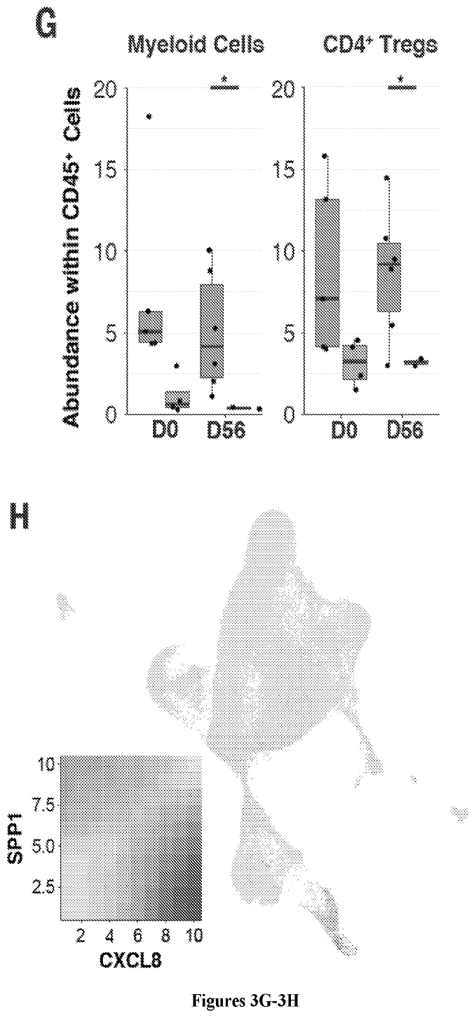

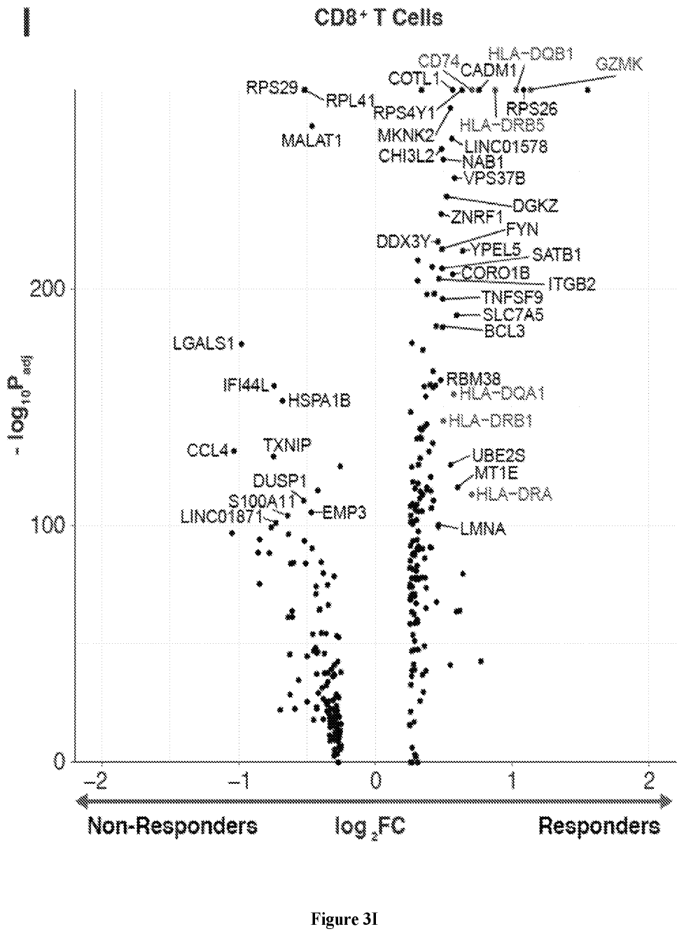

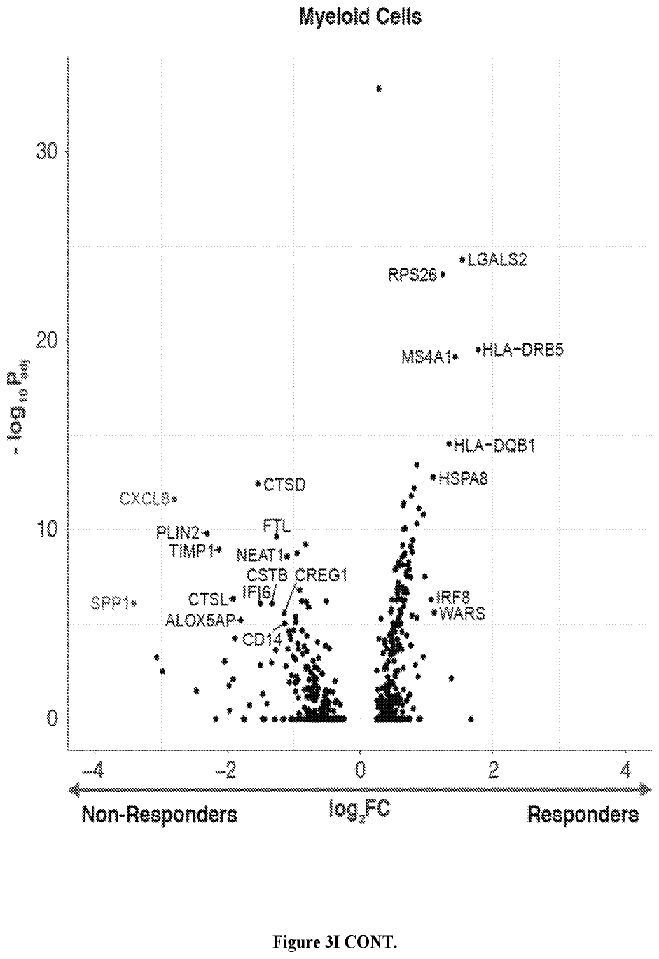

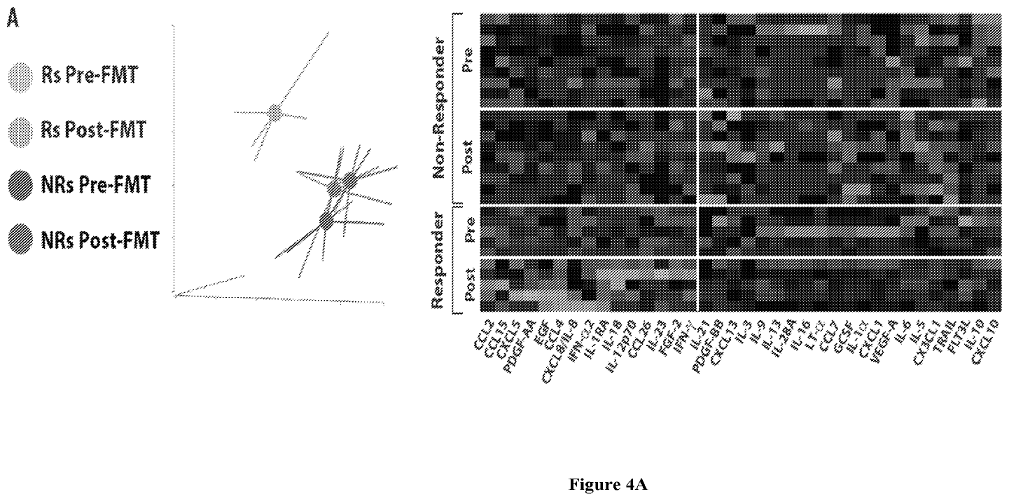

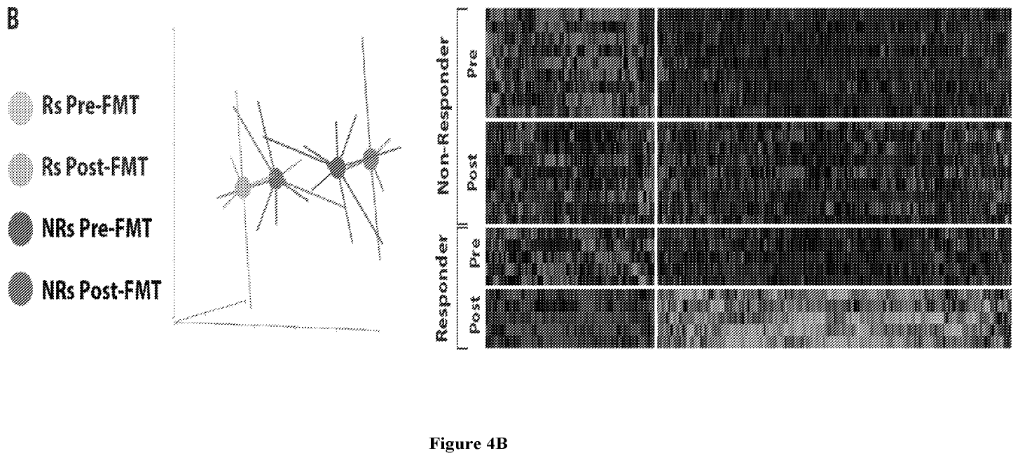

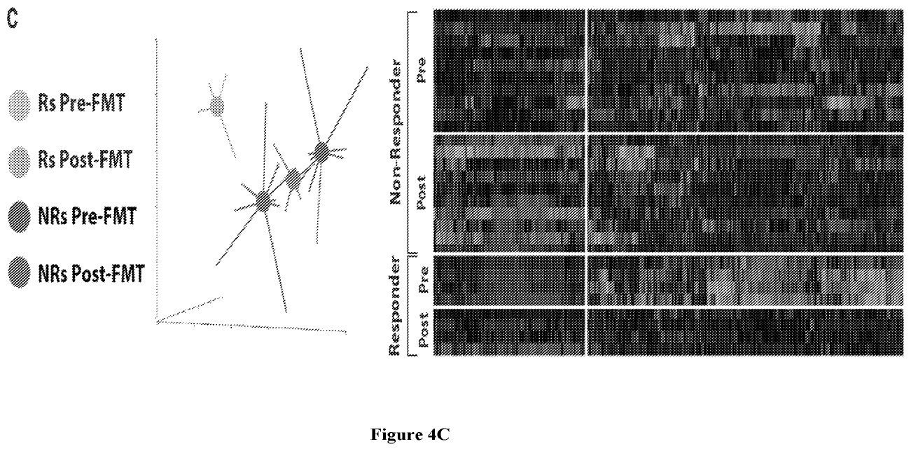

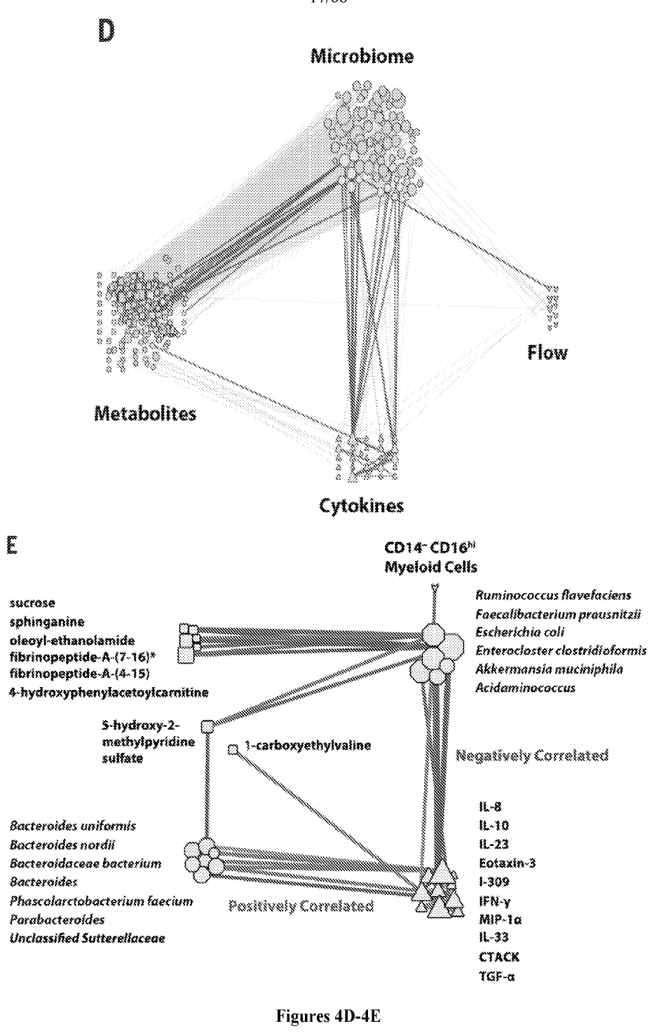

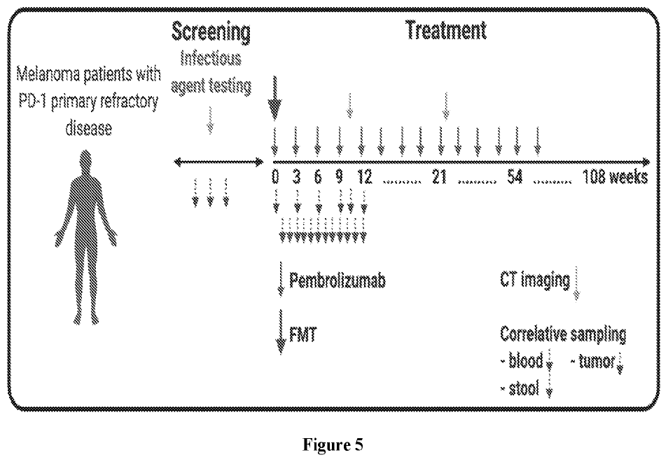

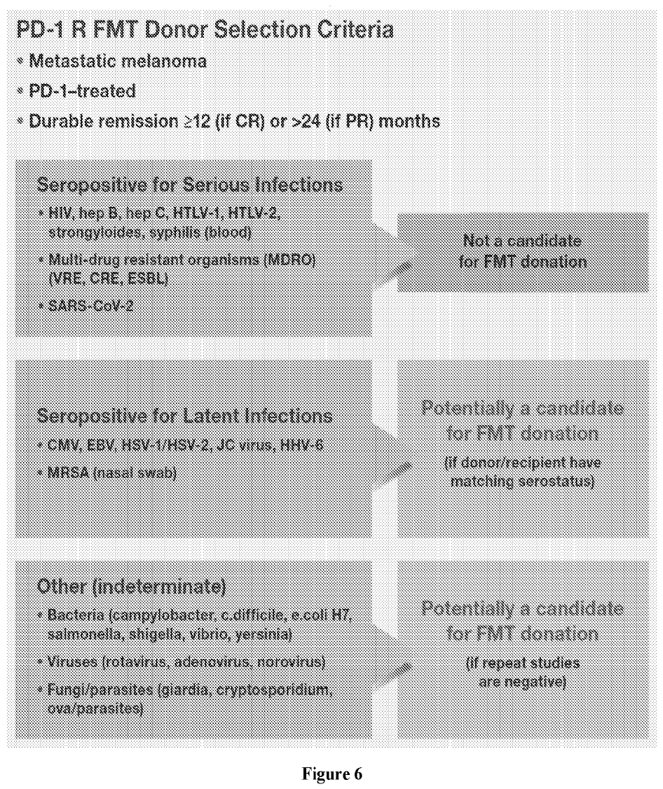

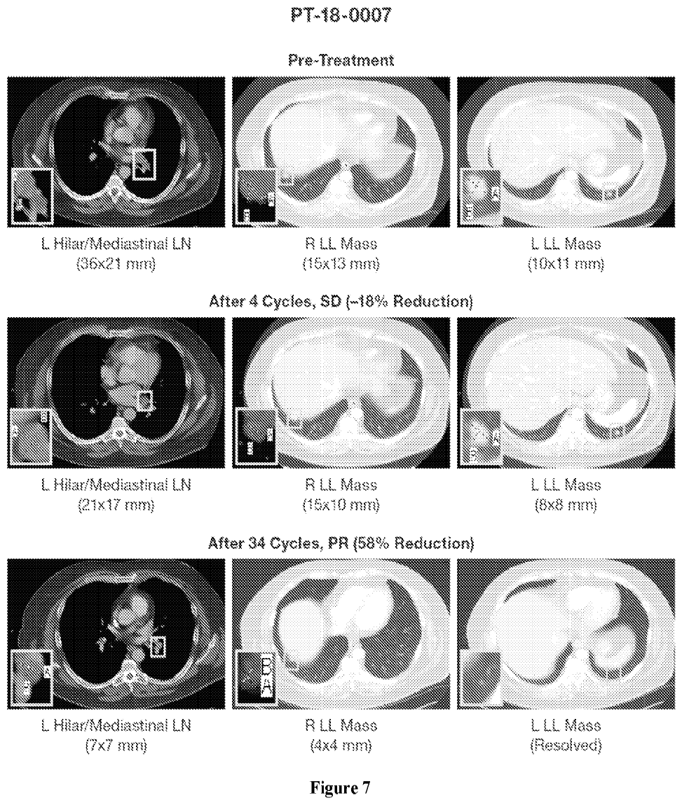

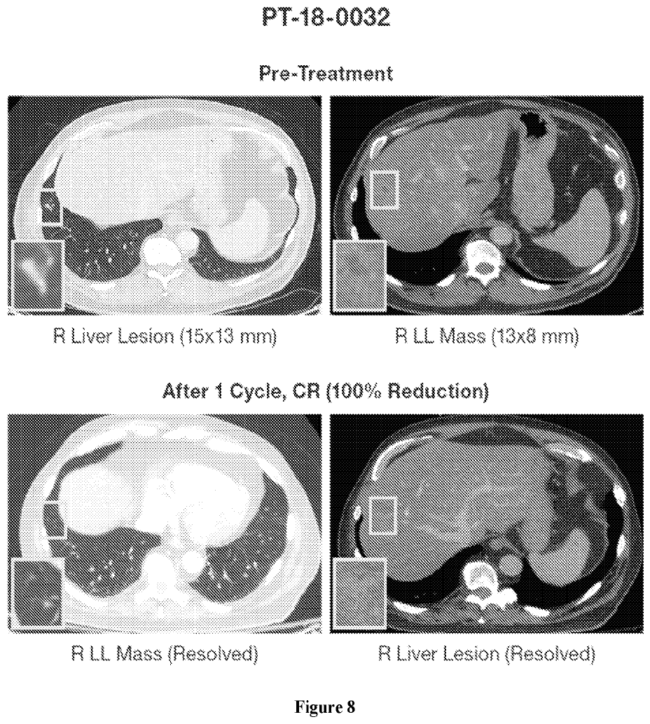

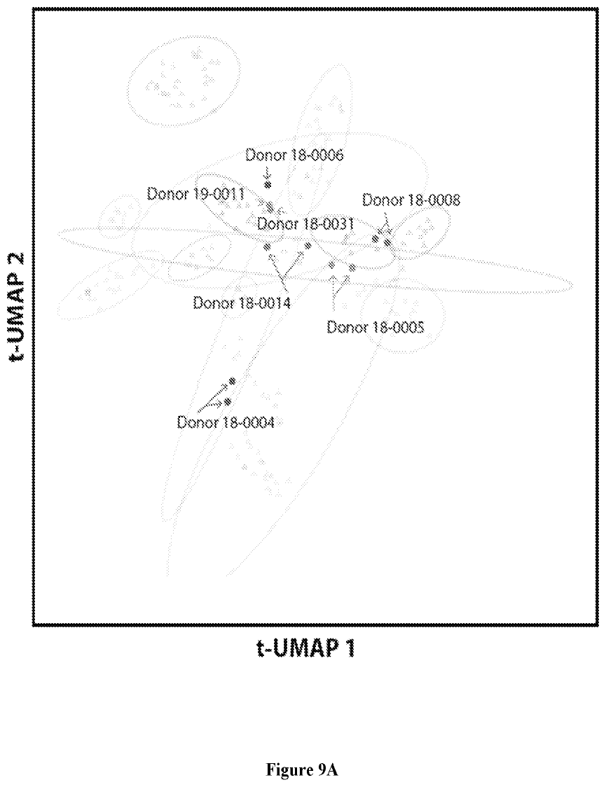

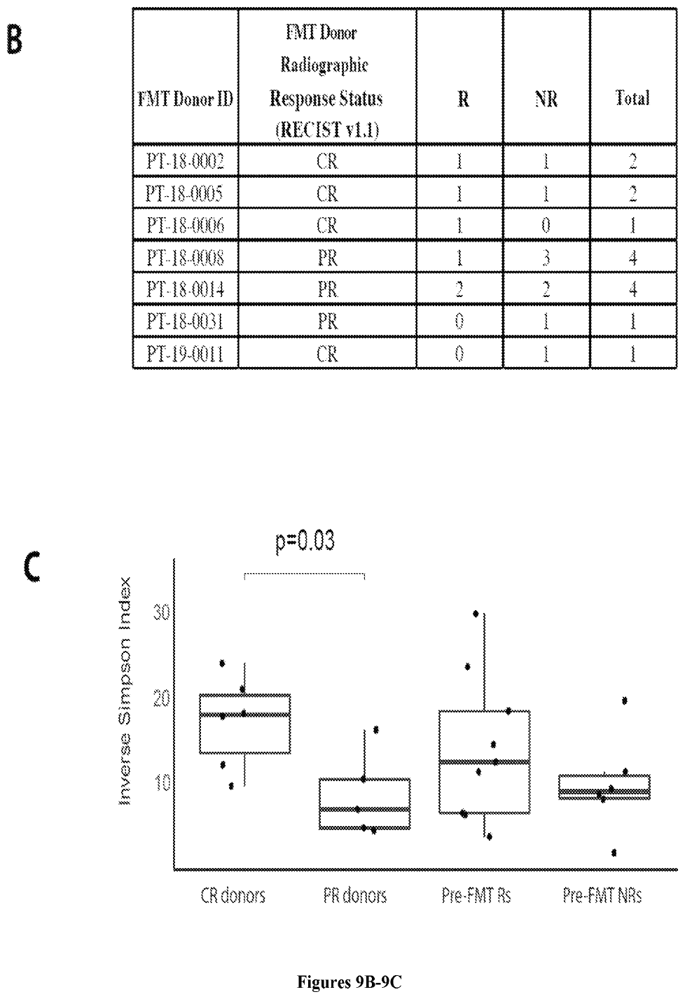

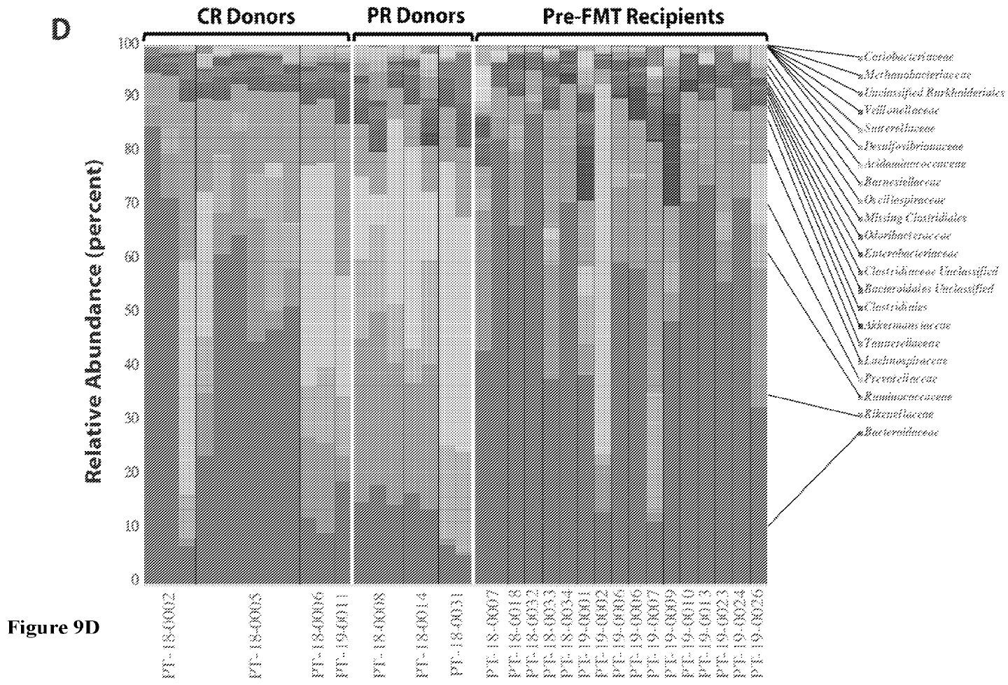

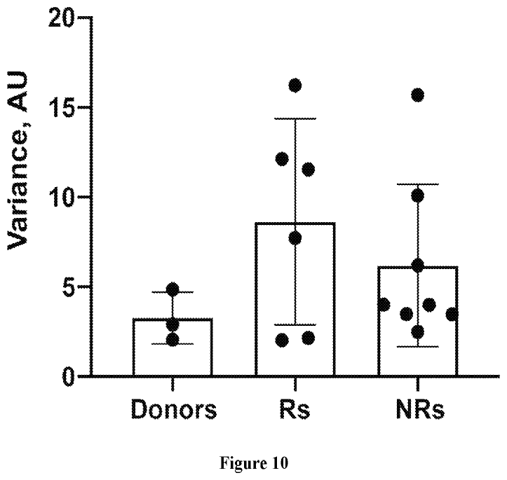

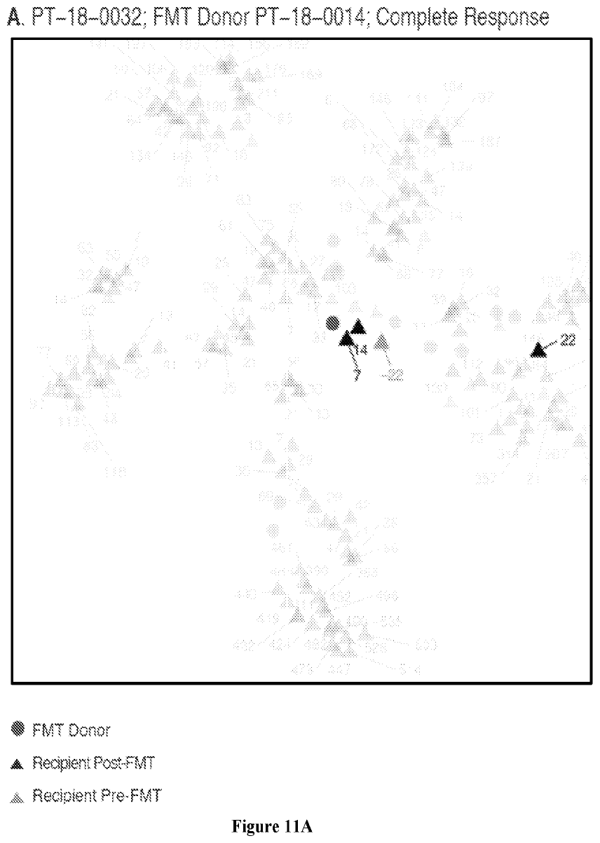

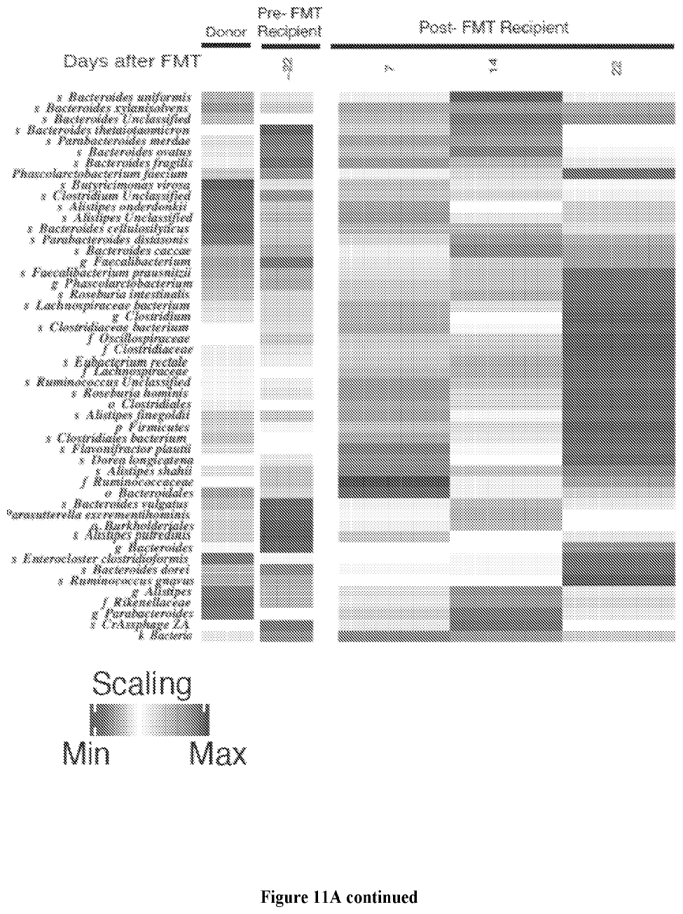

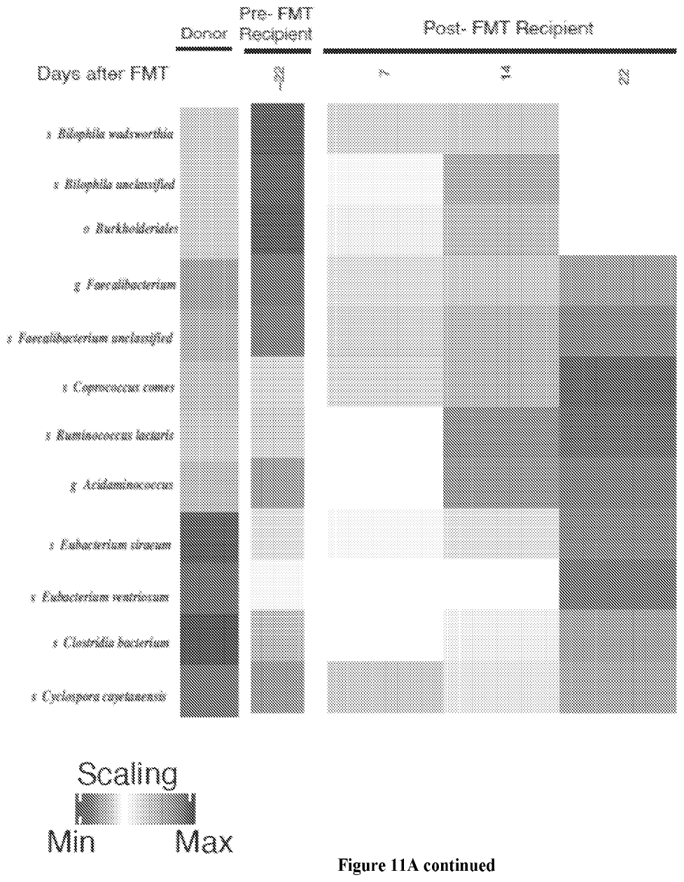

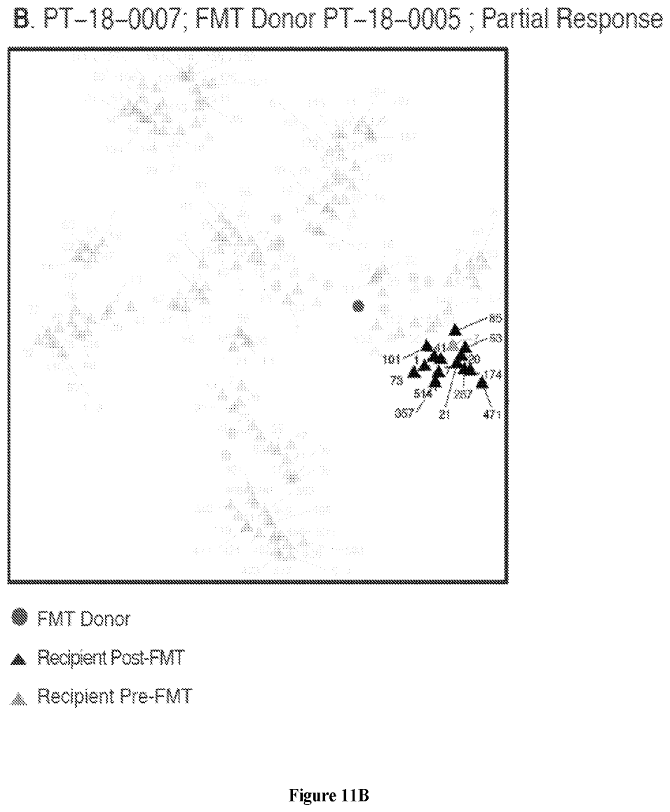

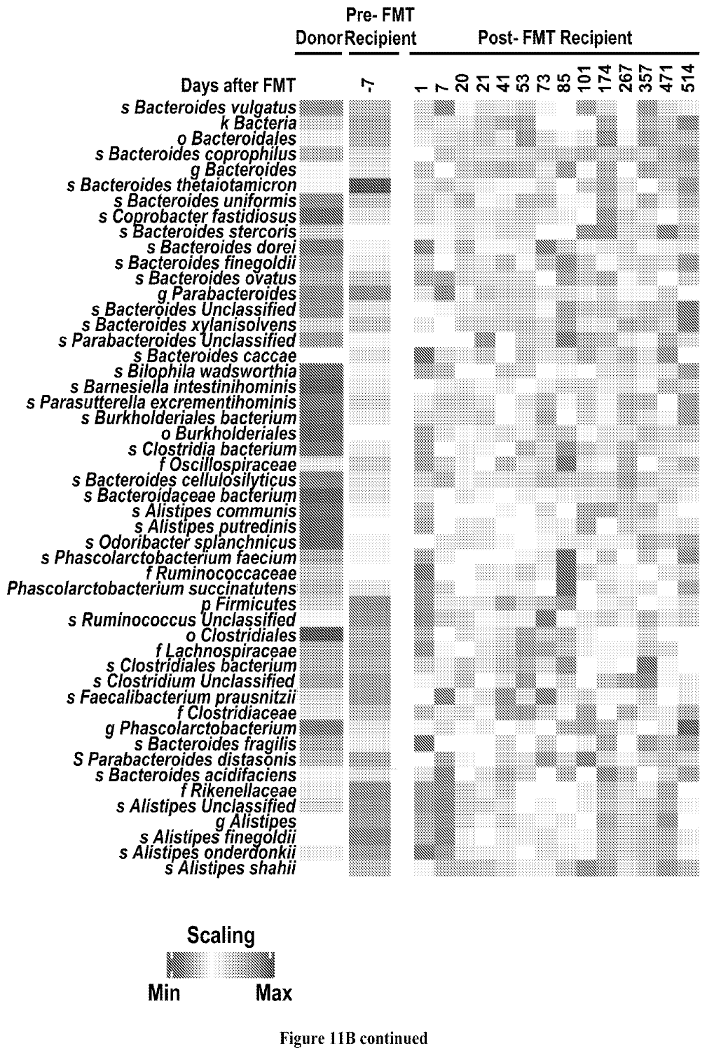

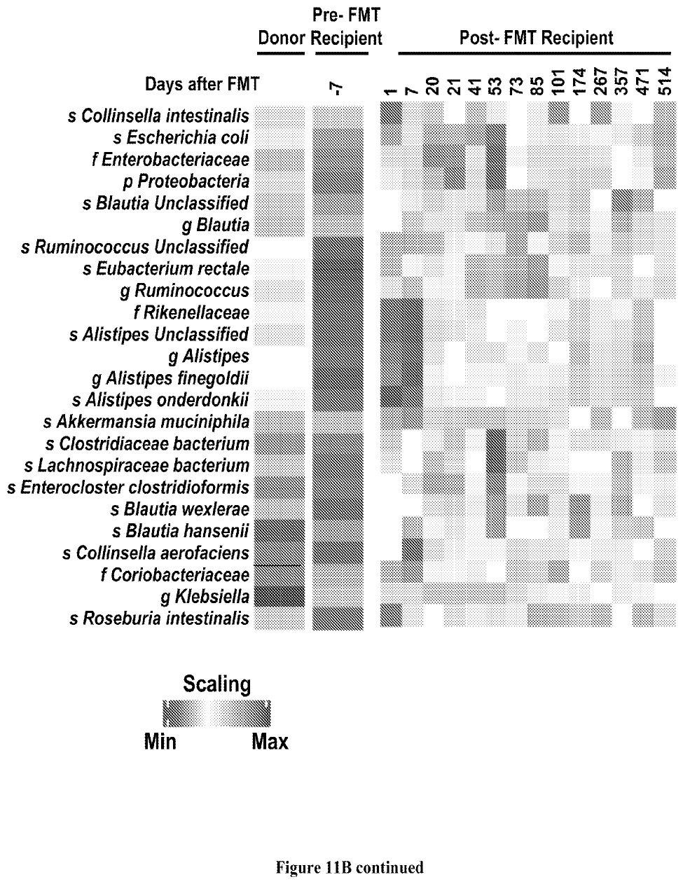

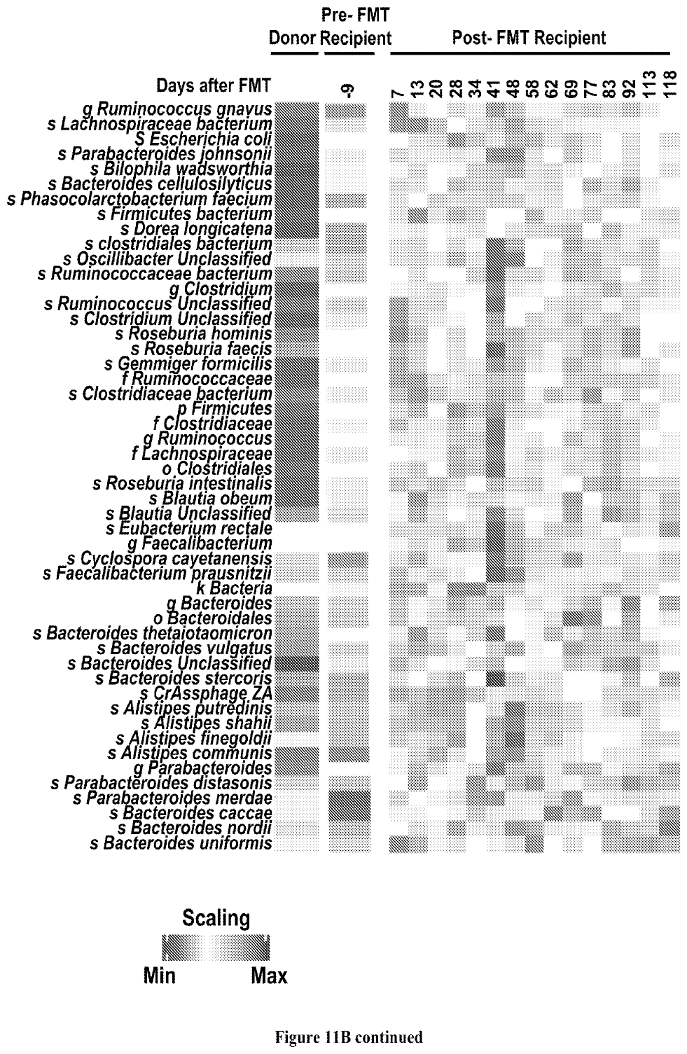

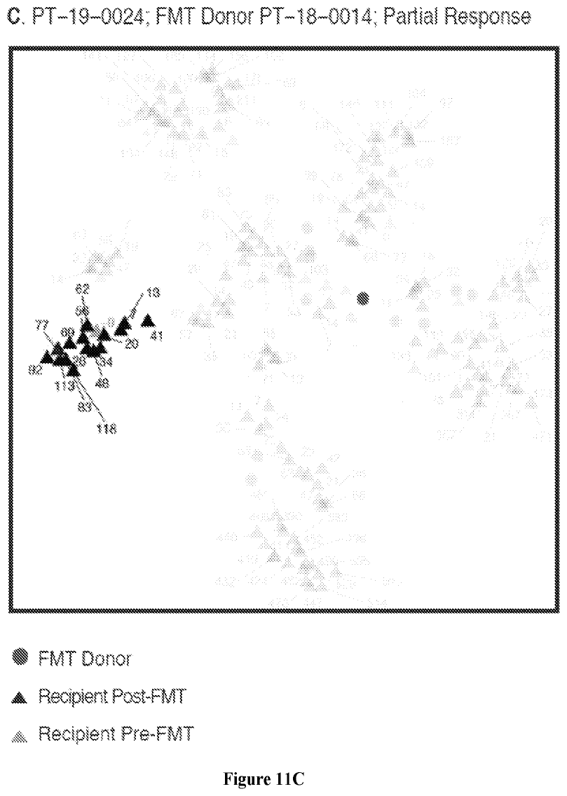

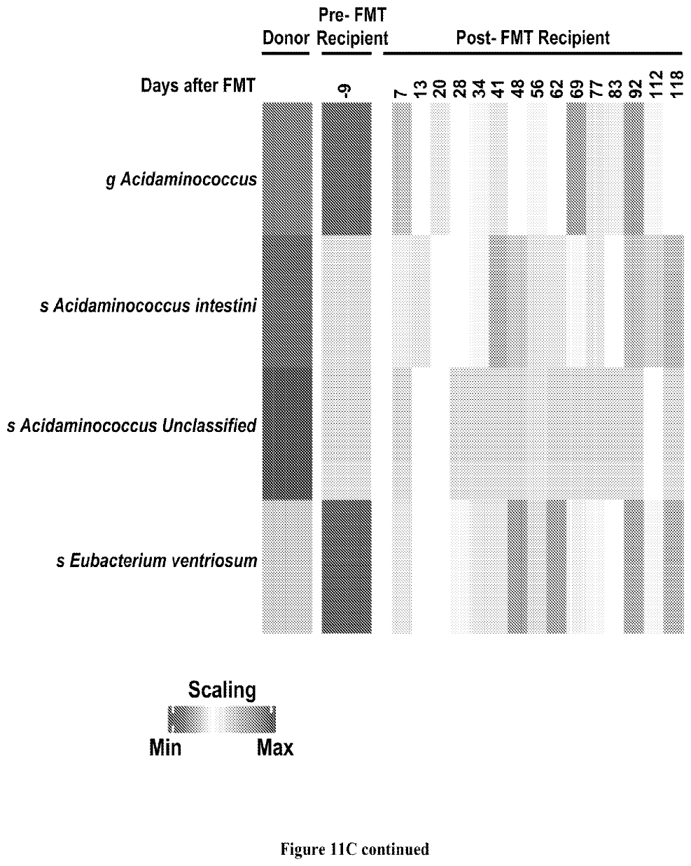

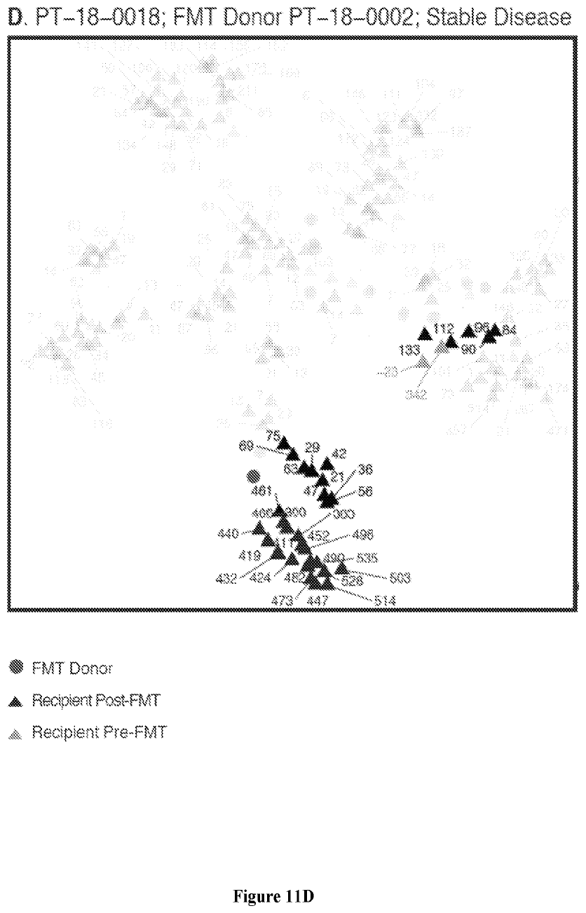

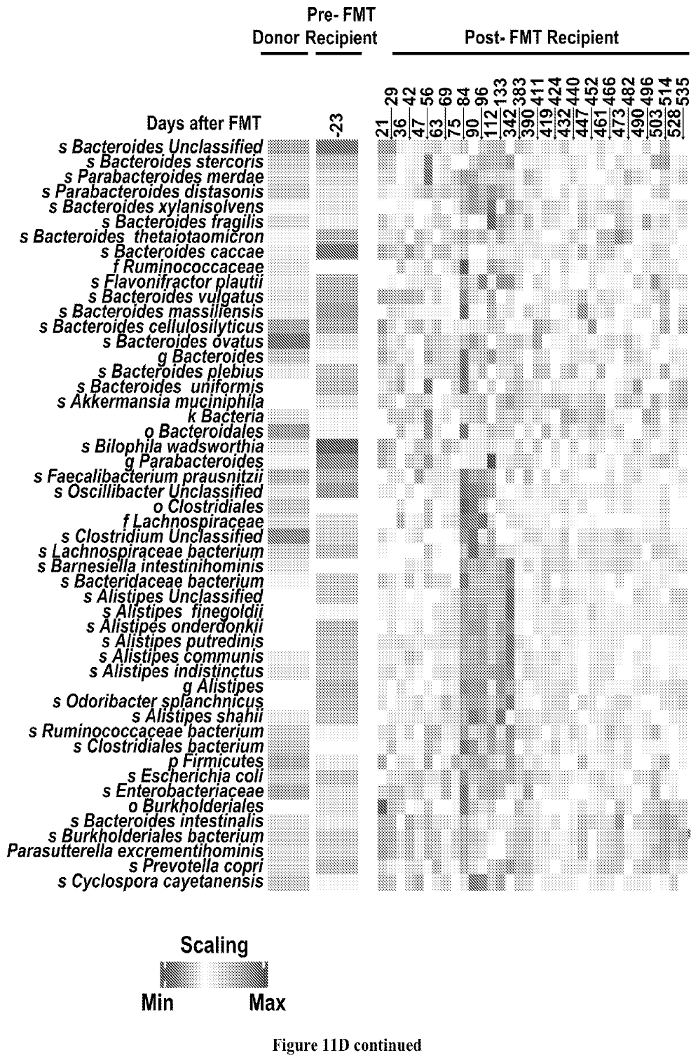

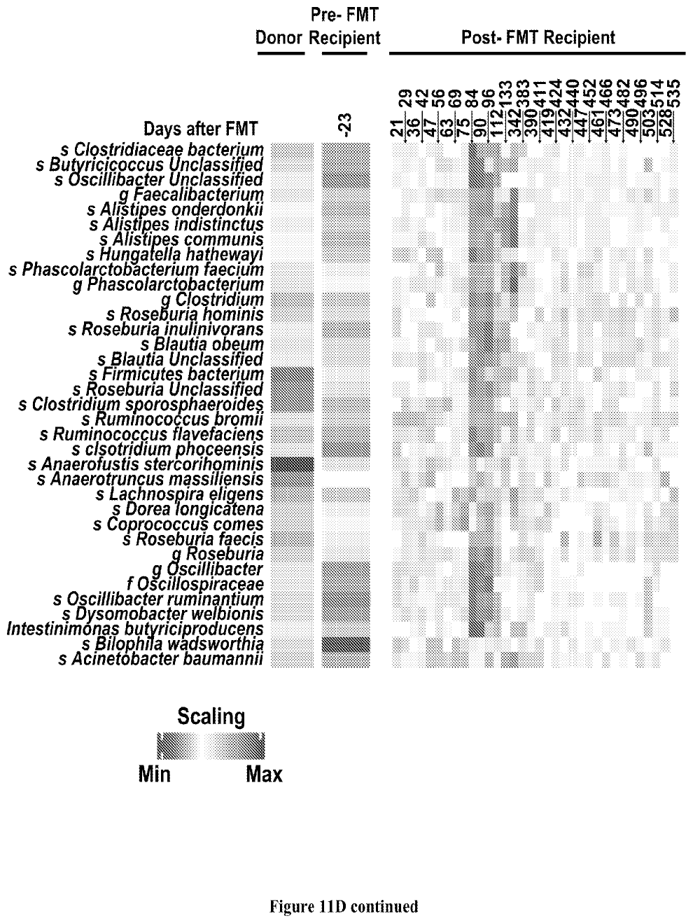

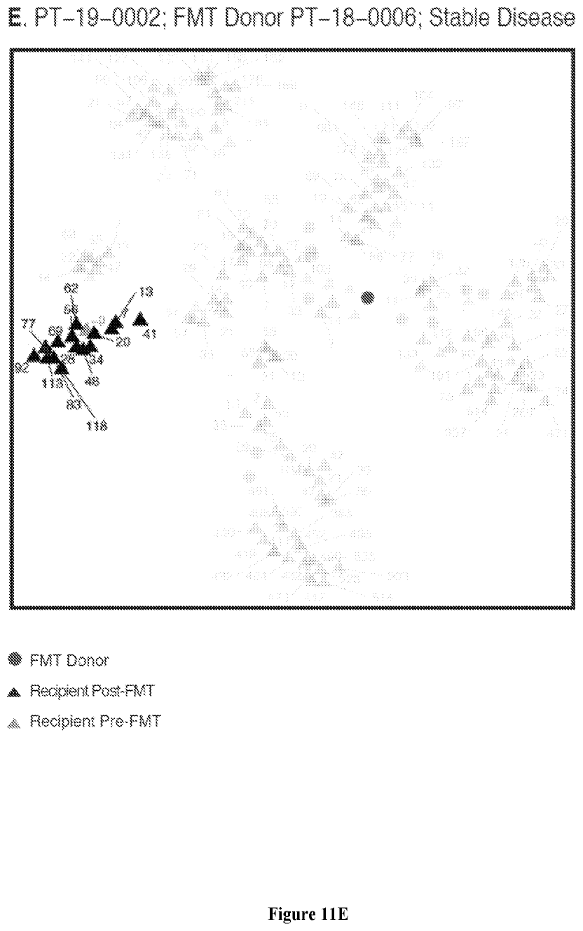

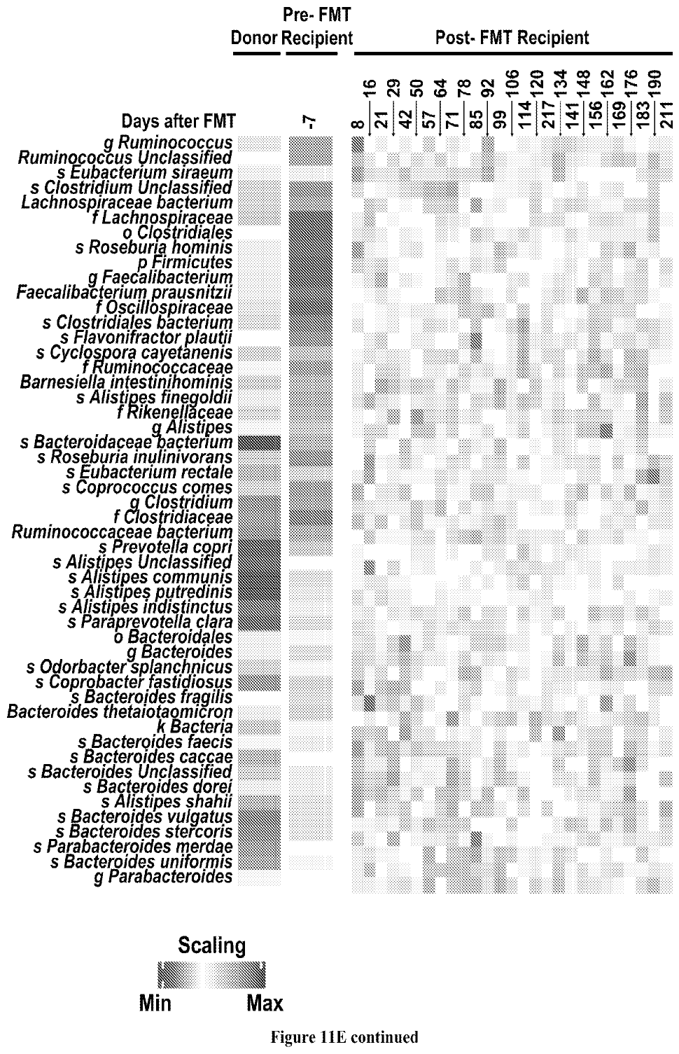

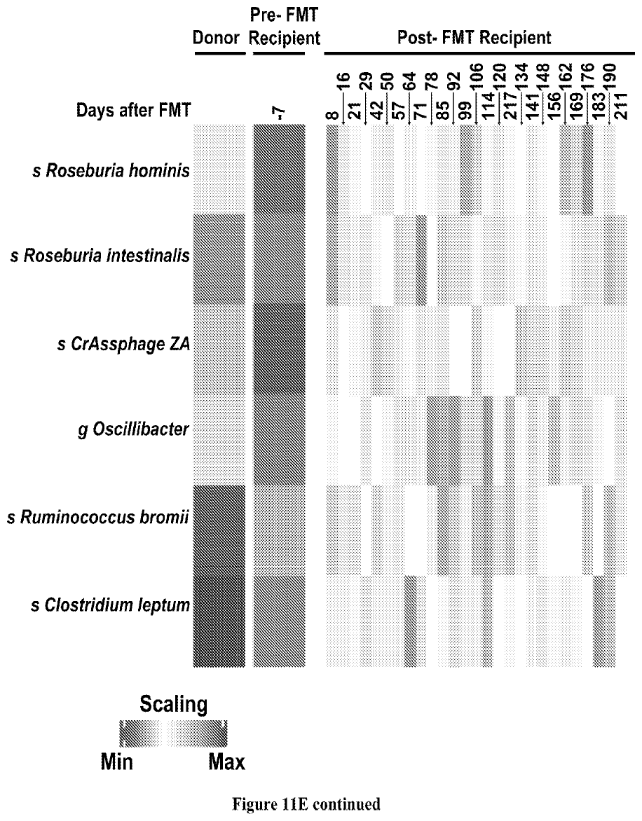

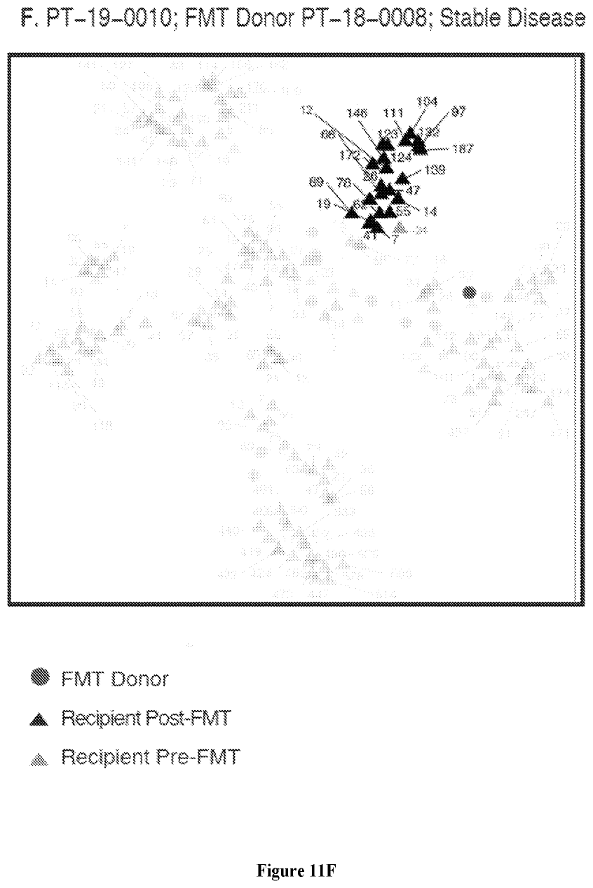

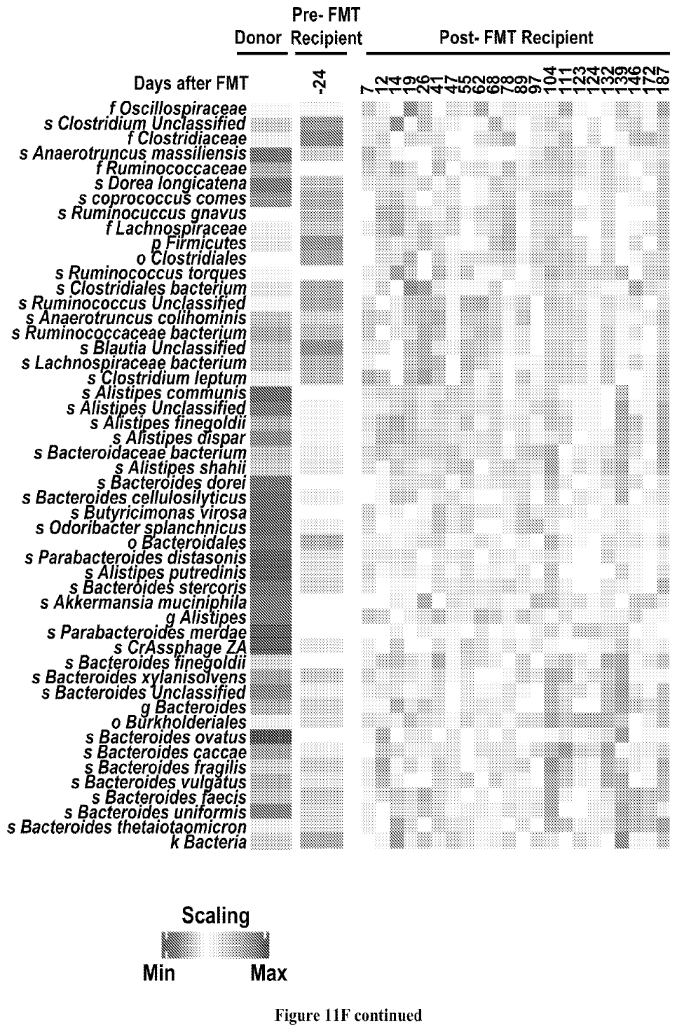

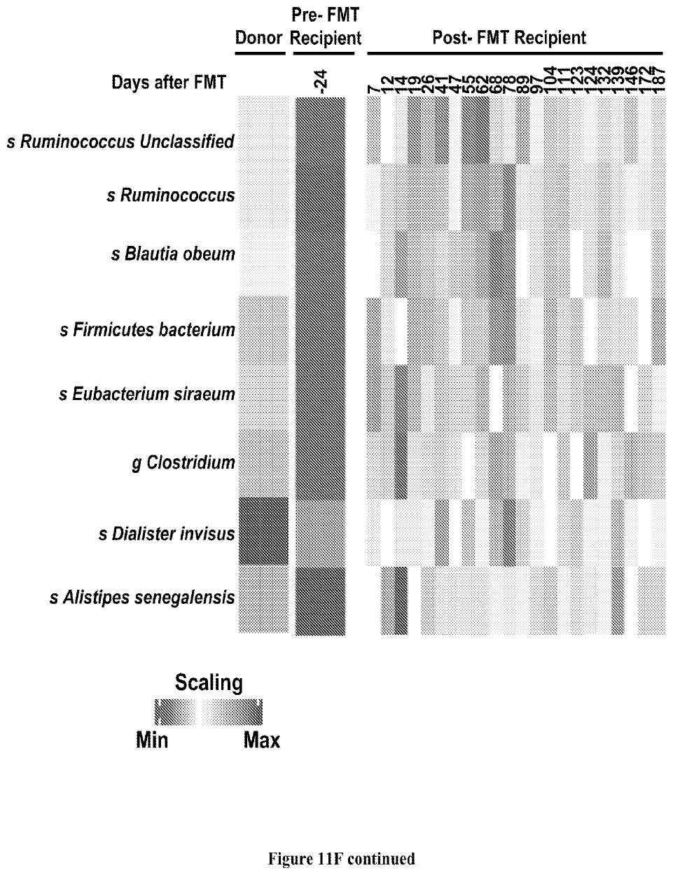

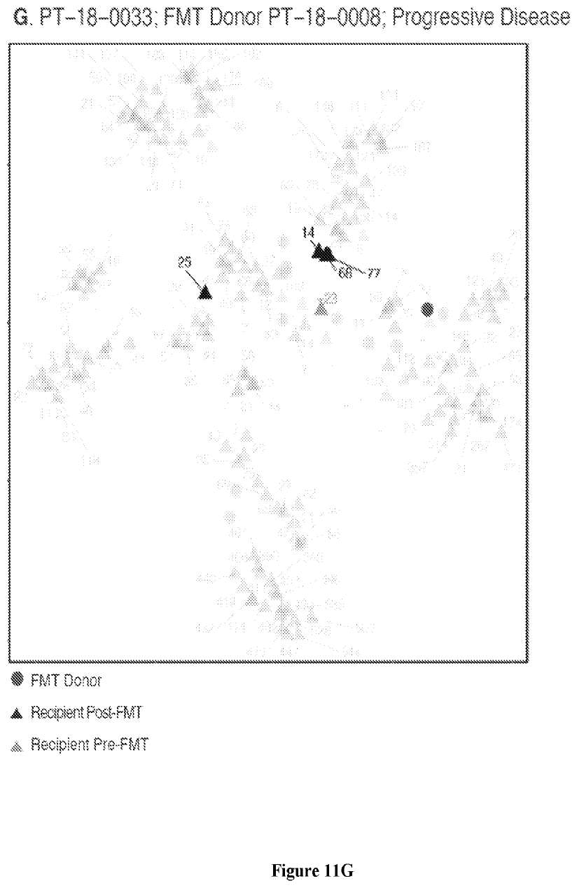

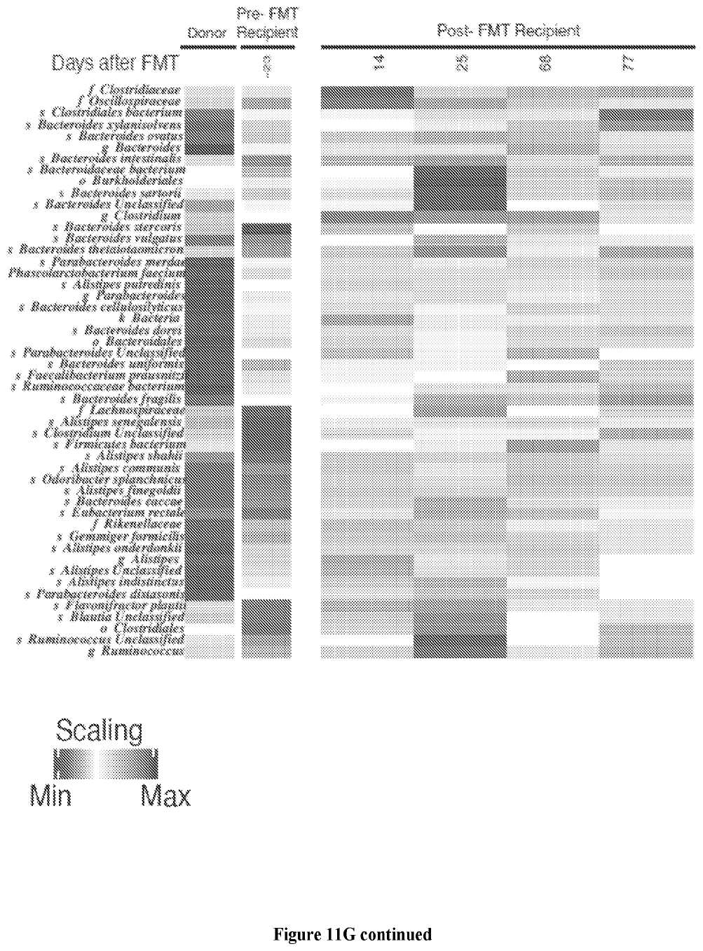

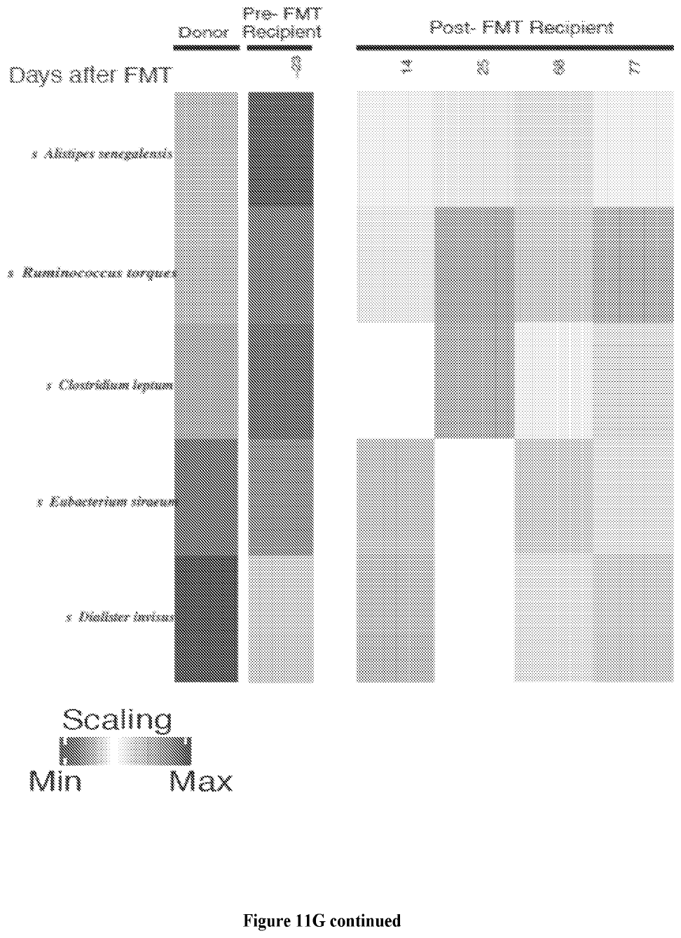

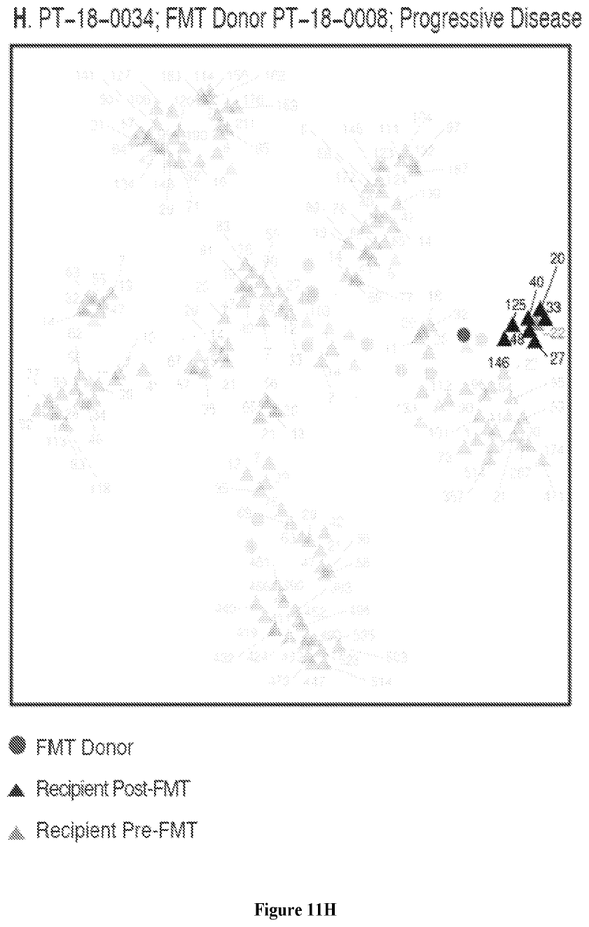

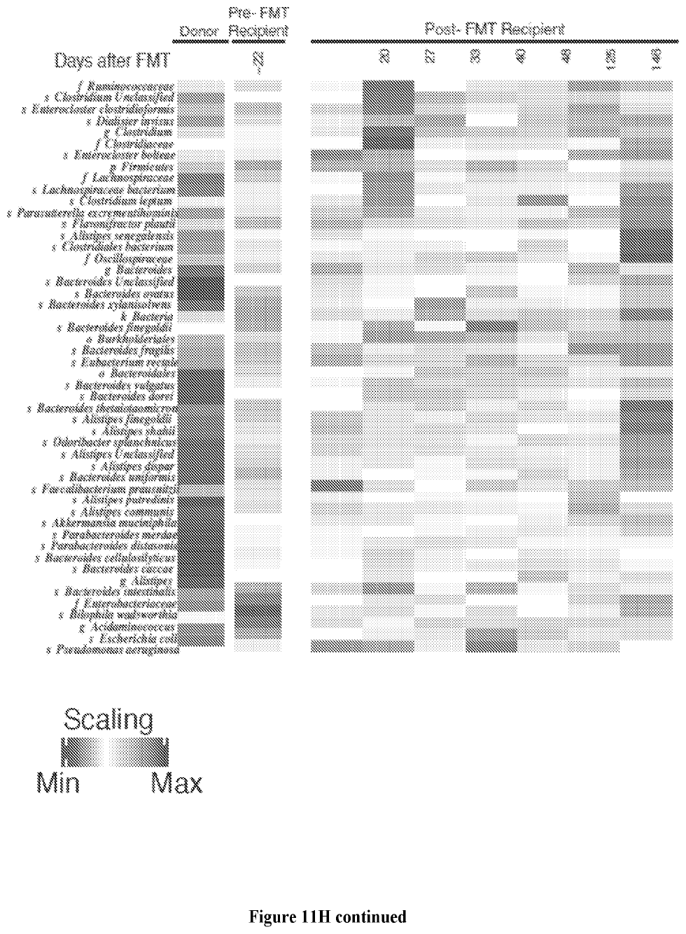

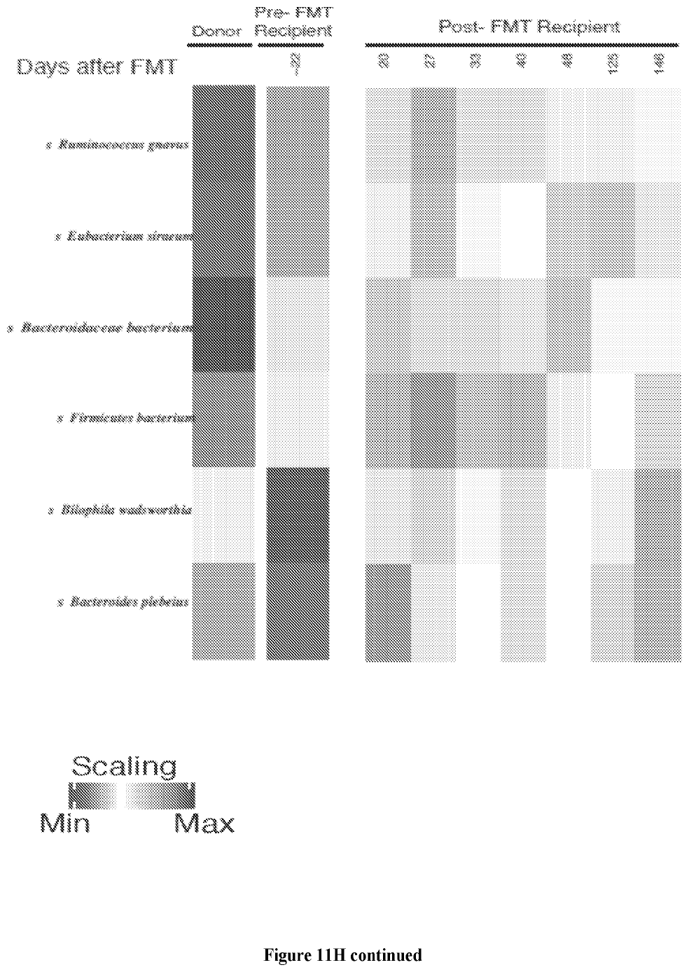

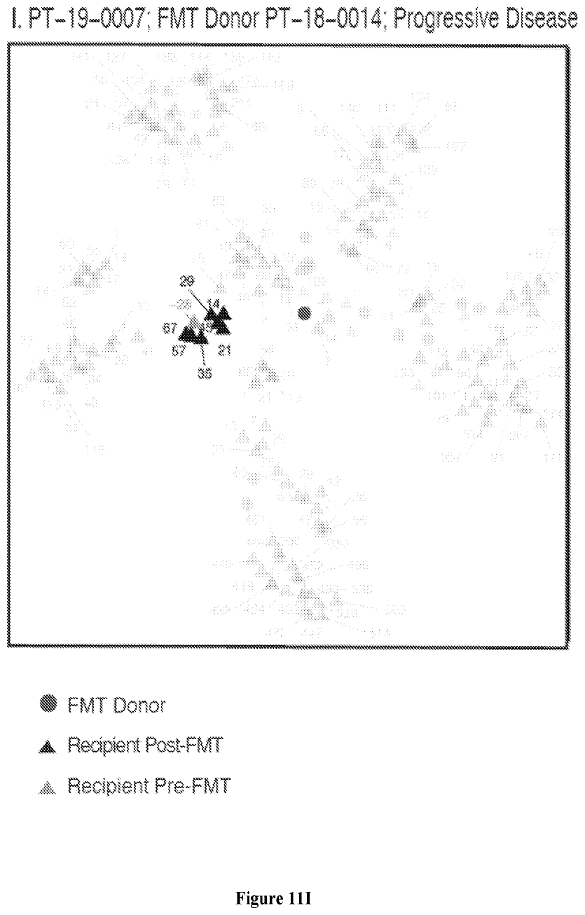

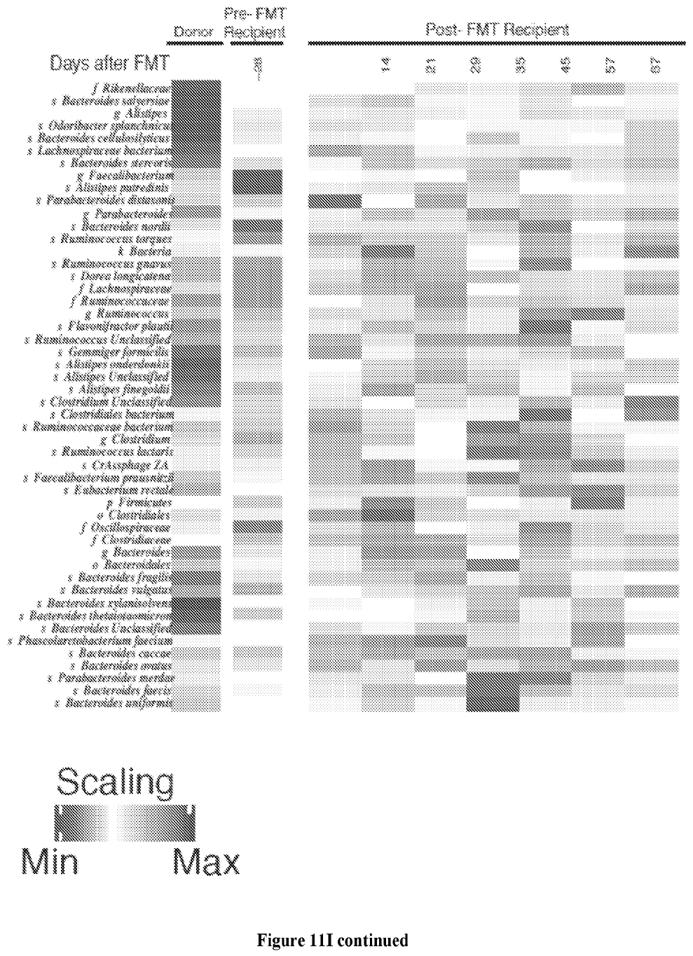

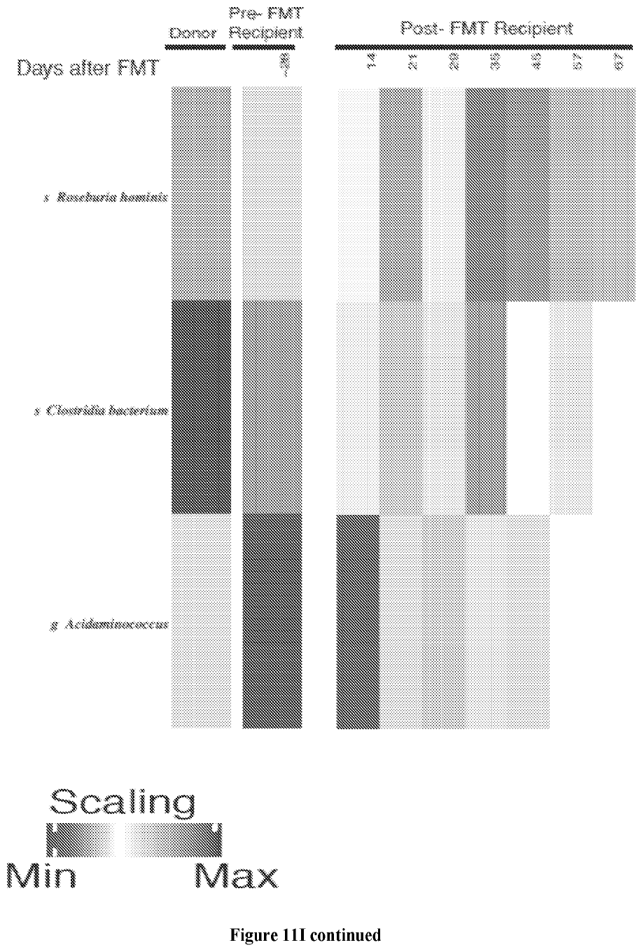

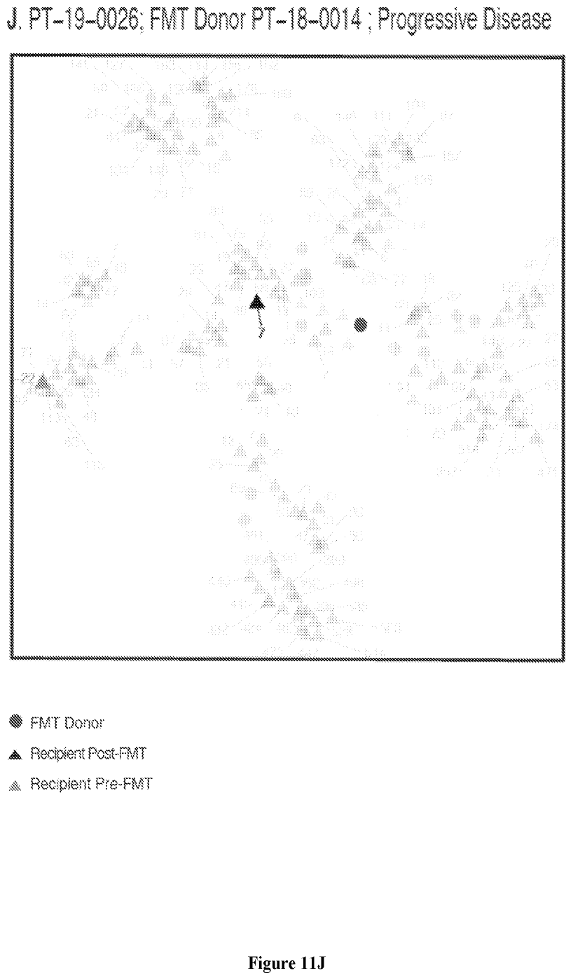

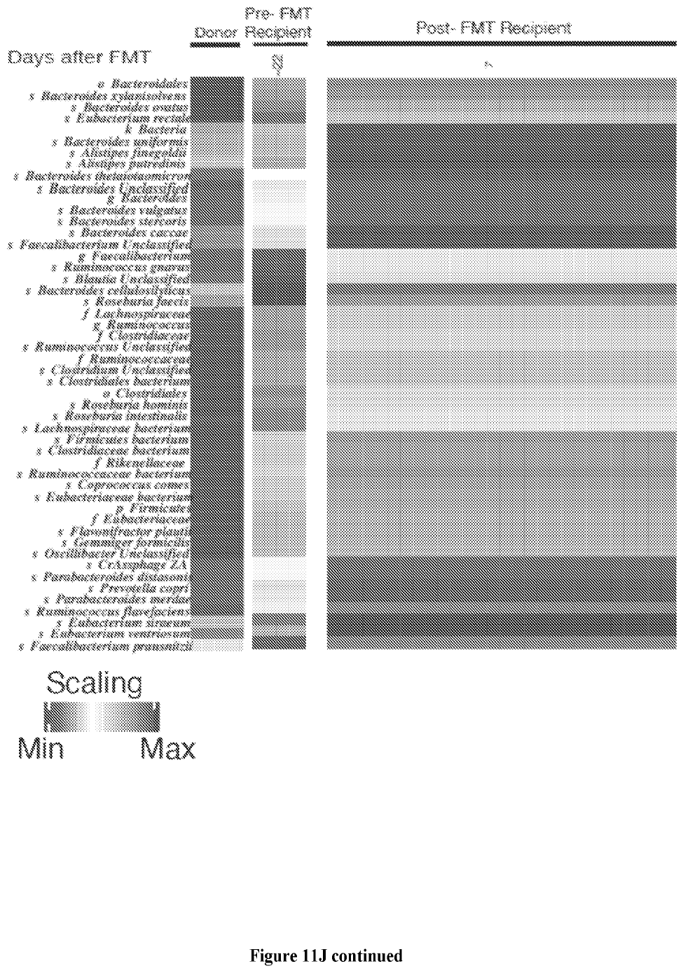

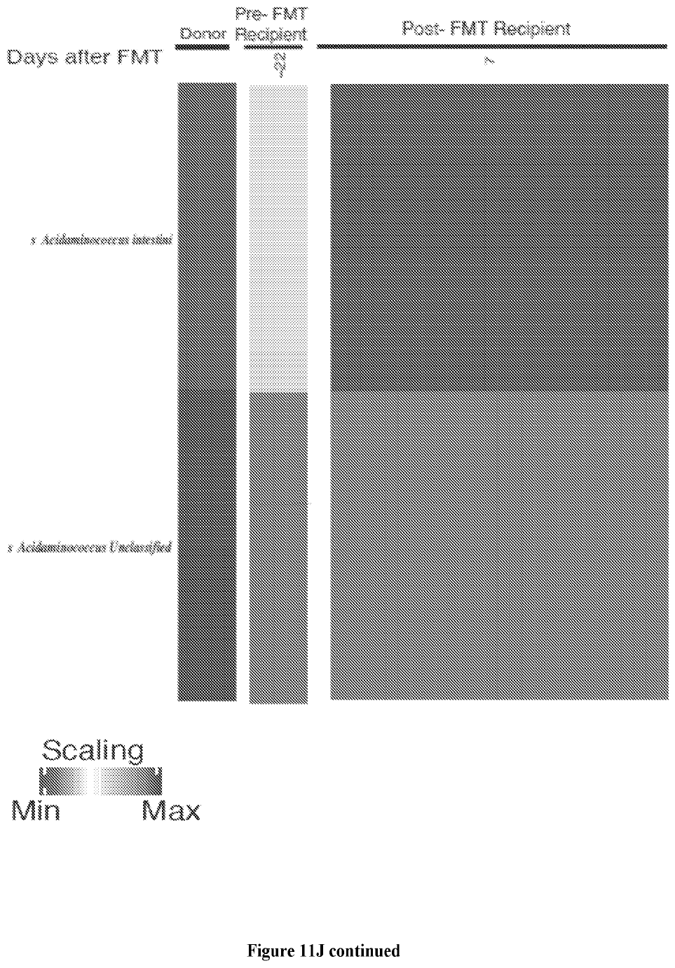

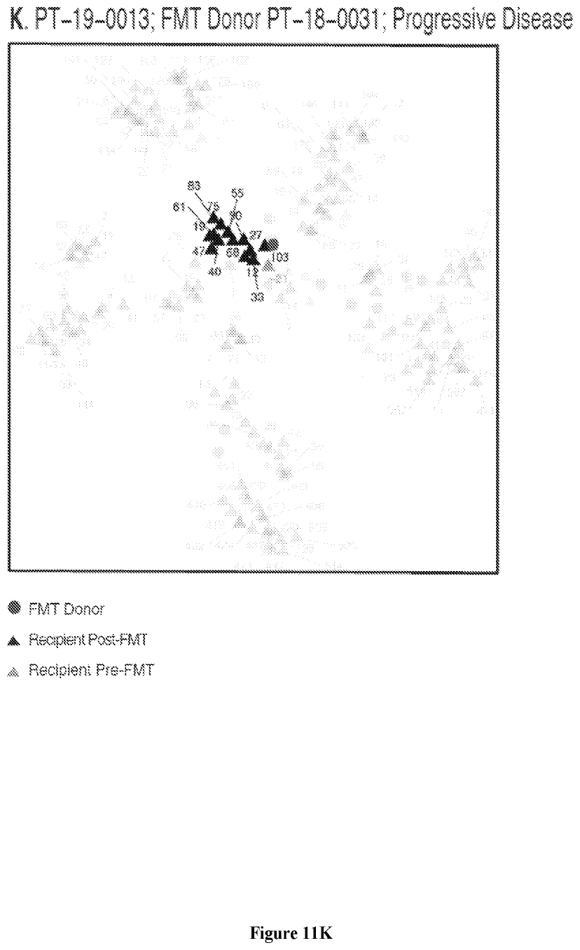

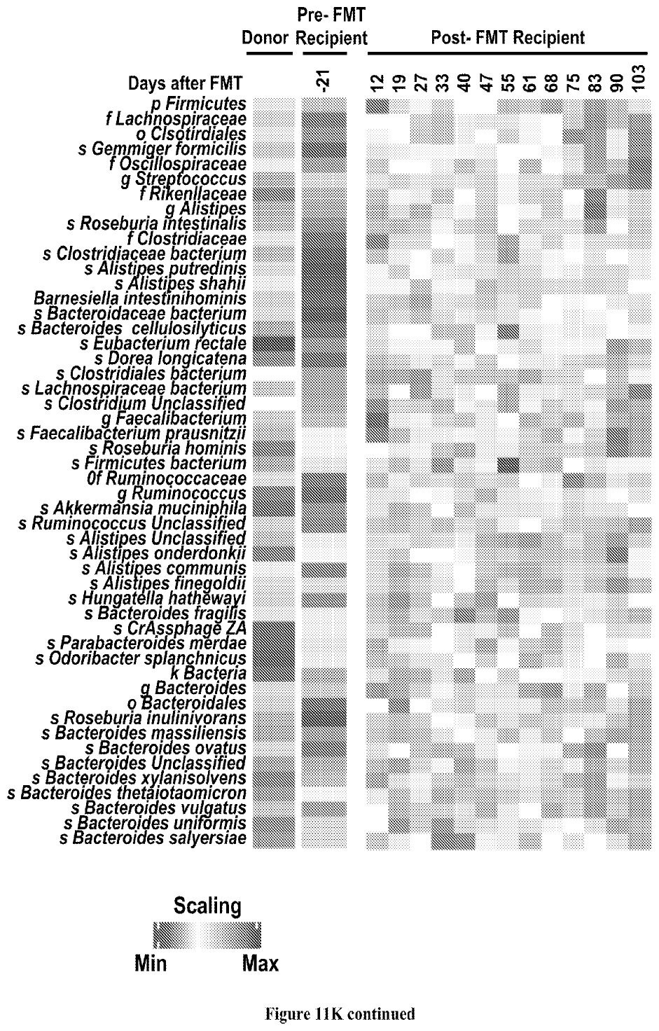

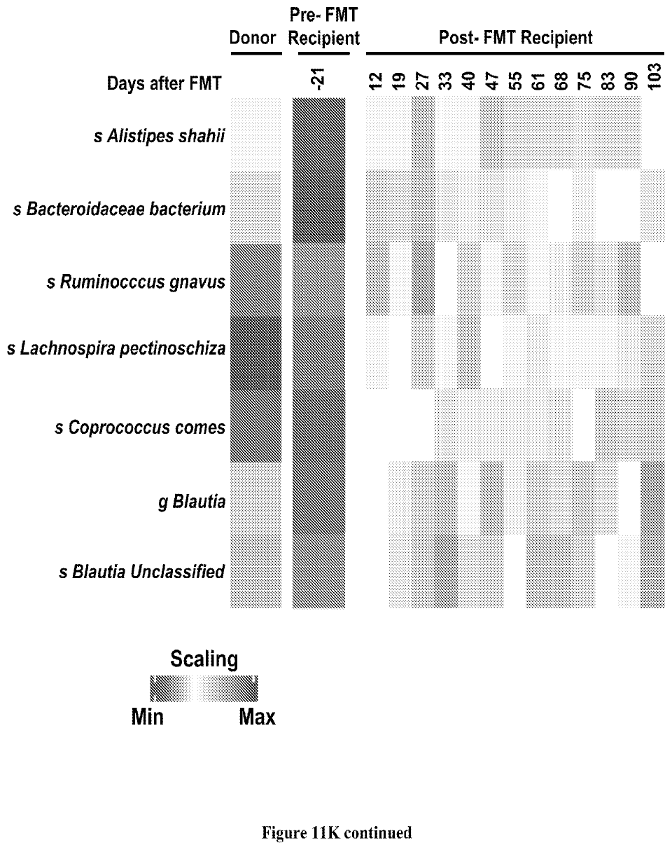

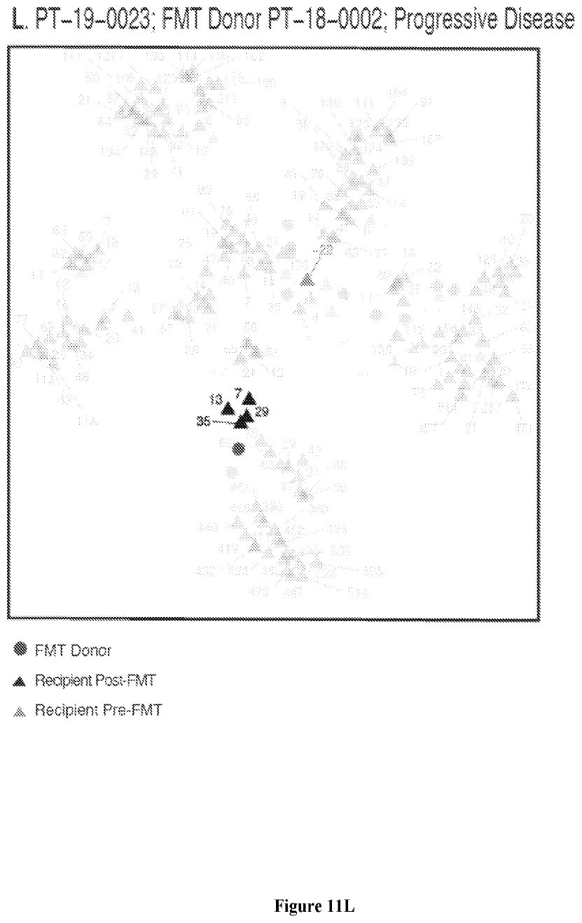

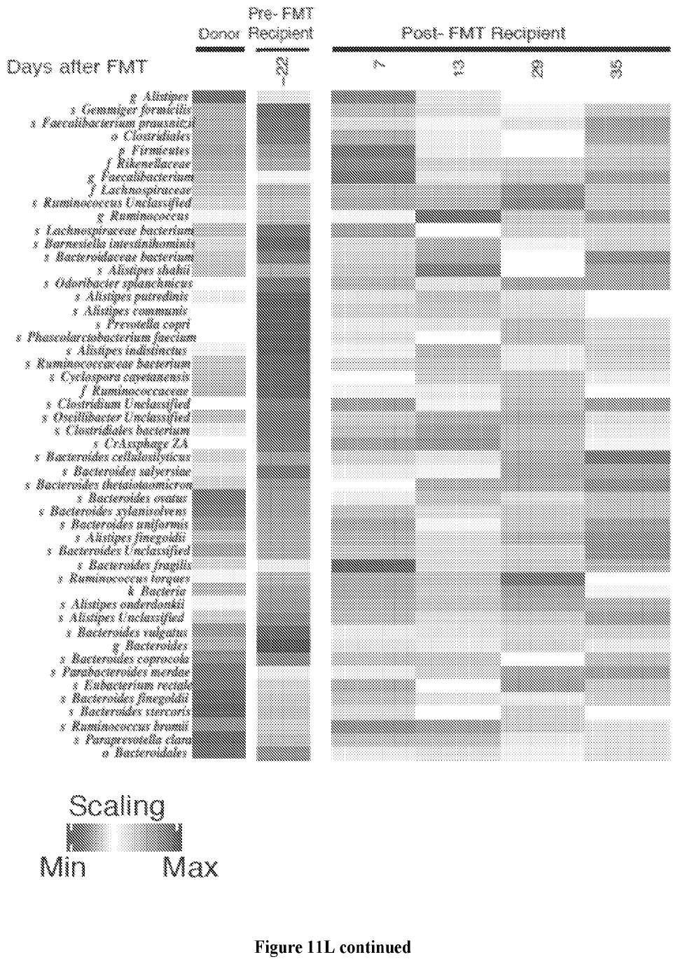

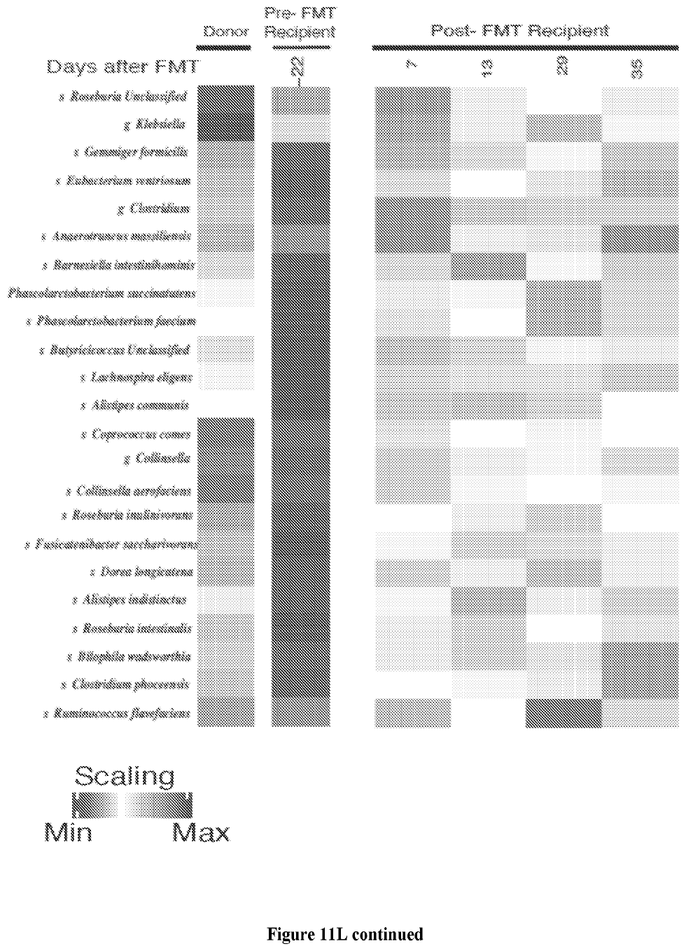

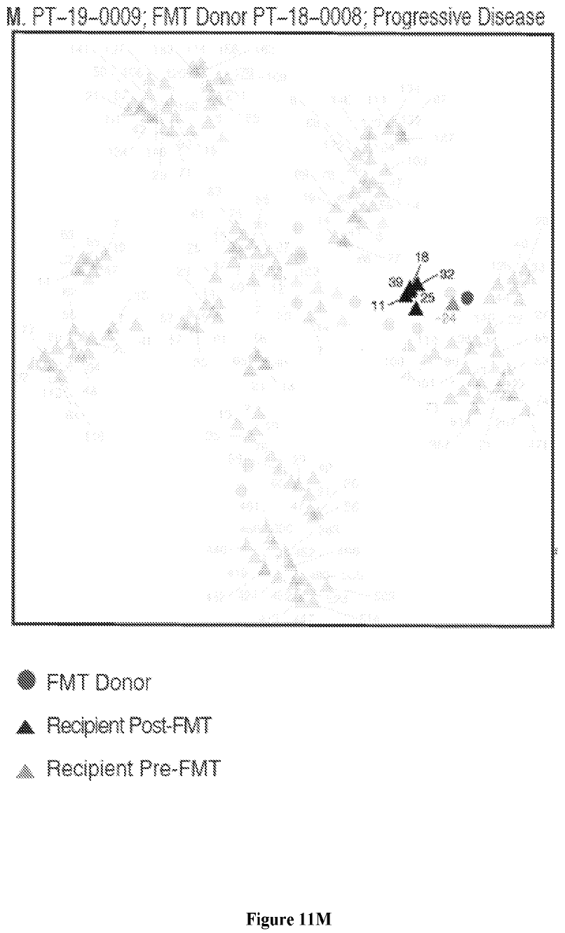

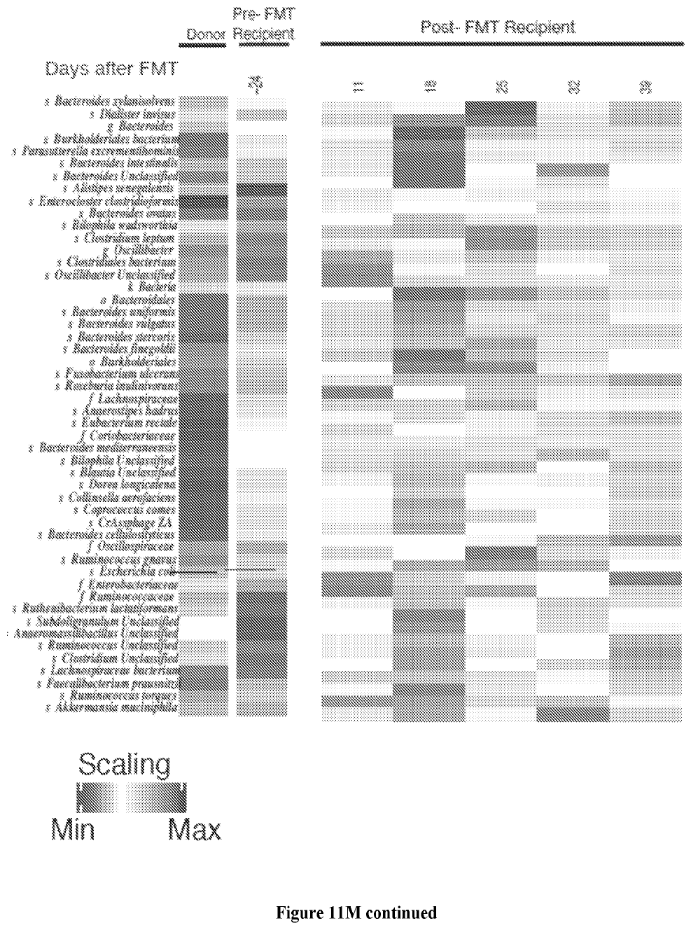

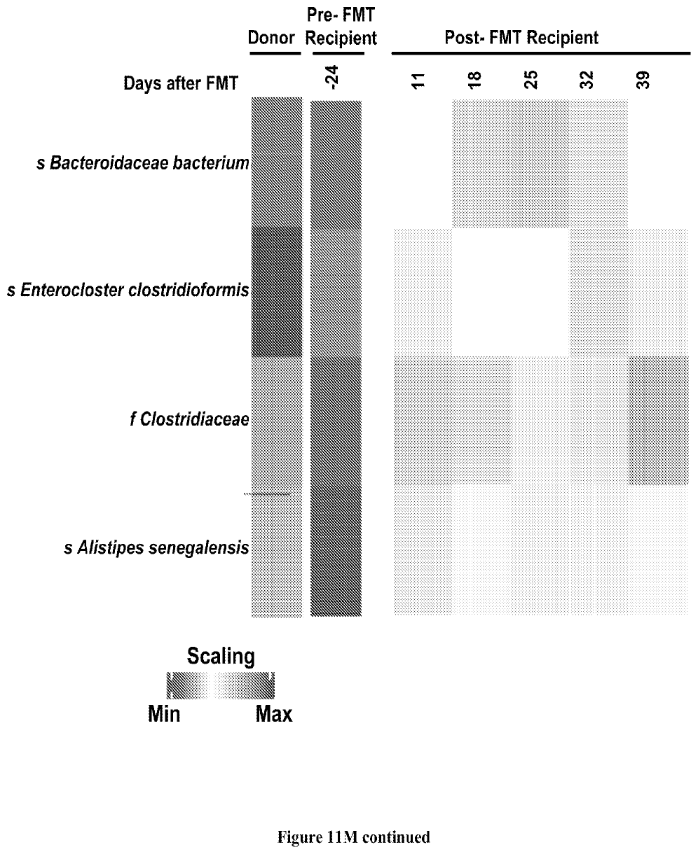

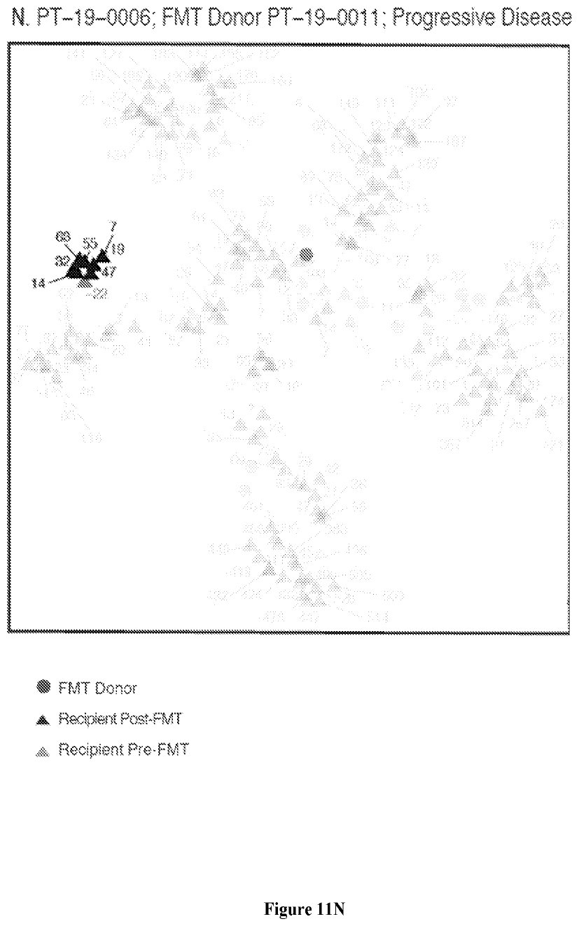

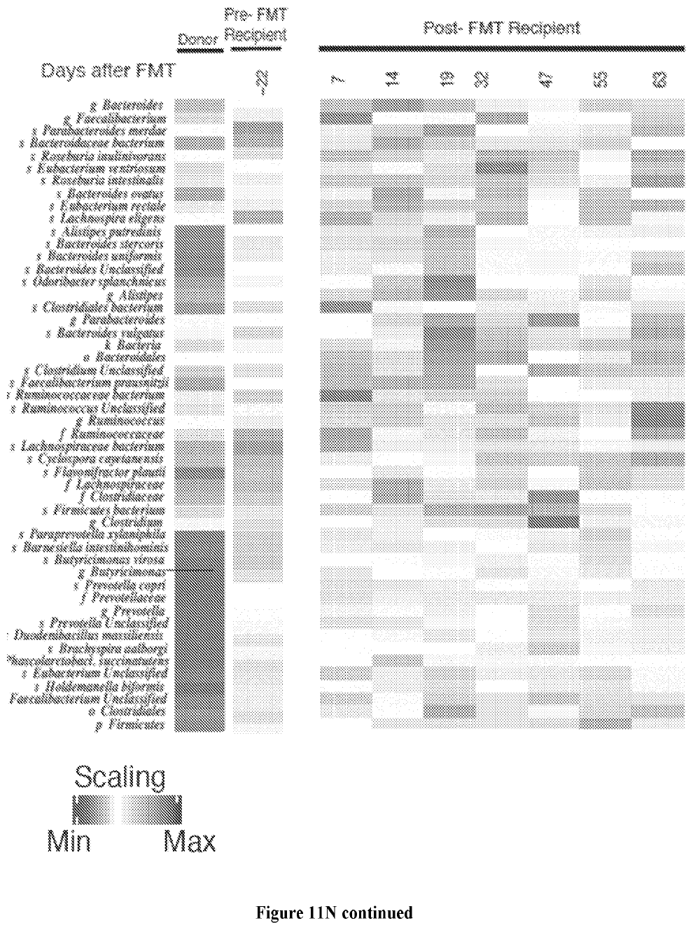

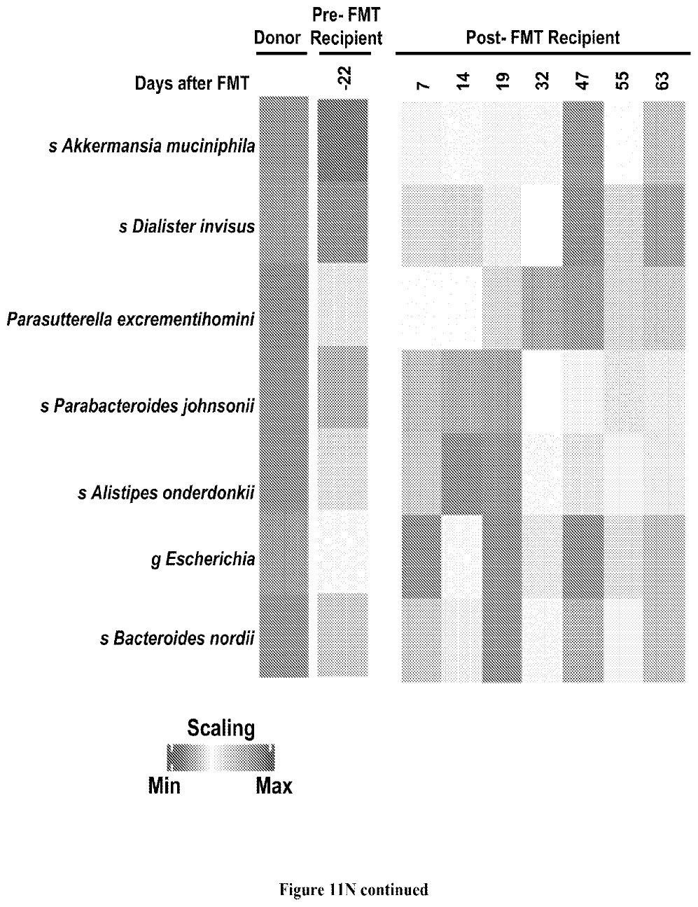

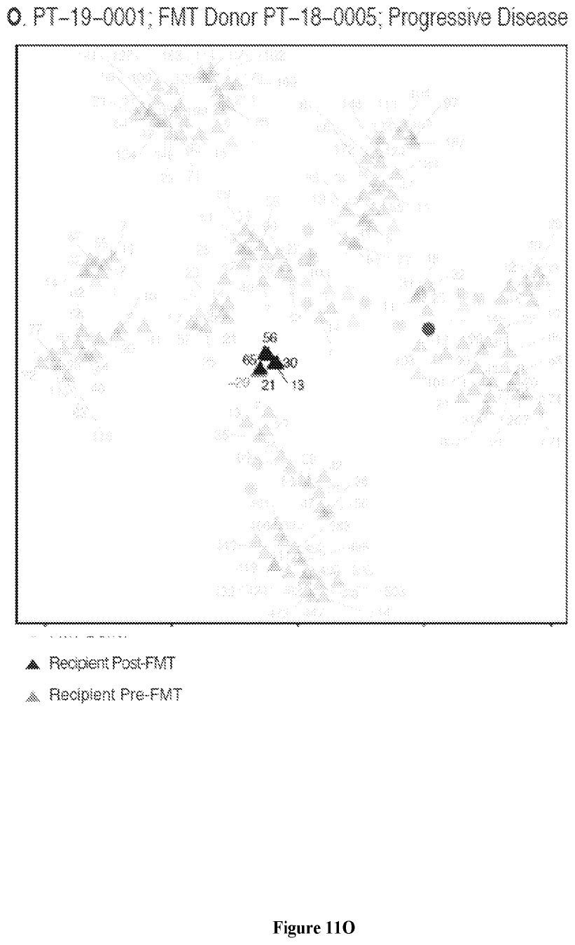

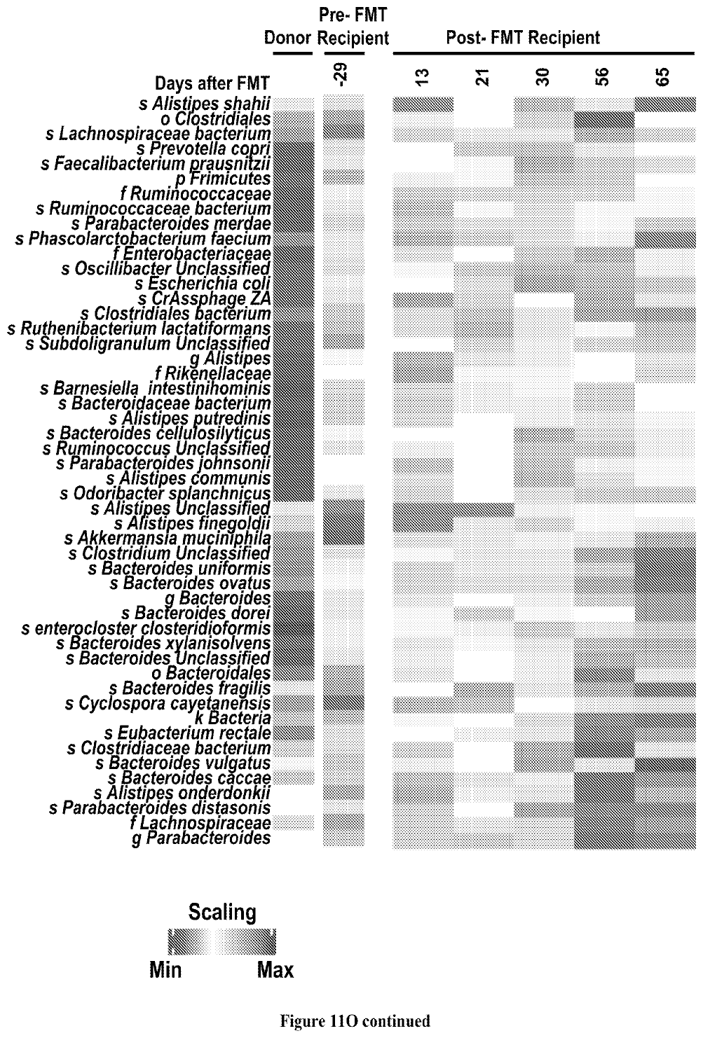

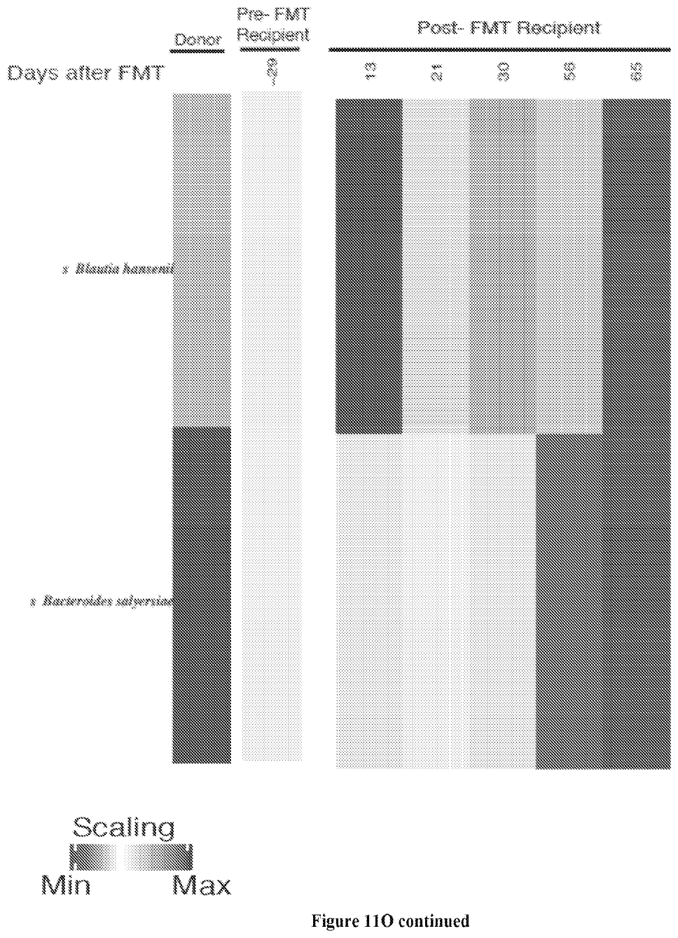

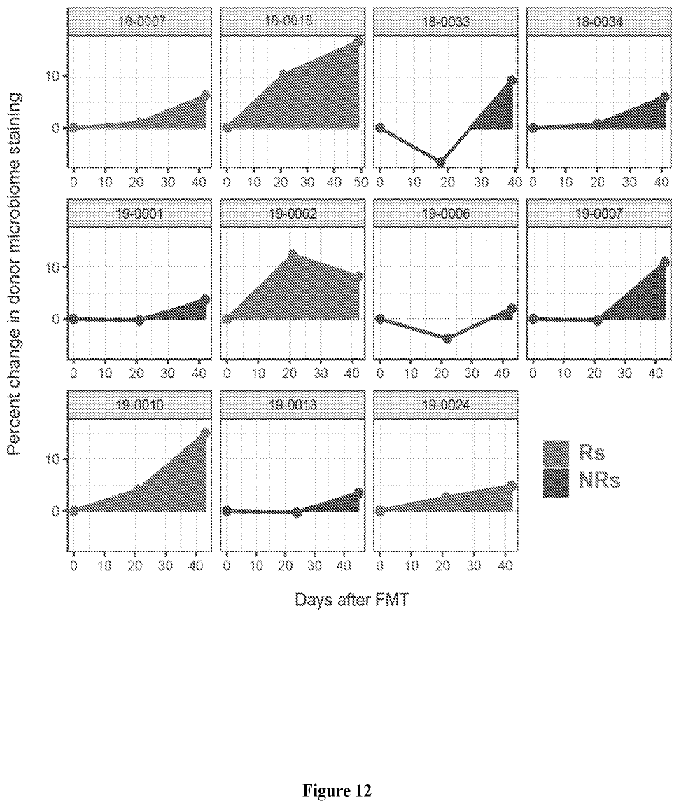

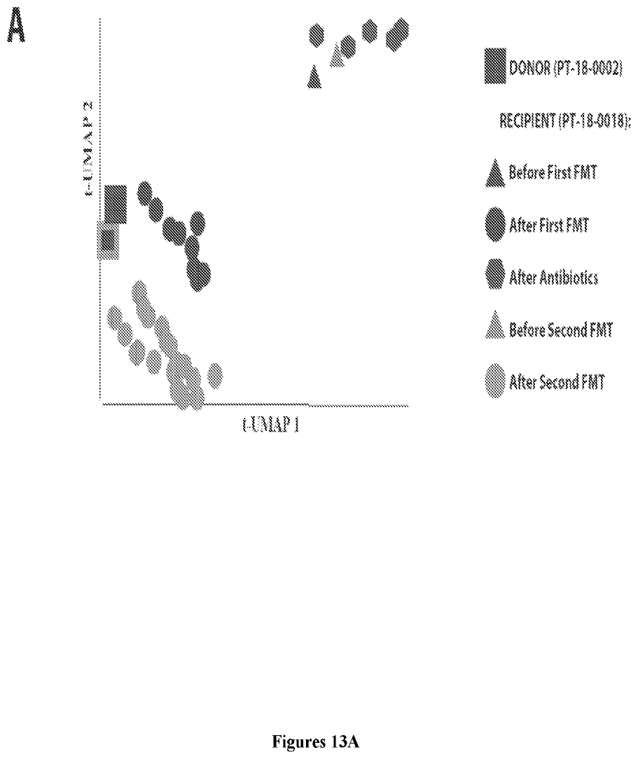

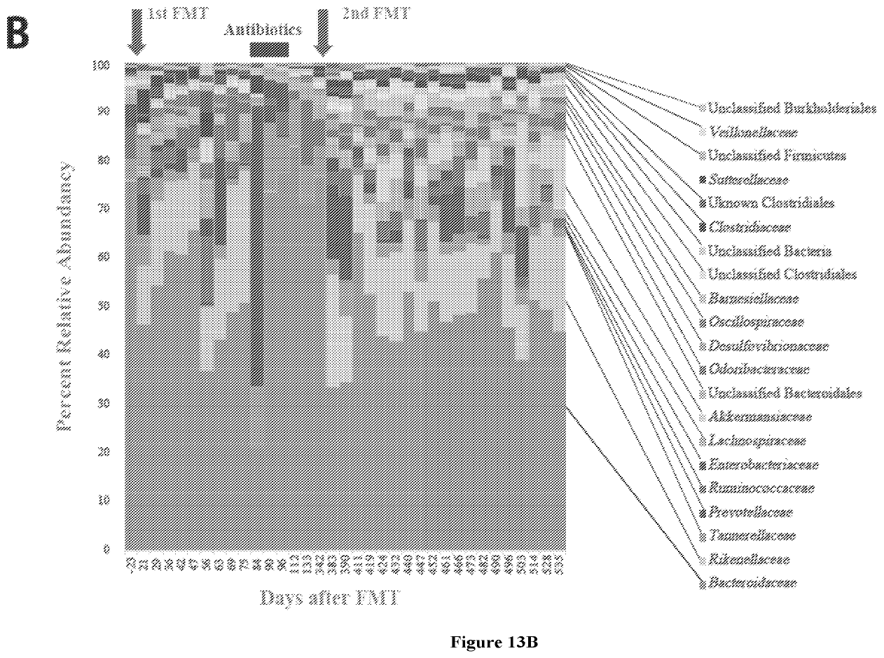

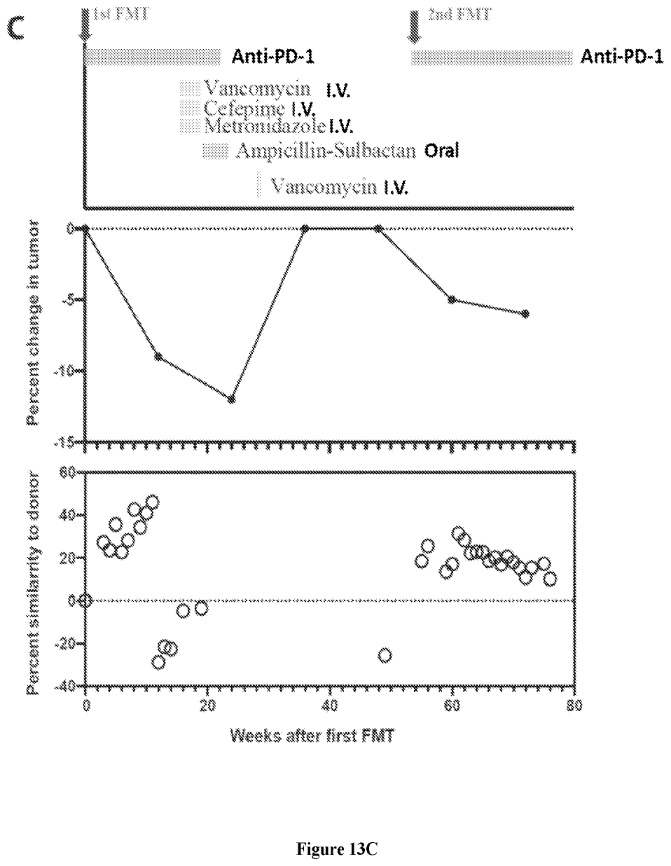

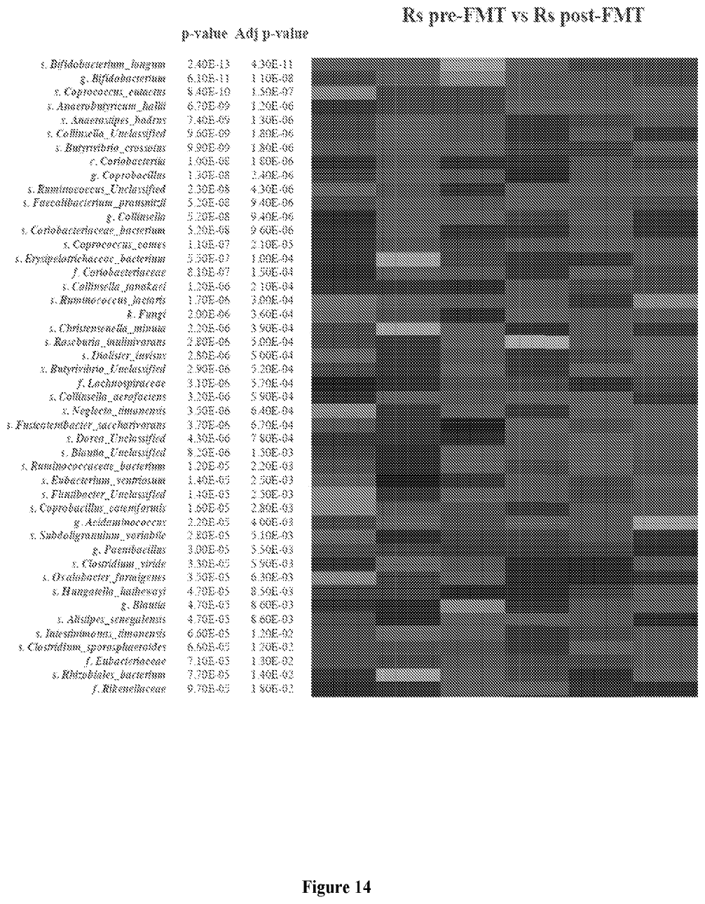

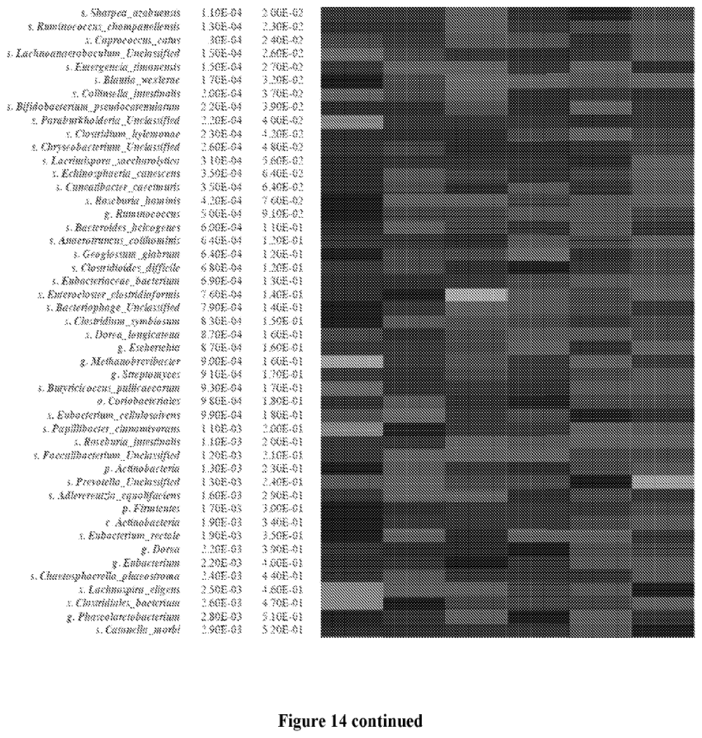

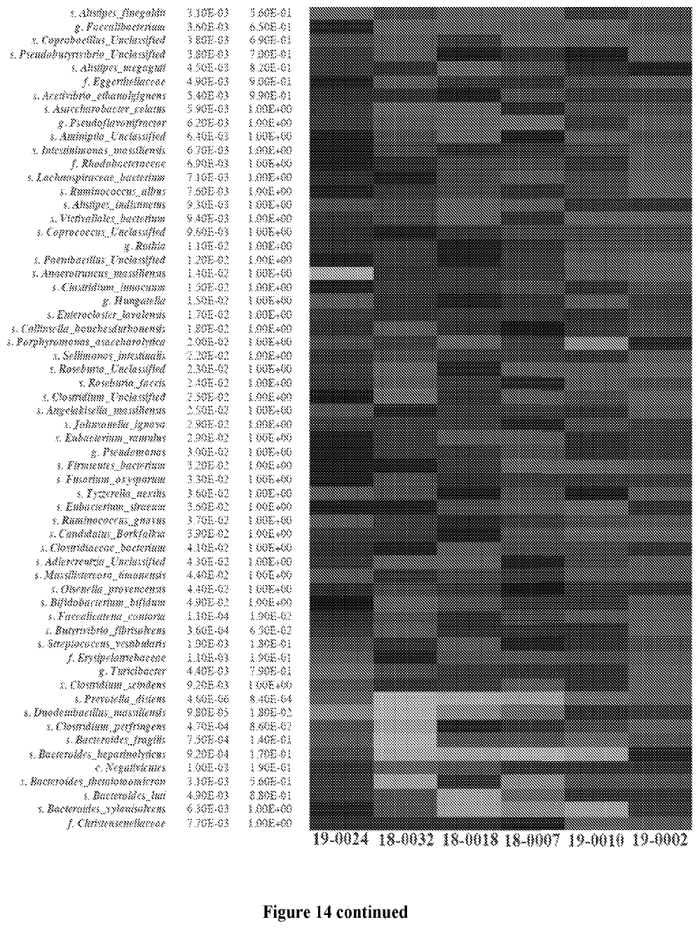

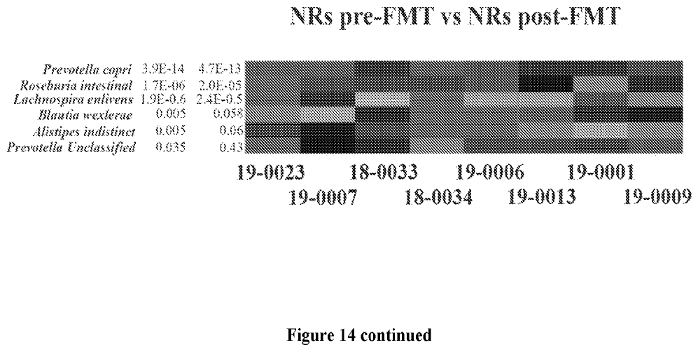

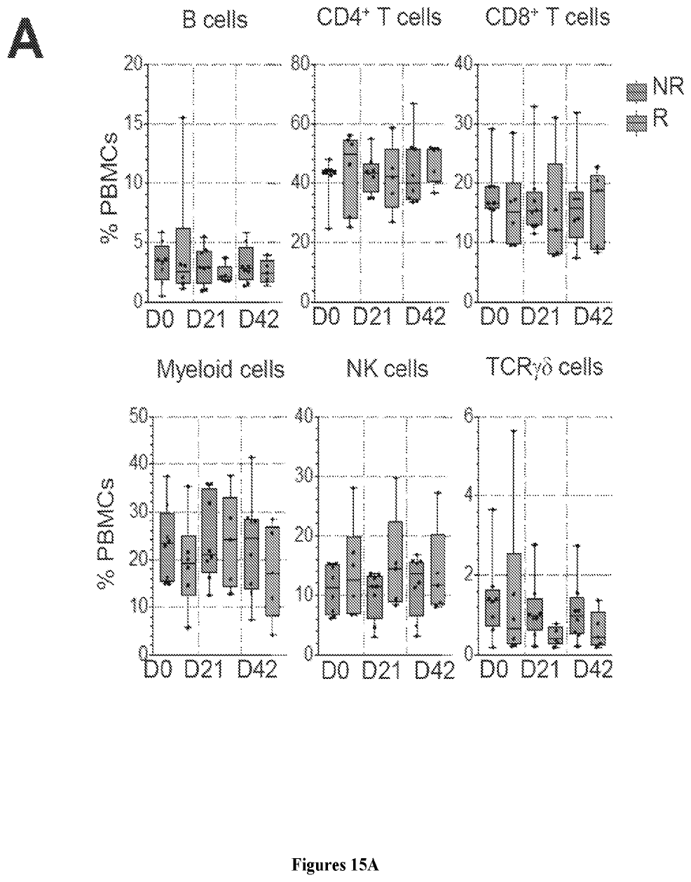

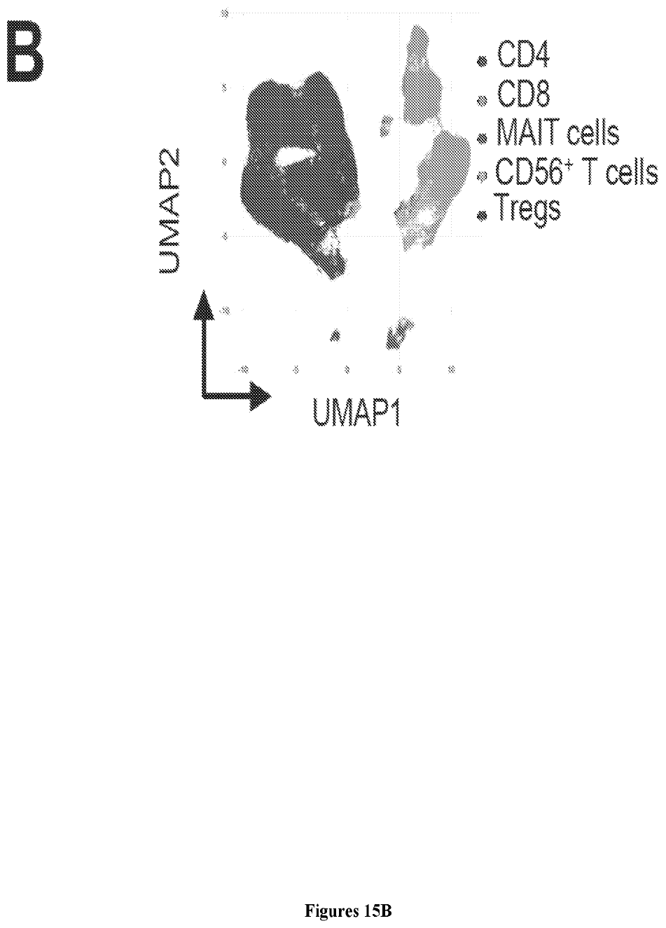

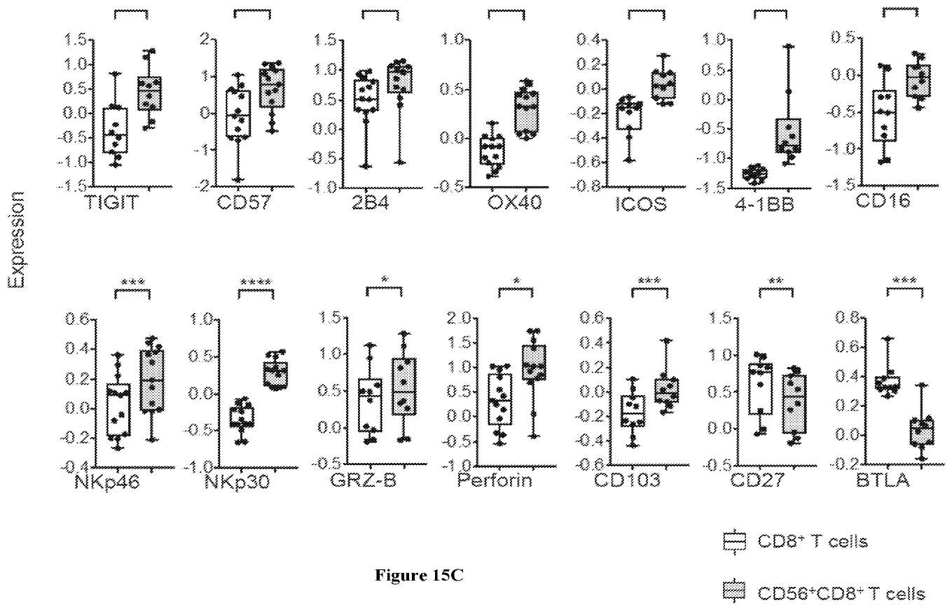

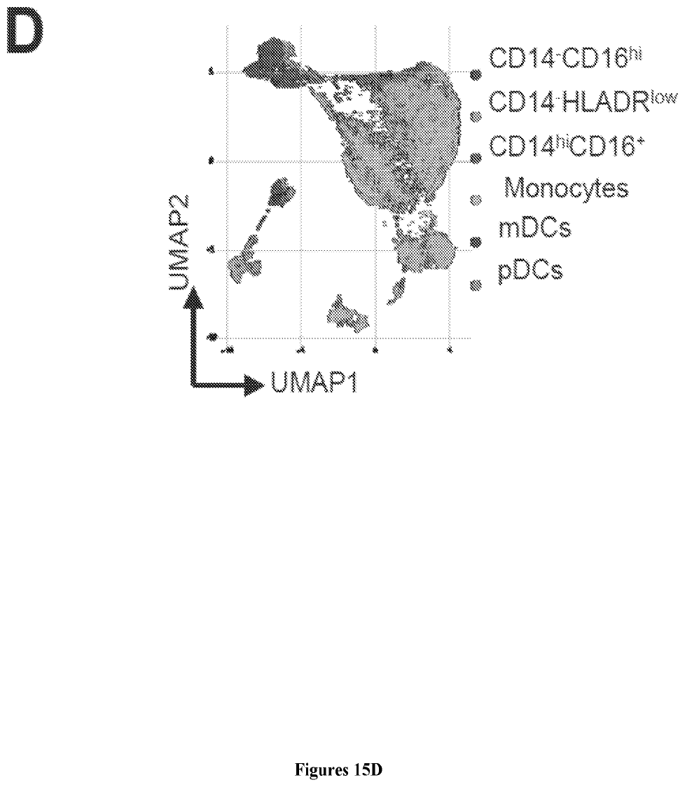

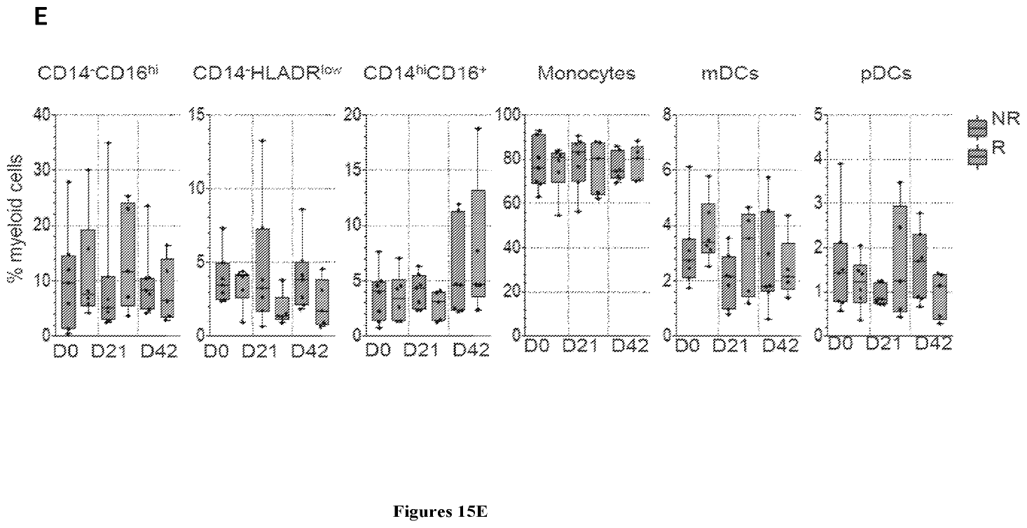

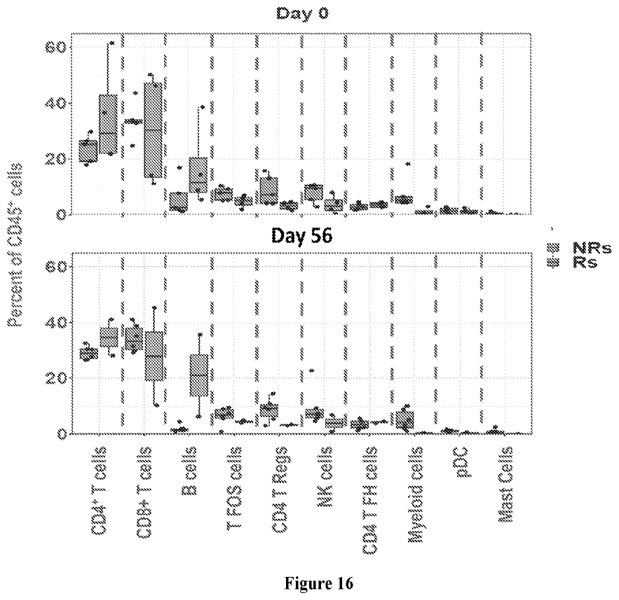

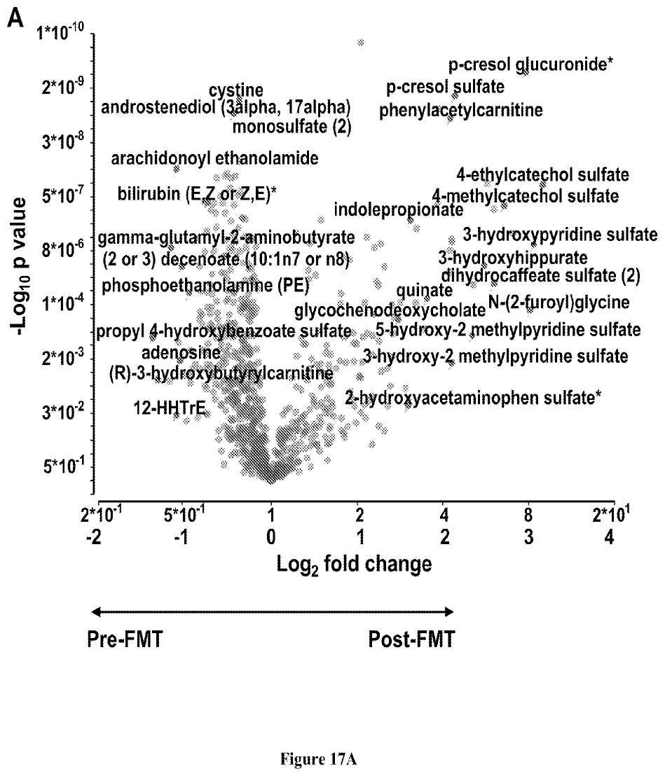

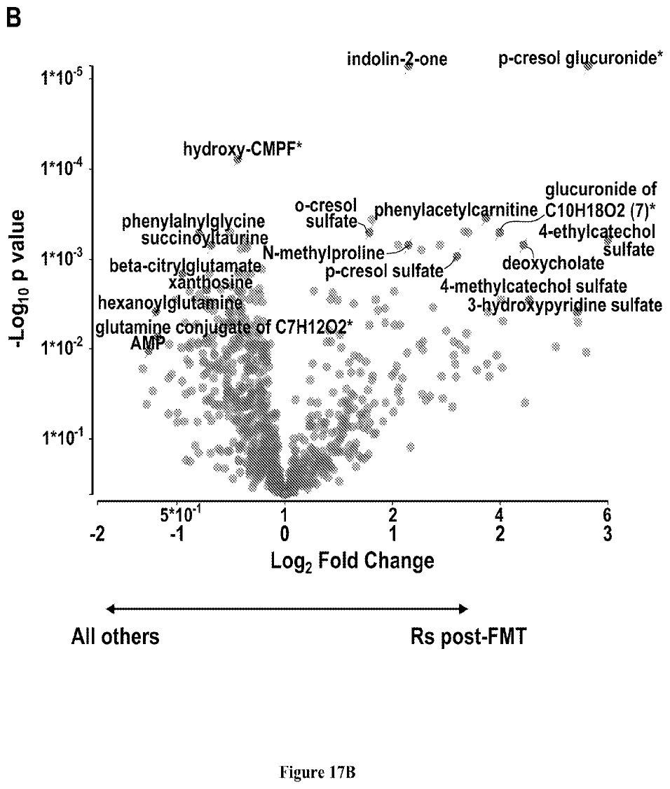

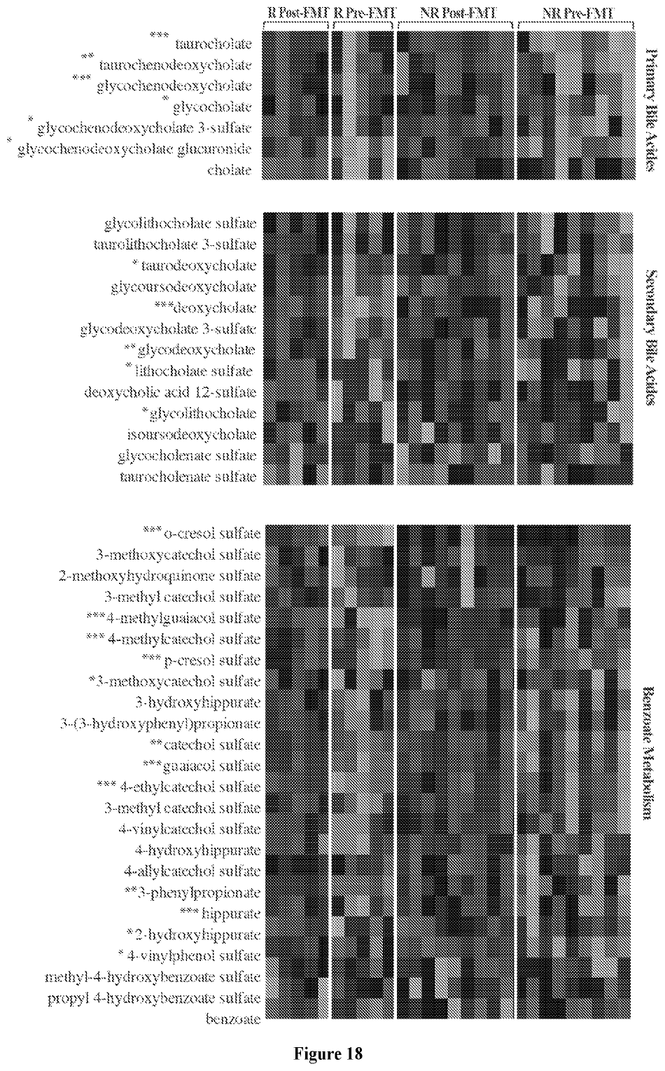

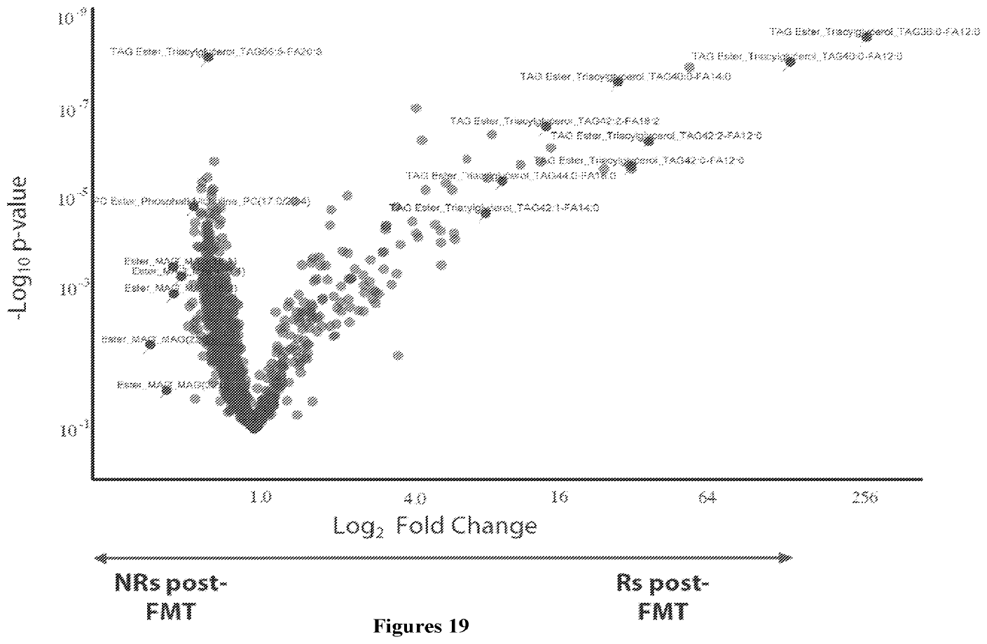

Pre-FMT stool samples are depicted as squares and post-FMT stool samples as triangles. Ellipses encapsulate each recipient's pre- and post-FMT samples, and size of the ellipse spans two standard deviations (SDs) from the centroid. Rs and NRs are distinguished by solid and dotted lines, respectively. PT-19-0026 (PD) is not depicted due to a single post-FMT sample. FIG. 2B shows intra-patient variance of stool samples from donors and recipients following standardization and dimensionality reduction. Donors (n=3) and recipients (n=15) who contributed ≥3 fecal samples are depicted. Data were standardized, PCA was performed, PC loading was computed, and variances of patients for every PC loading were calculated as the SD2/mean and multiplied by the PC variance contribution. Resultant values were added together to produce a combined variance number, which was compared between donors (n=3) and recipients (n=15) using the non-parametric t-test. FIG. 2C shows rate of taxonomic change of stool samples sequentially obtained from treated patients. The rate of taxonomic change for each sample sequentially obtained from each patient was calculated using speed of traversion (Euclidean distances traversed per day) calculated by dividing total Euclidean distance travelled by days. Euclidean distance was calculated using log-transformed normalized taxonomic data from shotgun sequencing between Rs (n=6) and NRs (n=9) using the non-parametric t-test. FIG. 2D shows plot of Euclidean distance over time from patients' gut microbiota to corresponding FMT donor's microbiota. To assess the efficiency of FMT engraftment, Euclidean fitted curves were generated using points on the graph in both NRs (red, above) and Rs (blue, below). Positive percentage of each curve indicates similarity to the corresponding donor while negative percentage indicates changes in the recipient microbiota even further from the donor microbiota. Graphs were normalized by truncating at 60 days in both Rs and NRs. The chi-squared test was calculated using an even distribution as null hypothesis. FIG. 2E shows recipient IgG response against donor microbiota induced by FMT. Donor fecal bacteria were incubated with recipient sera at 1:200 dilutions, washed and stained with PE-labelled anti-human IgG, fixed and analyzed on a flow cytometer. Change in IgG positivity of donor fecal bacteria for each FMT recipient was calculated in relation to the first FMT recipient serum sample available compared to the subsequent two time points up to 50 days later. Percent IgG-positive donor fecal bacteria were assessed and area-under-the-curve (AUC) for percent IgG-positive donor bacteria was calculated adjusting all recipient time points relative to the baseline time point set to zero. Difference in reactivity between sera from Rs and NRs was evaluated by Student's T-test. FIG. 2F shows Cladogram visualization of phylogenetic distribution of differentially abundant taxa pre- and post-FMT in responding patients. Fisher's method for meta-analyses was used to validate statistical significance and calculate effect size of the differential abundances of taxa in Rs (FIG. 14). Differentially abundant taxa are color coded based on relative abundance in post-compared to pre-transplant samples (green—higher; yellow—unchanged; red—lower). Most significantly associated taxa are highlighted at the family level.

FIGS. 3A-3I show single-cell analyses of circulating and tumor-infiltrating immune cells. FIG. 3A shows unsupervised multiparameter flow cytometry analysis of circulating immune cells. UMAP visualization of 100,000 live single cells from Rs (right) and NRs (left) at three time points—pre-treatment (DO), day 21 (D21) and D42 (separated by dashed lines)—from 30-parameter flow panel analysis (n=14) after merging clusters based on expression of CD3, CD4, CD8, CD19, CD14, CD56, Tgd1, and Tgd2. Myeloid cells were identified as lineage-negative cell clusters based on presence or absence of CD14⁺ cells. FIG. 3B shows frequency of CD56⁺CD8⁺ clustered T cells in PBMCs of patients. Whisker boxes show frequencies of CD56⁺CD8⁺ clustered T cells in PBMCs between Rs (right) and NRs (left) at D0, D21, and D42 (separated by dashed lines). We observed a significant increase of CD56⁺CD8⁺ T cells in Rs at D42 using the unpaired t-test (*p<0.05). FIGS. 3C and 3D show phenotypic analysis of circulating CD8⁺ T cells. Whisker boxes show markers that are significantly differentially expressed (normalized mean fluorescence intensity) in CD8⁺ T cells (C) and CCR7⁺CD45RA⁺ naïve, terminally differentiated effector memory CCR7⁻CD45RA⁺ (TEMRA), CCR7⁺CD45RA⁻ effector memory (EM), and CCR7⁻CD45RA⁻ central memory (CM) cells (D) between Rs (n=5-6) (right) and NRs (n=5-7) (right) at the three time points (day 1 (D1), D21, and D42, separated by vertical dashed lines). Analysis was performed on live single CD3⁺ and TCRgd⁻ T cells. In Rs, upregulation of TIGIT, Lag-3, and T-bet post-treatment and downregulation of CD27 in CD8⁺ T cells was observed using the unpaired t-test (*p<0.05). FIG. 3E shows phenotypic analysis of circulating MAIT cells. Whisker-boxes comparing MAIT cells between Rs (n=5-6) (right) and NRs (n=5-7) (left) at the three time points (day 1 (D1), D21, and D42, separated by vertical dashed lines). Analysis was performed on live single CD3⁺ and TCRgd⁻ T cells. In Rs, MAIT cells upregulated granzyme B expression and downregulated CD27 post-treatment using the unpaired t-test (*p<0.05). FIG. 3F shows scRNA-seq analysis of tumor-infiltrating immune cells. UMAP projection of 64,000 CD45³⁰ cells that were clustered and manually identified based upon their expression profile. FIG. 3G shows abundance of myeloid cells and CD4⁺ T regs in CD45⁺ tumor-infiltrating cells. Whisker boxes showing the abundance of myeloid cells and CD4⁺ T regs in CD45⁺ tumor-infiltrating cells on day 0 (DO) and D56. Decreased abundance of myeloid cells and CD4⁺ T regs was observed in Rs compared to NRs using the unpaired t-test (*p<0.05). FIG. 3H shows cell-associated expression of two markers (CXCL8, SPP1) in UMAP projection. These markers are predominantly expressed in suppressive myeloid cells. FIG. 3I shows volcano plots showing the differences in phenotype of CD8⁺ T cells and myeloid cells between Rs and NRs post-FMT. Rs show a CD8⁺ T phenotype with increased activation markers (GZMK, class II HLA genes, CD74), while NRs show a myeloid phenotype with an increased suppressive signature (CXCL8, SPP1) at day 56 post-treatment. Adjusted p-values were obtained by Wilcoxon rank-sum test.

FIGS. 4A-4E show serum proteomics, metabolomics, and lipidomics signatures pre- and post-FMT. FIG. 4A shows PCA and heatmap of serum cytokines of Rs and NRs before and after FMT. Data show that Rs post-treatment (orange) separate from Rs pre-treatment (green), along with NRs pre- (red) and post- (blue) treatment as assessed by two-way analysis of variance (ANOVA) (p<0.05). FIG. 4B shows PCA and heatmap of serum metabolites of Rs and NRs before and after FMT. Data show that Rs post-treatment (orange) separate from Rs pre-treatment (green), along with NRs pre- (red) and post-treatment (blue) as assessed using two-way ANOVA (q<0.05). FIG. 4C shows PCA and heatmap of serum lipidomics of Rs and NRs before and after FMT. Serum lipidomic analyses show that Rs post-treatment (orange) distinctly clustered separately from Rs pre-treatment (green), along with NRs pre- (red) and post- (blue) treatment, as assessed using two-way ANOVA (q<0.05). FIG. 4D shows Transkingdom network analysis of multi-omic data. Data for microbial (octagons), metabolites (squares), cytokines (triangles), and multi-parameter flow cytometry (hearts) were analyzed to identify highly differentially abundant elements between Rs and NRs to FMT/pembrolizumab. To identify nodes (i.e., any of these four types of elements) and their groups with potential contribution to a regulatory activity, a “transkingdom” network integrating omics data was constructed using their correlations within the different groups (Rs or NRs, pre- or post-FMT/pembrolizumab). Network interrogation revealed that “microbiome” and “metabolite” as well as “microbiome” and “cytokine” subnetworks were the most inter-connected. We identified a dense subnetwork (module) containing the highest number of nodes from different omics data (nodes highlighted in yellow, positively correlated edges in red and negative correlatively edges in blue). FIG. 4E shows subnetwork identified in FIG. 4D. Network analyses established that CXCL8/IL-8, IL-10, and CCL3/MIP-1α were positively correlated with organisms enriched in NRs pre-treatment (B. uniformis, B. nordii, P. faecium, etc.) and negatively correlated with organisms enriched in Rs post-treatment (e.g., R. flavefaciens, F. prausnitzii).

FIG. 5 shows clinical trial schema of a Phase II study of anti-PD-1 responder-derived FMT and pembrolizumab in anti-PD-1-refractory melanoma. Eligible subjects were melanoma patients primary refractory to anti-PD-1 therapy. Patients had received 2:2 cycles of anti-PD(L)1 therapy over 2:6 weeks with best response of PD or SD for <6 months as assessed by RECIST v1.1. Disease progression was confirmed on at least one imaging study before enrollment by an independent radiologist. Other eligibility criteria were no CNS disease, biopsy-amenable disease, and stable organ function. Following screening, which included extensive serological testing (stool, blood) for potentially transmissible infectious agents, and tumor biopsy, patients received a FMT derived from individual melanoma patients with long lasting objective response to anti-PD1 therapy. FMT administered colonoscopically on day 0 along with pembrolizumab (200 mg). Pembrolizumab was repeated every 3 weeks. Tumors were rebiopsied at weeks 9-12, and blood and stool were resampled periodically. Restaging scans were performed at weeks 9-12 and repeated every 9-12 weeks while on study. Patients remained on study until intolerable toxicity, RECIST v1.1 confirmed disease progression, or completion of 35 cycles of pembrolizumab.

FIG. 6 shows testing methodology used to evaluate recipient and donor suitability.

FIG. 7 shows representative CT scan images from patient PT-18-0007 treated on Phase II study of anti-PD-1 responder-derived FMT and pembrolizumab in anti-PD-1-refractory melanoma.

FIG. 8 shows representative CT scan images from patient PT-18-0032 treated on Phase II study of anti-PD-1 responder-derived FMT and pembrolizumab in anti-PD-1-refractory melanoma.

FIGS. 9A-9D show metagenomic analyses of fecal microbiota from FMT donors (n=7) compared to pre-FMT recipients. FIG. 9A shows dimensionality reduction using t-distributed uniform manifold approximation and projection (t-UMAP) plot of microbial taxa abundances by LKT of FMT donors at different time points. Each color corresponds to a different FMT donor. FIG. 9B shows FMT donor recipient combinations. The anti-PD-1 response status of the donors (CR or PR) and the clinical response in the recipients are reported. The status of the FMT donor (CR or PR) did not significantly affect the probability of clinical response in the recipients [x2 (n=15)=0.4167; p=0.52]. FIG. 9C shows Alpha-diversity (Inverse Simpson Index) of the fecal microbiota of FMT donors (CR or PR) and recipients before FMT (Rs and NRs). FIG. 9D shows taxonomic composition distribution histograms at family level for different fecal microbiota samples obtained from the 7 donors and one sample each (n=15) of recipients pre-FMT.

FIG. 10 shows intra-patient variability of stool samples from donors and recipients following standardization and dimensionality reduction. Donors (n=3) and recipients (Rs=6 and NRs=9) who contributed 2:3 fecal samples are depicted. Data were standardized, PCA was performed, PC loading was computed, and variances of patients for every PC loading were calculated as the SD2/mean and multiplied by the PC variance contribution. Resultant values were added together to produce a combined variance number, which was compared between donors and recipients using the non-parametric t-test.

FIGS. 11A-11O show visualization of metagenomic data of gut microbiome from patients. each recipient patient data are depicted in a separate panel, starting with Rs (PT-18-0032, PT-18-0007, PT-19-0024, PT-18-0018, PT-19-0002, PT-19-0010) and followed by NRs (PT-18-0033, PT-18-0034, PT-19-0001, PT-19-0006, PT-19-0007, PT-19-0009, PT-19-0013, PT-19-0023, PT-19-0026). Results indicate that FMT/pembrolizumab demonstrated substantial colonization by donor microbiota, predominantly in Rs. For each recipient patient (FIGS. 11A-11O): Left: t-UMAP visualization of relative abundances of LKTs of samples involved in all FMT events depicting all samples (light grey triangles), recipient-specific samples (black triangles), pre-treatment sample (green triangle), and donor sample (red circle). Numbers denote the number of days since FMT. Each recipient has only one green triangle, with the exception of PT-18-0018 who received two FMT infusions (two green triangles indicate the two last samples before each FMT treatment); center: relative abundance heatmap of the top 50 most variant LKT across samples. Numbers over each column denote the number of days since FMT. LKTs whose relative abundance was <50 PPM and taxon genome completeness was <5% in at least 5% of samples were discarded; right: engraftment of the LKT present in the donor infusate but not detectable in pre-FMT recipient samples. Relative abundance heatmap of LKTs that were <100 PPM in the pre-FMT recipient sample and >100 PPM in the donor sample. Scaled relative abundances of these LKTs are shown across the samples. Numbers over each column denote the number of days since the FMT.

FIG. 12 shows recipient serum IgG response against donor microbiota following FMT. Donor fecal bacteria were incubated with recipient sera at 1:200 dilutions, washed and stained with PE-labelled anti-human IgG, fixed and analyzed on a flow cytometer. Change in IgG positivity of donor fecal bacteria for each FMT recipient was calculated in relation to the first FMT recipient serum sample available compared to the subsequent two time points up to 50 days later. The shaded area in each box indicates area under the curve (AUC).

FIGS. 13A-13C show visualization of metagenomic data of gut microbiome from patient PT-18-0018. Patient PT-18-0018 eleven weeks after FMT developed a soft tissue infection requiring intravenous and subsequent oral antibiotics. A second transplant from the same donor was performed nearly 1 year after initial FMT. FIG. 13A shows t-UMAP visualization of fecal microbiota composition of sequential samples from patient PT-18-0018 and, depicted with a square, the two infusates from donor PT-18-0002 used for the first (square with dark grey border) and second (square with light grey border) FMT. FIG. 13B shows taxonomic composition distribution histograms at family level for the sequential fecal microbiota samples obtained from patient PT-18-0018. FIG. 13C shows treatment schedule, tumor growth and Euclidean distance over time from patients' gut microbiota to the corresponding FMT donor's microbiota. Positive percentage indicates similarity to the corresponding donor while negative percentage indicates changes in the recipient microbiota even further from the donor microbiota.

FIG. 14 shows heatmap of differentially abundant taxa between pre- and post-FMT/pembrolizumab samples from Rs. Changes of microbiome in Rs and NRs patients were individually evaluated and summary statistics for all samples was calculated using Fisher's method for meta-analyses to estimate statistical significance. Taxa with p<0.05 is shown, Both, p-values and Bonferroni-adjusted p-values are shown next to the taxa. The heatmaps show differentially abundant taxa that were first sorted based on sign of its change (positive or negative) and then ranked by significance and ratio values, with highest significant taxa being on top of its corresponding list.

FIGS. 15A-15E show unsupervised spectral flow cytometry analysis and identification of a highly activated CD56⁺CD8⁺ T Cell Subset in the PBMCs of PD-1 Refractory Patients. FIG. 15A shows Whisker boxes showing the frequencies of immune cells in PBMCs between Rs (right) and NRs (left) on day 1 (D1), D21, and D42 (separated by vertical dashed lines) after merging clusters based on expression of CD3, CD4, CDS, CD19, CD14, CD56, Tgd1, and Tgd2. Myeloid cells were identified as lineage-negative cell clusters with or without CD14+ cells. FIG. 15B shows UMAP visualization of 10,000 events from the TCRgd-CD3+ T-cell cluster showing CD8⁺ T cells, CD4⁺ T cells, CD25⁺CD127⁻ Tregs, TCRav7.2⁺CD161⁺ MAIT cells, and CD3⁺CD56⁺ T cells. FIG. 15C shows comparison of expression (normalized MFI) of the indicated makers by CD8⁺ and CD56⁺CD8⁺ T-cell clusters pre-treatment from four different flow panels (n=10-14). P-values were obtained by parametric or non-parametric paired t-tests according to gaussian or non-gaussian distribution of data with: *p<0.05; **p<0.01; ***p<0.001; ****p<0.0001. FIG. 15D shows UMAP visualization of 10,000 events from the myeloid cell subsets showing CD14⁻CD11c⁺HLADR⁺ mDCs, CD123⁺BDCA2⁺HLA-DR⁺pDCs, CD14⁺CD16^{hi} cells, CD14⁻CD16⁺ cells, and CD33^{low}CD14⁻HLADR^{low} myeloid cells. FIG. 15E shows Whisker boxes showing percentages of myeloid cell subsets between Rs (right) and NRs (left) for three time points (day 1 (D1), D21, and D42, separated by vertical dashed lines) in the myeloid population. FIGS. 15D and 15E show that CD3⁺, CD19⁺, and CD33⁻CD56⁺ cells were excluded from the analysis.

FIG. 16 shows abundance of ScRNA-seq clusters in CD45⁺ tumor-infiltrating cells. Whisker boxes showing the abundance of each cluster before and after (day 56) treatment in Rs (right) and NRs (left). Abundance was calculated by dividing the number of cells in a sample by the total number of cells in that sample. *p<0.05.

FIGS. 17A-17B shows relationship between serum metabolites and outcome in response to FMT. Volcano plots of inverse log10 p-values and log2 fold change of serum metabolites for FIG. 17A) all patients at pre- and post-FMT time points and FIG. 17B) Rs post-FMT vs all others. Metabolites increased post-transplant in Rs are displayed on the right side of the plots, and those that decreased are shown on the left. Labels of several example metabolites with most significant fold changes and p-values are shown.

FIG. 18 shows visualization of the Selected Significant Metabolic Families in the Serum of Rs and NRs pre- and post-FMT. All detected primary (upper panel) and secondary (middle panel) bile acids, as well as metabolic compounds from benzoate metabolism (lower panel) regardless of significance are plotted as a heatmap. Two-way ANOVA was used to calculate significance and the adjusted p-value results were denoted for q<0.001 as ***, q<0.01 as ** and q<0.05 as *.

FIG. 19 shows relationship between serum lipids and outcome in response to FMT/Pembrolizumab. Lipidomic dataset analysis. Volcano plot of log2 ratios and p-value of comparison of Rs post-FMT and NRs post-FMT.

### DETAILED DESCRIPTION OF THE INVENTION

Anti-programmed cell death protein 1 (PD-1) therapy provides long-term clinical benefits to patients with cancer (e.g., melanoma). The disclosure herein shows that fecal microbiota transplantation (FMT) and anti-PD-1 antibody can modify the gut microbiome and reprogram the tumor microenvironment to overcome resistance to anti-PD-1 antibody (e.g., in an advanced melanoma patient).

Accordingly, disclosed herein are methods for treating a cancer in a subject comprising administering to the subject a therapeutically effective amount of a fecal sample and an anti-PD-1 antibody, wherein the fecal sample is derived from a donor that is responsive to an anti-PD-1 antibody. In some examples, the subject is less responsive to the anti-PD-1 antibody therapy than the donor or non-responsive to the anti-PD-1 antibody. This method can be applied in combination with one or more administrations of the anti-PD-1 antibody.

### TERMINOLOGY

As used in the specification and claims, the singular form “a,” “an,” and “the” include plural references unless the context clearly dictates otherwise. For example, the term “a cell” includes a plurality of cells, including mixtures thereof.

The term “about” as used herein when referring to a measurable value such as an amount, a percentage, and the like, is meant to encompass variations of ±20%, ±10%, ±5%, or ±1% from the measurable value.

“Administration” of “administering” to a subject includes any route of introducing or delivering to a subject an agent. Administration can be carried out by any suitable route, including oral, topical, intravenous, subcutaneous, transcutaneous, transdermal, intramuscular, intra-joint, parenteral, intra-arteriole, intradermal, intraventricular, intracranial, intraperitoneal, intralesional, intranasal, rectal, vaginal, by inhalation, via an implanted reservoir, or via a transdermal patch, and the like. Administration includes self-administration and the administration by another.

The term “cancer” as used herein is defined as disease characterized by the rapid and uncontrolled growth of aberrant cells. Cancer cells can spread locally or through the bloodstream and lymphatic system to other parts of the body. Examples of various cancers include but are not limited to, breast cancer, prostate cancer, ovarian cancer, cervical cancer, skin cancer, pancreatic cancer, colorectal cancer, renal cancer, liver cancer, brain cancer, lymphoma, leukemia, lung cancer and the like. In some embodiments, the cancer is an anti-PD-1 refractory cancer. In some embodiments, the cancer is a melanoma. In some embodiments, the cancer is an anti-PD-1 refractory melanoma.

As used herein, the term “comprising” is intended to mean that the compositions and methods include the recited elements, but not excluding others. “Consisting essentially of” when used to define compositions and methods, shall mean excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions of this invention. Embodiments defined by each of these transition terms are within the scope of this invention.

A “control” is an alternative subject or sample used in an experiment for comparison purposes. A control can be “positive” or “negative.” In some embodiments, the control described herein is a negative control wherein the control subject or control population receives an immune checkpoint inhibitor such as an anti-PD-1 antibody, but does receive a fecal sample according to the present invention.

“Inhibit”, “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

As used herein, the terms “metastasis” and “metastatic” are meant to refer to the process in which cancer cells originating in one organ or part of the body, with or without transit by a body fluid, and relocate to another part of the body and continue to replicate. Metastasized cells can subsequently form tumors which may further metastasize. Metastasis thus refers to the spread of cancer, from the part of the body where it originally occurred, to other parts of the body.

The terms “metastatic cells”, “metastatic tumor cells”, and “advanced tumor cells” are used interchangeably.

“Pharmaceutically acceptable” component can refer to a component that is not biologically or otherwise undesirable, i.e., the component may be incorporated into a pharmaceutical formulation of the invention and administered to a subject as described herein without causing significant undesirable biological effects or interacting in a deleterious manner with any of the other components of the formulation in which it is contained. When used in reference to administration to a human, the term generally implies the component has met the required standards of toxicological and manufacturing testing or that it is included on the Inactive Ingredient Guide prepared by the U.S. Food and Drug Administration.

“Pharmaceutically acceptable carrier” (sometimes referred to as a “carrier”) means a carrier or excipient that is useful in preparing a pharmaceutical or therapeutic composition that is generally safe and non-toxic, and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents.

As used herein, the term “carrier” encompasses any excipient, diluent, filler, salt, buffer, stabilizer, solubilizer, lipid, stabilizer, or other material well known in the art for use in pharmaceutical formulations. The choice of a carrier for use in a composition will depend upon the intended route of administration for the composition. The preparation of pharmaceutically acceptable carriers and formulations containing these materials is described in, e.g., Remington's Pharmaceutical Sciences, 21st Edition, ed. University of the Sciences in Philadelphia, Lippincott, Williams & Wilkins, Philadelphia, PA, 2005. Examples of physiologically acceptable carriers include saline, glycerol, DMSO, buffers such as phosphate buffers, citrate buffer, and buffers with other organic acids; antioxidants including ascorbic acid; low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEENTM (ICI, Inc.; Bridgewater, New Jersey), polyethylene glycol (PEG), and PLURONICS™ (BASF; Florham Park, NJ). To provide for the administration of such dosages for the desired therapeutic treatment, compositions disclosed herein can advantageously comprise between about 0.1% and 99% by weight of the total of one or more of the subject compounds based on the weight of the total composition including carrier or diluent.

The term “increased” or “increase” as used herein generally means an increase by a statically significant amount; for the avoidance of any doubt, “increased” means an increase of at least 10% as compared to a reference level, for example an increase of at least about 20%, or at least about 30%, or at least about 40%, or at least about 50%, or at least about 60%, or at least about 70%, or at least about 80%, or at least about 90%, or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

As used herein “immune checkpoint inhibitor” or “checkpoint inhibitor” refers to a molecule that completely or partially reduces, inhibits, interferes with or modulates one or more checkpoint proteins. Checkpoint proteins include, but are not limited to, PD-1, PD-L1 and CTLA-4. Control samples (untreated with inhibitors) are assigned a relative activity value of 100%. In some embodiments, inhibition of a described target protein is achieved when the activity value relative to the control is about 80% or less, 50% or less, 25% or less, 10% or less, 5% or less, or 1% or less.

“Progression-free survival” or “PFS” refers to the length of time during and after the treatment of a disease, such as cancer, that a subject lives with the disease but it does not get worse. Progression-free survival may include the amount of time a subject has experienced a complete response or a partial response, as well as the amount of time a subject has experienced stable disease.

The term “reduced”, “reduce”, “suppress”, or “decrease” as used herein generally means a decrease by a statistically significant amount. However, for avoidance of doubt, “reduced” means a decrease by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (i.e. absent level as compared to a reference sample), or any decrease between 10-100% as compared to a reference level.

The term “subject” is defined herein to include animals such as mammals, including, but not limited to, primates (e.g., humans), cows, sheep, goats, horses, dogs, cats, rabbits, rats, mice and the like. In some embodiments, the subject is a human.

The terms “treat,” “treating,” “treatment,” and grammatical variations thereof as used herein, include partially or completely delaying, alleviating, mitigating or reducing the intensity of one or more attendant symptoms of a disorder or condition and/or alleviating, mitigating or impeding one or more causes of a disorder or condition. In some embodiments, treatments according to the invention are applied pallatively or remedially after development of a cancer. In some embodiments, the administration to a subject is prior to onset (e.g., before obvious signs of cancer) or during early onset (e.g., upon initial signs and symptoms of cancer). Prophylactic administration can occur for several days to years prior to the manifestation of symptoms of a disease (e.g., a cancer).

“Therapeutically effective amount” or “therapeutically effective dose” of a composition refers to an amount that is effective to achieve a desired therapeutic result. In some embodiments, a desired therapeutic result is a reduction of tumor size. In some embodiments, a desired therapeutic result is a reduction of cancer metastasis. In some embodiments, a desired therapeutic result is a reduction in amount or size of a skin cancer. In some embodiments, a desired therapeutic result is a reduction in amount or size of a melanoma. In some embodiments, a desired therapeutic result is the prevention of cancer relapse. In some embodiments, a desired therapeutic result is a reduction in an immune related adverse event. Therapeutically effective amounts of a given therapeutic agent will typically vary with respect to factors such as the type and severity of the disorder or disease being treated and the age, gender, and weight of the subject. The term can also refer to an amount of a therapeutic agent, or a rate of delivery of a therapeutic agent (e.g., amount over time), effective to facilitate a desired therapeutic effect, such as control of tumor growth. The precise desired therapeutic effect will vary according to the condition to be treated, the tolerance of the subject, the agent and/or agent formulation to be administered (e.g., the potency of the therapeutic agent, the concentration of agent in the formulation, and the like), and a variety of other factors that are appreciated by those of ordinary skill in the art. In some instances, a desired biological or medical response is achieved following administration of multiple dosages of the composition to the subject over a period of days, weeks, or years.

### METHODS

Disclosed herein is a method of treating a cancer in a subject comprising administering to the subject a therapeutically effective amount of a fecal sample and an anti-PD-1 antibody, wherein the fecal sample is derived from a donor that is responsive to a same or different anti-PD-1 antibody.

As used herein “responsiveness” refers to a beneficial outcome associated with an immune checkpoint inhibitor administration, such as a treatment of a cancer as compared to a control. In some embodiments, the beneficial outcome is a decrease or slowing in tumor growth, a decrease in tumor volume or size, a decrease in tumor number, a decrease in cancer recurrence, and/or a decrease in cancer metastasis, all as compared to a control subject or control or study population. In some embodiments, a donor responsive to the anti-PD-1 antibody has an ongoing durable partial response or complete response to the anti-PD-1 antibody. In some embodiments, the duration of the partial response of the responsive donor is more than 12 months, more than 13 months, more than 14 months, more than 15 months, more than 16 months, more than 17 months, more than 18 months, more than 19 months, more than 20 months, more than 21 months, more than 22 months, more than 23 months, more than 24 months, more than 26 months, more than 28 months, more than 30 months, more than 34 months, more than three years, more than four years, more than five years, or more than 10 years. In some embodiments, the duration of the complete response of the responsive donor is more than 6 months, more than 7 months, more than 8 months, more than 9 months, more than 10 months, more than 11 months, more than 12 months, more than 13 months, more than 14 months, more than 15 months, more than 16 months, more than 17 months, more than 18 months, more than 19 months, more than 20 months, more than 21 months, more than 22 months, more than 23 months, or more than 24 months.

It should be understood that “complete response” or “CR” as used herein means the disappearance of all signs of cancer (e.g., disappearance of all target lesions) in response to treatment. This does not mean the cancer has been cured. As used herein, “partial response” or “PR” refers to a decrease in the size of one or more tumors or lesions, or in the amount of cancer in the body, in response to treatment. For example, in some embodiments, PR refers to at least a 30% decrease in the sum of the longest diameters (SLD) of target lesions, taking as reference the baseline SLD. In some embodiments, the terms “objective response”, “complete response”, “partial response”, “progressive disease”, “stable disease”, “progression free survival”, “duration of response”, as used herein, are as defined and assessed by the investigators using RECIST v1.1 (Eisenhauer et al, Eur J of Cancer 2009; 45(2):228-47).

In some embodiments, the cancer is a melanoma. In some embodiments, the cancer is a metastatic melanoma or an advanced melanoma. In some embodiments the cancer is a refractory immune checkpoint inhibitor cancer, and in some embodiments, a refractory anti-PD-1 cancer. In some embodiments, the cancer is a refractory anti-PD-1 melanoma. As used herein, the word “refractory” refers to a disease or condition that is unresponsive to treatment or insufficiently responsive to treatment.

In some embodiments, the subject is less responsive to the anti-PD-1 antibody than the donor. In some embodiments, the subject is non-responsive to the anti-PD-1 antibody.

In some embodiments, the donor has been diagnosed with a melanoma. In some embodiments, the donor has had a progression-free survival (PFS) of at least about 12 months (for examples, at least 14 months, at least 18 months, at least 24 months, at least 30 months, at least 36 months, at least 42 months, or at least 48 months.

In some embodiments, the subject is administered an anti-PD-L1 and/or an anti-CTLA-4 antibody in addition to the anti-PD-1 antibody. In some embodiments, the donor has been administered an anti-PD-L1 and/or an anti-CTLA-4 antibody in addition to a same or different anti-PD-1 antibody that is administered to the subject. In some embodiments, the subject is administered the same anti-PD-1 antibody as was administered to the donor. In some embodiments, the subject is administered a different anti-PD-1 antibody as was administered to the donor.

As used herein, the term “PD-1 inhibitor” refers to a composition that binds to PD-1 and reduces or inhibits the interaction between the bound PD-1 and PD-L1. In some embodiments, the PD-1 inhibitor is an anti-PD-1 antibody. In some embodiments, the PD-1 inhibitor is a monoclonal antibody that is specific for PD-1 and that reduces or inhibits the interaction between the bound PD-1 and PD-L1. Non-limiting examples of anti-PD1 antibody are pembrolizumab, nivolumab, and cemiplimab. In some embodiments, the pembrolizumab is KEYTRUDA® or a bioequivalent. In some embodiments, the pembrolizumab is that described in U.S. Pat. Nos. 8,952,136, 8,354,509, or 8,900,587, all of which are incorporated by reference in their entireties. In some embodiments, the pembrolizumab has the Unique Ingredient Identifier (UNII) of the U.S. Food and Drug Administration of DPT0O3T46P. In some embodiments, the nivolumab is OPDIVO® or a bioequivalent. In some embodiments, the nivolumab has the Unique Ingredient Identifier (UNII) of the U.S. Food and Drug Administration of 31YO63LBSN. In some embodiments, the nivolumab is that described in U.S. Pat. Nos. 7,595,048, 8,738,474, 9,073,994, 9,067,999, 8,008,449, or 8,779,105, all of which are incorporated by reference in their entireties. In some embodiments, the cemiplimab is LIBTAYO® or a bioequivalent. In some embodiments, the cemiplimab has the Unique Ingredient Identifier (UNII) of the U.S. Food and Drug Administration of 6QVL057INT. In some embodiments, the cemiplimab is that described in U.S. Pat. No. 10,844,137, which is incorporated by reference in its entirety.

The term “PD-L1 inhibitor” refers to a composition that binds to PD-L1 and reduces or inhibits the interaction between the bound PD-L1 and PD-1. In some embodiments, the PD-L1 inhibitor is an anti-PD-L1 antibody. In some embodiments, the anti-PD-L1 antibody is a monoclonal antibody that is specific for PD-L1 and that reduces or inhibits the interaction between the bound PD-L1 and PD-1. Non-limiting examples of PD-L1 inhibitors are atezolizumab, avelumab and durvalumab. In some embodiments, the atezolizumab is TECENTRIQ® or a bioequivalent. In some embodiments, the atezolizumab has the Unique Ingredient Identifier (UNII) of the U.S. Food and Drug Administration of 52CMIOWC3Y. In some embodiments, the atezolizumab is that described in U.S. Pat. No. 8,217,149, which is incorporated by reference in its entirety.In some embodiments, the avelumab is BAVENCIO® or a bioequivalent. In some embodiments, the avelumab has the Unique Ingredient Identifier (UNII) of the U.S. Food and Drug Administration of KXG2PJ551I. In some embodiments, the avelumab is that described in U.S. Pat. App. Pub. No. 2014321917, which is incorporated by reference in its entirety. In some embodiments, the durvalumab is IMFINZI® or a bioequivalent. In some embodiments, the durvalumab has the Unique Ingredient Identifier (UNII) of the U.S. Food and Drug Administration of 28X28X9OKV. In some embodiments, the durvalumab is that described in U.S. Pat. No. 8,779,108, which is incorporated by reference in its entirety.

The term “CTLA-4 inhibitor” refers to a composition that binds to CTLA-4 and reduces or inhibits the interaction between the bound CTLA-4 and CD80/86. In some embodiments, the CTLA-4 inhibitor is an anti-CTLA-4 antibody. In some embodiments, the anti-CTLA-4 antibody is a monoclonal antibody that is specific for CTLA-4 and that reduces or inhibits the interaction between the bound CTLA-4 and CD80/86. In some embodiments, the anti-CTLA-4 antibody is ipilimumab. In some embodiments, the ipilimumab is Yervoy® or a bioequivalent. In some embodiments, the ipilimumab has the Unique Ingredient Identifier (UNII) of the U.S. Food and Drug Administration of 6T8C155666. In some embodiments, the ipilimumab is that described in U.S. Pat. No. 6,984,720, which is incorporated by reference in its entirety.

In some embodiments, the administration of the fecal sample is concurrently with the administration of the anti-PD-1 antibody. In some embodiments, the administration of the fecal sample occurs within 1, 2, 3, 4, 5, 6, 7, 8, 9, 10, 15, 20, 25, 30, 35, 40, 45, 50, 55, 60, 75, 90, 105, 120 minutes, 3, 4, 5, 6, 7, 8, 9, 10, 11, 12, 15, 18, 24, 30, 36, 48, 60 hours, 3, 4, 5, 6, 7, 8, 9, 11, 12, 13, 14, 15, 16, 17, 18, 19, 20, 21, 22, 23, 24, 25, 26, 27, 28, 29, 30 days, 2, 3, 4, 5, 6, 7, 8, 9, 10, 11, 12 months or more after the anti-PD-1 antibody is administered to the subject. In some embodiments, the administration of the fecal sample occurs within 1, 2, 3, 4, 5, 6, 7, 8, 9, 15, 20, 25, 30, 35, 40, 45, 50, 55, 60, 75, 90, 105, 120 minutes, 3, 4, 5, 6, 7, 8, 9, 10, 11, 12, 18, 24, 30, 36, 48, 60 hours, 3, 4, 5, 6, 7, 8, 9, 10, 11, 12, 13, 14, 15, 16, 17, 18, 19, 20, 21, 22, 23, 24, 25, 26, 27, 28, 29, 30 days, 2, 3, 4, 5, 6, 7, 8, 9, 10, 11, 12 months or more before the anti-PD-1 antibody is administered to the subject.

The fecal sample can be administered at least once, at least twice, at least three times, at least four times, at least five times, at least 10 times, at least 15 times, at least 20 times, or at least 50 times. In some embodiments, the method further comprises one or more further administrations of the anti-PD-1 antibody.

In some embodiments, the fecal sample obtained from the donor comprises a higher level of phylum Actinobacteria and/or phylum Firmicutes in comparison to a control. In some embodiments, the bacteria of phylum Firmicutes is a Lachnospiraceae, a Ruminococcaceae, and/or a Erysipelotrichaceae. In some embodiments, the bacteria of phylum Actinobacteria is a Bifidobacteriaceae and/or a Coriobacteriaceae. In some embodiments, the fecal sample obtained from the donor comprises a lower level of phylum Bacteroides (e.g., a Tannerellaceae or a Bacteroidaeceae) in comparison to a control. In some embodiments, the fecal sample obtained from the donor comprises a lower level of phylum Proteobacteria (e.g., a Sutterellaceae). The term “control” in these embodiments refers to a level in detected in a subject in general or a study population.

The methods provided herein can increases a level of bacteria of phylum Firmicutes and/or phylum Actinobacteria in the subject's gut in comparison to a control. In some embodiments, the bacteria of phylum Firmicutes is a Lachnospiraceae, a Ruminococcaceae, and/or a Erysipelotrichaceae. In some embodiments, the bacteria of phylum Actinobacteria is a Bifidobacteriaceae and/or a Coriobacteriaceae.

The methods provided herein can decreases a level of bacteria of phylum Bacteroides in the subject's gut in comparison to a control. In some embodiments, the bacteria of phylum Bacteroides is a Tannerellaceae and/or a Bacteroidaeceae.

It should be understood and herein contemplated that the methods provided herein can increase the effectiveness of an anti-PD-1 therapy in a subject having cancer. Accordingly, in some aspects, disclosed herein is a method of treating a PD-1-refractory melanoma in a subject comprising administering to the subject a therapeutically effective amount of a fecal sample and an anti-PD-1 antibody, wherein the fecal sample is derived from a donor that is responsive to a same or different anti-PD-1 antibody.

In some of these embodiments, the subject has been treated with an anti-PD-1 antibody prior to administration of the fecal sample and anti-PD-1 antibody. In some embodiments, the subject is less responsive to the anti-PD-1 antibody than the donor or nonresponsive to the anti-PD-1 antibody. In some embodiments, an anti-PD-L1 antibody and/oror an anti-CTLA-4 antibody is administered in addition to the anti-PD-1 antibody.

In some of these embodiments, the donor has been diagnosed with a melanoma. In some embodiments, the donor has had a progression-free survival (PFS) of at least about 12 months (for examples, at least 14 months, at least 18 months, at least 24 months, at least 30 months, at least 36 months, at least 42 months, or at least 48 months.

In some of these embodiments, the administration of the fecal sample is concurrent with the administration of the anti-PD-1 antibody. In some embodiments, the administration of the fecal sample occurs within 1, 2, 3, 4, 5, 6, 7, 8, 9, 10, 15, 20, 25, 30, 35, 40, 45, 50, 55, 60, 75, 105, 120 minutes, 3, 4, 5, 6, 7, 8, 9, 10, 11, 12, 15, 18, 24, 30, 36, 48, 60 hours, 3, 4, 5, 6, 7, 8, 9, 10, 11, 12, 13, 14, 15, 16, 17, 18, 19, 20, 21, 22, 23, 24, 25, 26, 27, 28, 29, 30 days, 2, 3, 4, 6, 7, 8, 9, 10, 11, 12 months or more after the anti-PD-1 antibody is administered to the subject. In some embodiments, the administration of the fecal sample occurs within 1, 2, 3, 4, 5, 6, 7, 8, 9, 10, 15, 20, 25, 30, 35, 40, 45, 50, 55, 60, 75, 90, 105, 120 minutes, 3, 4, 5, 6, 7, 8, 9, 11, 12, 15, 18, 24, 30, 36, 48, 60 hours, 3, 4, 5, 6, 7, 8, 9, 10, 11, 12, 13, 14, 15, 16, 17, 18, 19, 20, 21, 22, 23, 24, 25, 26, 27, 28, 29, 30 days, 2, 3, 4, 5, 6, 7, 8, 9, 10, 11, 12 months or more before the anti-PD-1 antibody is administered to the subject.

The fecal sample can be administered at least once, at least twice, at least three times, at least four times, at least five times, at least 10 times, at least 15 times, at least 20 times, or at least 50 times. In some embodiments, the method further comprises one or more further administrations of the anti-PD-1 antibody.

In some of these embodiments, the fecal sample obtained from the donor comprises a higher level of phylum Actinobacteria and/or phylum Firmicutes in comparison to a control. In some embodiments, the bacteria of phylum Firmicutes is a Lachnospiraceae, a Ruminococcaceae, and/or a Erysipelotrichaceae. In some embodiments, the bacteria of phylum Actinobacteria is a Bifidobacteriaceae and/or a Coriobacteriaceae. In some embodiments, the fecal sample obtained from the donor comprises a lower level of phylum Bacteroides (e.g., a Tannerellaceae or a Bacteroidaeceae) in comparison to a control. In some embodiments, the fecal sample obtained from the donor comprises a lower level of phylum Proteobacteria (e.g., a Sutterellaceae). The term “control” refers to a level detected in a subject in general or a study population.

It should also be understood that the foregoing relates to preferred embodiments of the present invention and that numerous changes may be made therein without departing from the scope of the invention. The invention is further illustrated by the following examples, which are not to be construed in any way as imposing limitations upon the scope thereof. On the contrary, it is to be clearly understood that resort may be had to various other embodiments, modifications, and equivalents thereof, which, after reading the description herein, may suggest themselves to those skilled in the art without departing from the spirit of the present invention and/or the scope of the appended claims. All patents, patent applications, and publications referenced herein are incorporated by reference in their entirety for all purposes.

### EXAMPLES

### Example 1

### Materials and Methods

Clinical Trial Design Summary, Recipient Eligibility Criteria, Donor Selection Criteria, Donor/Recipient Seromatching, and Study Conduct.

Summary: This was a phase II Simon's two-stage single-center study of concurrent fecal microbiota transplantation (FMT) with pembrolizumab in patients with programmed cell death protein 1 (PD-1)-refractory melanoma. The primary objective was to investigate whether a combination of single PD-1 responder (R)-derived FMT with pembrolizumab can convert PD-1 non-responders (NRs) to Rs. Secondary and exploratory objectives were to determine the effect of FMT administration on the composition and function of T cells and innate/adaptive immune system subsets and the gut microbiota, respectively.

Recipient Eligibility Criteria: Recipient eligibility was based upon prior exposure to PD-1 inhibitor therapy and response at first (or subsequent) restaging scan(s). Patients must have received a minimum of two cycles of anti-PD-1 to be considered eligible. Patients who received either nivolumab or pembrolizumab as a single agent or in combination with other standard or investigational agent(s) were eligible. PD-1-refractory disease was defined as progressive disease (PD) at first (or subsequent) radiographic evaluation while receiving PD-1 inhibitor treatment as assessed by response evaluation criteria in solid tumors (RECIST) v1.1 (E. A. Eisenhauer ET AL., 2009). Patients with stable disease (SD) as their best response were eligible, but patients with complete response (CR) or partial response (PR) as their best response were ineligible (H. M. Kluger et al., 2020). Other eligibility criteria included absence of active central nervous system (CNS) disease, presence of disease amenable to biopsy, and lack of Donor Selection Criteria: Candidate donors were patients with advanced unresectable stage IIIB-D or metastatic melanoma treated with anti-PD-1 (nivolumab or pembrolizumab) with ongoing durable PR or CR. Median duration of follow up was ≥24 months (for partial Rs) or ≥12 months (for complete Rs). Other eligibility criteria included willingness to complete a donor-specific questionnaire and undergo donor-specific serologic testing to evaluate infectious agents. Donor-specific exclusion criteria included history of antibiotic treatment during the 1 month preceding donation; history of intrinsic gastrointestinal illnesses including inflammatory bowel disease, irritable bowel syndrome, chronic diarrheal disorder (celiac disease), active primary gastrointestinal malignancies, or major gastrointestinal surgical procedures; history of symptomatic autoimmune illness; history of documented chronic pain syndromes (fibromyalgia, chronic fatigue) or neurologic, neurodevelopmental disorders; and history of metabolic syndrome, obesity (body mass index >35), or moderate-to-severe malnutrition (as assessed clinically). Only patients deemed suitable by a screening questionnaire and serological/stool/nasal swab tests were deemed suitable to donate stool samples to create FMTs. While on study, candidate FMT donors underwent systematic retesting before FMT sampling to minimize the possibility of transmitting infectious agents (FIG. 6). contraindication to FMT.

Donor/Recipient Seromatching and Study Conduct: Potential recipients underwent a screening evaluation consisting of imaging (including CNS if clinically suspected), tumor biopsy, and serological/stool studies to confirm suitability for FMT administration. Donors or recipients who tested positive for infections with latent potential (human immunodeficiency virus, hepatitis B/C, human T-cell lymphotropic virus type 1 [HTLV-1], HTLV-2, strongyloides, syphilis) and/or had evidence of multi-drug resistant organisms such as vancomycin-resistant Enterococcus, carbapenem-resistant Enterobacteriaceae, and extended spectrum beta-lactamase were ineligible. Candidate recipients and donors were sero-matched for cytomegalovirus, Epstein-Barr virus, herpes simplex virus 1/2, John Cunningham virus, human herpesvirus 6, and methicillin-resistant Staphylococcus aureus.

Eligible patients received FMT endoscopically with one cycle of pembrolizumab (±3 days) followed by three additional cycles of pembrolizumab (cycles 2-4), depending on which restaging computed tomography (CT) scans were performed. Patients with SD and/or responding disease continued to receive pembrolizumab on study until disease progression or intolerable toxicity for up to 2 years from FMT administration (35 cycles total). Response was first assessed after four cycles of pembrolizumab and every 12 weeks thereafter based on RECIST 1.1 (E. A. Eisenhauer et al., 2009). Because tumor pseudo-progression is a well-recognized consequence of PD-1-based immunotherapies and use of FMT as a therapeutic agent in cancer is highly novel, PD was confirmed if observed on two consecutive assessments of response at least 4 weeks apart, during which time pembrolizumab was continued.

DNA Extraction and Shotgun Metagenomic Sequencing and Analysis.

Total metagenomic DNA was extracted from stool samples using the MO BIO PowerSoil DNA Isolation Kit (MO BIO Laboratories, Carlsbad, CA, USA) and Epmotion 5075 liquid handling robot (Eppendorf). The DNA library was prepared using the Nextera DNA Flex Library Prep Kit, quantified using Qbit, and sequenced on the NovaSeq System (Illumina, Inc, San Diego, CA, USA) using the 2×150 base pair (bp) paired-end protocol.

For each shotgun metagenomic sample, after quality trimming and adapter clipping with Trimmomatic 0.36 (A. M. Bolger et al., 2014), raw reads were aligned against the human genome to filter out human reads with Bowtie2 v2.3.2 (B. Langmead et al., 2012). Leftover (non-host) reads were assembled using MEGAHIT v1.2.9 (D. Li et al., 2015). Resulting assembly contigs <500 bp were discarded. For the 216 samples sequenced, the mean number of non-human bp used for assembly into contigs was 2.73 Gbp±0.78 Gbp, yielding a mean assembly rate of 78.27%±7.48%.

Contigs were classified taxonomically by k-mer analysis using Kraken2 (D. E. Wood et al., 2019), with a custom 96-Gb Kraken2 database built with draft and complete genomes of all bacteria, archaea, fungi, viruses, and protozoa available in the NCBI GenBank in April 2020, in addition to human and mouse genomes. Contigs were annotated ab initio with Prokka v1.14.6 (T. Seemann 2014). Then reads used for assembly were aligned back to the assembly contigs to gauge sequencing depth of each contig. Unassembled reads were retrieved and classified one by one using Kraken2 on the same database. Taxonomic classifications were expressed as the last known taxon (LKT), which is the lowest unambiguous classification known for the query sequence, using Kraken's confidence scoring threshold of 5e-06 (using the —confidence parameter).

In each sample, relative abundance for each LKT was calculated by dividing the number of bp covering all contigs and unassembled reads classified as that LKT by the total number of host-filtered bp used for assembly in that sample. This ratio was multiplied by 10⁶ to yield relative abundance in parts per million (PPM).

For beta analysis, ordination plots were done using t-distributed stochastic neighbor embedding (t-SNE) implemented via the uwot package in R (github.com/jlmelville/uwot) and the ggplot2 library. Heatmaps were drawn using the ComplexHeatmap package for R (Z. Gu et al., 2016).

All codes used for shotgun sequencing analysis can be found within the in-house JAMS_BW package, version 1.5.5, publicly available on GitHub (github.com/johnmcculloch/JAMS_BW).

Meta-analysis of microbiome associated with the response was done as follows. Individual Rs were first analyzed using the non-parametric t-test. Then p-values and ratios were combined using Fisher's method and R package meta (github.com/guido-s/meta). Resultant data were visualized using the cladogram feature from package LEfSe (N. Segata et al., 2011).

Assessing Reactivity of Recipient Sera to Donor Fecal Bacteria by IgG Flow Cytometry

Evaluation of IgG Response in Recipient Sera to Donor Fecal Bacteria: Donor fecal bacteria were separated from particulates by centrifugation at 50 g for 1 minute. Fecal bacterial suspensions were normalized to equivalent OD600 and were incubated with recipient sera at 1:200 dilutions in phosphate buffered saline with 1% bovine serum albumin for 30 minutes. Suspensions were washed and stained with anti-IgG (PE, Miltenyi clone Is11-3B2.2.3; 1:50 dilution) and SYTO-62 (staining bacterial cells in APC; 1:1,000 dilution) for 15 minutes at 40 C. Suspensions were washed, fixed in 2% paraformaldehyde for 15 minutes, and analyzed on a Becton Dickinson Fortessa flow cytometer.

IgG Flow Cytometry Data Analysis: Change in IgG positivity of donor fecal bacteria for each FMT recipient was calculated in relation to the first FMT recipient serum sample available (baseline) compared to the subsequent two time points up to 50 days later. Analysis was limited to the subsequent two time points post-baseline because later time points were not equally available for all subjects due to mortality. Percent IgG-positive donor fecal bacteria were assessed by setting IgG-positive gates for negative control (no serum) donor bacterial suspension samples to 0.5% positivity. Area-under-the-curve (AUC, R package ‘DescTools’) for the change in percent IgG-positive donor bacteria was calculated adjusting all recipient time points relative to the baseline time point by subtraction, with the baseline IgG-positive proportion being thus set to zero. Student's T-test was performed to test differences in IgG positivity of donor fecal bacteria over time as quantified by AUC.

Multiparameter Flow Cytometry and Unsupervised Analysis of Peripheral Blood Mononuclear Cells. Cryopreserved peripheral blood mononuclear cells (PBMCs) from patients undergoing combined anti-PD-1 and FMT treatment at three time points (days 0, 21, and 42) were thawed, washed, and resuspended in complete Iscove's Dulbecco's Modified Eagle Medium (10% human serum, 1% penicillin and streptomycin, 1% L-glutamine, 1% Hepes, and 1% non-essential amino acids). Cells were equally divided into five staining panels (depicted below). Cells in each panel were labeled for viability with Zombie NIR (BioLegend, San Diego, CA, USA) (15 min, room temperature) and stained with 29-color panels of anti-human monoclonal antibodies against surface (20 min, 4° C.) and intracellular markers (30 min, 4° C.). Cells were permeabilized in 2% hypertonic formaldehyde for 20 min at room temperature, followed by 1×BD perm/wash buffer (BD Biosciences, Franklin Lakes, NJ, USA), per the manufacturer's protocol. Each panel included a set of markers of interest and a common core of lineage markers. The antibodies used are outlined in the table below and include: CD1a and CXCR-5 BUV395, CD16 BUV496, CD123 and CD25 BUV563, CD56 BUV661, CD19 and CD8 BUV737, CD14 and CD127 BUV805 (BD Biosciences), CD86 and TIGIT BV421 (BD Biosciences), IgD, CD27 and ICOS SuperBright436 (Thermo Fisher Scientific, Waltham, MA, USA), CD27, HLA-DR and Helios Pacific Blue, CD4OL and TCRva2 BV480, ICOS-L, CD45RA, CD28, NKp46 BV510, CD33 and CD19 BV570, BDCA-2 and Tim-3 BV605, Lag-3, CD103 and NKp30 BV650, Tim-3, CCR8, CD101 and CD127 BV711, CD3 Alexa532, CD15, CD96, T-bet, 2B4 and BTLA PerCPCy5.5 (BioLegend), PD-L1, CD39, Eomes and 4-1BB PerCPeFluor710 (BioLegend), CD112, CD226, TCF-1, BTLA, CD160 PE (BioLegend), CD155, CTLA-4 and TCRvα7.2 PE-Dazzle594 (BioLegend), HLA-DR, 4-1BB, CD161 and OX40 PE-Cy5 (BD Biosciences), CD68 and TCRγδ1 PE-Cy7 (BioLegend), VISTA, CRTAM, granzyme A, CXCL-13 APC (Thermo Fisher Scientific), CD38 APC/Cy5.5 (Thermo Fisher Scientific), CD11 c, granzyme B, NKG2A Alexafluor700 (BD Biosciences), CD112R Alexafluor700 (Biotechne), CD83, CCR7, perforin and CD57 APC/Fire750 (BioLegend). Spectral flow cytometry was carried out on a Cytek Aurora flow cytometer (Cytek Biosciences, Fremont, CA, USA). Supervised analysis was performed with FlowJo (BD Biosciences) for fine adjustments of channel spillovers and live cell subset extraction for unsupervised analysis. Unsupervised analysis was performed using the R Programming Language v3.6.0 with the CATALYST package v1.8.7, using a modified version of a previously published procedure (C. Krieg et al., 2018; M. Nowicka et al., 2017). Briefly, marker expression was transformed using arcsinh with a cofactor of 150. Samples were z-scored per batch to remove batch effects. Events that were more than five standard deviations away from the mean were removed. Events from all samples were clustered per panel and manually labeled based upon their mean fluorescence intensity (MFI) of lineage and differentiation markers. Clusters with the same label were combined. Visualization was performed using Uniform Manifold Approximation and Projection (UMAP) reduction. The frequency of cells for each sample was calculated by dividing the number of cells in a cluster by the total number of cells for that sample. To evaluate differences in cell frequency, unpaired t-tests were performed between the abundance of Rs and NRs for each cell type. For each cluster, unpaired t-tests were performed between the median expression of Rs and NRs.

TABLE 1
Antibodies Used for Multiparameter Spectral Flow Cytometry

| Fluoro- | | | | | |
|---|---|---|---|---|---|
| chrome | Panel 1 | Panel 2 | Panel 3 | Panel 4 | Panel 5 |
| BUV395 | CD1a | CXCR-5 | CXCR-5 | CXCR-5 | CXCR-5 |
| BUV496 | CD16 | CD16 | CD16 | CD16 | CD16 |
| BUV563 | CD123 | CD25 | CD25 | CD25 | CD25 |
| BUV661 | CD56 | CD56 | CD56 | CD56 | CD56 |
| BUV737 | CD19 | CD8 | CD8 | CD8 | CD8 |
| BUV805 | CD14 | CD127 | CD127 | CD14 | CD14 |
| BV421 | CD86 | TIGIT | TIGIT | TIGIT | TIGIT |
| Super Bright | IgD | CD27 | ICOS | CD27 | ICOS |
| 436 | | | | | |
| Pacific blue | CD27 | HLA-DR | Helios | HLA-DR | CD27 |
| BV480 | CD40 | TCRgd2 | TCRgd2 | TCRgd2 | TCRgd2 |
| BV510 | ICOS-L | CD45RA | CD28 | NKp46 | CD28 |
| BV570 | CD33 | CD19 | CD19 | CD19 | CD19 |
| BV605 | BDCA-2 | TIM-3 | TIM-3 | TIM-3 | TIM-3 |
| BV650 | Lag-3 | Lag-3 | CD103 | NKp30 | CD103 |
| BV711 | TIM-3 | CCR8 | CD101 | CD127 | CD127 |
| BV750 | CD11b | CD4 | CD4 | CD4 | CD4 |
| BV785 | CD141 | PD-1 | PD-1 | PD-1 | PD-1 |
| FITC | CD80 | Ki67 | Ki67 | Ki67 | Ki67 |
| Alexa flor | CD3 | CD3 | CD3 | CD3 | CD3 |
| 532 | | | | | |
| PercP-Cy 5.5 | CD15 | CD96 | T-bet | 2B4 | CD160 |
| PercP- | PD-L1 | CD39 | Eomes | PD-L1 | TCR |
| EFluor710 | | | | | aV7.2 |
| PE | CD112 | CD226 | TCF-1 | BTLA | CD161 |
| PE-Dazzle | PVR | CTLA-4 | TCRva7 | Blimp-1 | BTLA |
| 594 | | | | | |
| PE-Cy5 | HLA-DR | 4-1BB | CD161 | OX40 | 4-1BB |
| PE-Cy7 | CD68 | TCRgd1 | TCRgd1 | TCRgd1 | TCRgd1 |
| APC | VISTA | CRTAM | GRZ-A | CXCL-13 | Granzyme |
| | | | | | A |
| APC-Cy5.5 | CD38 | CD38 | CD38 | CD38 | CD38 |
| AlexaFlu- | CD11c | PVRIG | GRZB | NKG2A | Granzyme |
| or700 | | | | | B |
| Zombie NIR | Viability | Viability | Viability | Viability | Viability |
| APC-Fire750 | CD83 | CCR7 | Perforin | CD57 | Perforin |

Single-Cell RNA Sequencing of Tumor Samples.

Fluorescence-activated cell sorting-isolated CD45+ cells from tumor biopsies were processed using 10× Genomics' Chromium platform for droplet-based single-cell RNA sequencing (scRNA-seq). Eleven samples were collected at day 0, nine samples were collected around day 56, and one sample was collected at day 129. Gene expression libraries were generated using the Chromium Single Cell 5′ Library Construction Kit (v1.0 chemistry, PN-1000006) following the CG000086 user guide. Each library was sequenced on the Illumina NovaSeq 6000 System with a PE150 configuration to a target depth of 50 k read pairs per cell. Sequenced gene expression libraries were aligned to the GRCh38-2020-A reference genome using 10X Genomics' Cell Ranger count v4.0.0 with default settings. Cell count matrices were loaded into R and processed using the standard workflow of Seurat v3.2.0 (29). Feature counts were normalized using NormalizeData with default settings. This function divides the feature counts of each cell by the total counts for that cell, multiplied by 10,000, followed by taking the natural log. Then T and B cell V, D, J, and C genes were removed to prevent clustering by clonotype. Any gene with the following prefixes were removed: TRA(VDJC)-, TRB(VDJC)-, TRD(VDJC)-, TRG(VDJC)-, IGH(VDJC)-, IGL(VDJC)-, IGK(VDJC)-. Cells were removed in which the percentage of reads that aligned to the mitochondrial genome was greater than 10%. To exclude empty droplets and multiplets, cells with unique feature counts less than 200 or greater than 3000 were excluded. Any sample that had fewer than 300 cells after the previous quality control step was removed. Variable gene features were identified using Find VariableFeatures with default settings. Batch effects were removed using FindIntegrationAnchors and IntegrateData with default settings. Integrated expression data were scaled and centered using ScaleData with default settings. Clustering was performed using FindNeighbors and FindClusters using default settings. Each cluster was identified based upon gene expression. To facilitate this process, differential gene expression between each cluster and all other clusters was performed using FindMarkers with a min.pct=0.25. Two clusters that were identified as melanoma contamination (SPPlhigh, APODhigh) and one cluster that was likely dead or dying cells were removed. Clusters identified as the same cell type were merged. The top 50 genes for each cell type after this process are shown in Table 5. At this point, samples that contained fewer than 300 cells were removed because of potential bias they could introduce in abundance calculations. A UMAP projection was calculated using RunPCA and RunUMAP. Abundance for each sample was calculated by dividing the number of cells of a particular cell type by the total number of cells for that sample. Unpaired t-tests between Rs and NRs were performed for each cell type before and after (day 56) treatment. Uncorrected p-values were reported due to the low number of samples. Phenotype differences between Rs and NRs were calculated by only selecting cells from samples collected post-treatment (day 56) and running FindMarkers on each cell type between cells from Rs and NRs.

Cytokine/Chemokine Multiplex Analysis.

Serum samples were analyzed by Eve Technologies (Calgary, Canada) using the Human Cytokine Array/Chemokine Array 65-Plex Panel (HD65) [EGF, Eotaxin, FGF-2, Flt-3 Ligand, Fractalkine, G-CSF, GM-CSF, GRO, IFN-α2, IFN-γ, IL-10, IL-12 (p40), IL-12 (p70), IL-13, IL-15, IL-17A, IL-18, IL-1RA, IL-1α, IL-1β, IL-2, IL-3, IL-4, IL-5, IL-6, IL-7, IL-8, IL-9, IP-10, MCP-1, MCP-3, MDC (CCL22), MIP-1α, MIP-1β, PDGF-AA, PDGF-AB/BB, RANTES, TGFα, TNF, LTα, VEGF-A, sCD40L, Eotaxin-2, MCP-2, BCA-1, MCP-4, I-309, IL-16, TARC, 6CKine, Eotaxin-3, LIF, TPO, SCF, TSLP, IL-33, IL-20, IL-21, IL-23, TRAIL, CTACK, SDF-1, ENA-78, MIP-1d, IL-28A]. Data obtained were log2-transformed and quantile-normalized, and statistical tests were performed [principal component analysis (PCA), analysis of variance (ANOVA), t-test]. Data were analyzed and visualized using Partek Genomic suite 6.0 (Partek Inc., St. Louis, MO, USA).

Metabolome and Lipidome Analysis.

Samples were analyzed by Metabolon, Inc. (Durham, NC, USA). Serum samples were analyzed using liquid chromatography-tandem mass spectrometry and gas chromatography-mass spectrometry. Peaks were identified using Metabolon's proprietary chemical reference library. Resultant chemicals were mapped to known classes of biological molecules and metabolic pathways using the Kyoto Encyclopedia of Genes and Genomes database. Both lipidomic and metabolomic datasets were log2-transformed and quantile-normalized, and statistical tests were performed (PCA, ANOVA, t-test). Data were analyzed and visualized using Partek Genomic suite 6.0 (Partek Inc.).

Statistical Analyses.

Time to progression for recipients was defined as the interval (in months) from day 0 (FMT administration) to the date of radiographic progression. Patients who did not progress were censored at their date of last contact. Overall survival for recipients was defined as the interval (in months) from day 0 (FMT administration) to the date of death. Patients who were alive as of September 1, 2020 were censored at their date of last contact. Time to progression for FMT donors was defined as the interval (in months) from date of PD-1 therapy initiation to date of last contact.

Transkingdom Network Analysis of Multiomic Data

Network Reconstruction: To create a statistical model of robust interactions between the different players, we created a transkingdom network independent of a particular group or patient (R. R. Rodrigues et al., 2018). First, we identified elements (serum cytokines, serum metabolites, multi-parameter flow cytometry, and stool bacteria) that differentially changed upon FMT treatment in Rs compared with NRs. Next, Spearman rank correlation was calculated between all pairs of elements. To keep robust relationships, interactions not involving bacteria were selected if they had the same sign of correlation in: (i) NRs pre-FMT, (ii) Rs pre-FMT, (iii) NRs post-FMT, and (iv) Rs post-FMT. For within- and inter-omic interactions involving bacteria, only the post-FMT groups (iii and iv) were used to check for consistent signs of correlation. Additionally, pairs were included in the network if they satisfied principles of causality (i.e., satisfied fold-change relationship between the two partners in Rs vs. NRs post-FMT) (A. Yambartsev et al., 2016) and 5% combined p-value cutoff for meta-analysis of correlations based on Fisher's z transformation of correlations (metacor in R package meta v4.9-7). Finally, 1% false discovery rate (FDR) was used for edges involving bacteria or within edges (except flow), whereas 2.5% PDR was used for edges involving flow cytometry parameters or inter-omic edges, and edges involving metabolites also had 30% individual (within group) p-value cutoff.

Detecting Gene Expression Subnetworks: Cytoscape Software 2.6.3 was used to visualize the networks (43). To identify subnetworks in the transkingdom network, we used the MCODE v1.2 (Molecular Complex Detection) (G. D. Bader et al., 2003) plug-in for Cytoscape to identify clusters (subnetworks). The largest subnetwork containing nodes from all four different omics data was selected for further investigation.

### Example 2

### Results

Immune checkpoint blockade with monoclonal antibodies (mAbs) targeting programmed cell death protein 1 (PD-1) provides long-term clinical benefits to nearly 40% patients with advanced melanoma (J. Larkin et al., 2015; A. Ribas, et al., 2016; C. Robert et al., 2015; C. Robert et al., 2019; C. Robert et al., 2015). In addition to tumor-intrinsic mechanisms supporting resistance to anti-PD-1 mAbs (anti-PD-1), the gut microbiome is a major tumor-extrinsic regulator of responses to anti-PD-1 (A. Dzutsev et al., 2015; B. B. Finlay et al., 2020; R. S. Goldszmid et al., 2015; H. M. Zarour 2016). In mice, composition of the gut microbiome modulates therapeutic activity of anti-PD-1/programmed death-ligand 1 (PD-L1), and administration of certain gut commensals or fecal microbiota transplantation (FMT) promotes anti-PD-1 efficacy in melanoma-bearing mice (V. Gopalakrishnan et al., 2018; V. Matson et al., 2018; B. Routy et al., 2018). While multiple studies have reported that a favorable gut microbiome is associated with response to anti-PD-1 in cancer patients, its precise composition is not yet fully understood (V. Gopalakrishnan et al., 2018; V. Matson et al., 2018; B. Routy et al., 2018). Specifically in melanoma, key bacterial species belonging to various phyla including Actinobacteria (Bifidobacteriaceae spp., Coriobacteriaceae spp.) and Firmicutes (Ruminococcaceae spp., Lachnospiraceae spp.) are associated with favorable response to mAbs targeting PD-1 in cancer patients, although limited concordance among the identified species has been reported in different studies (V. Gopalakrishnan et al., 2018; V. Matson et al., 2018; B. Routy et al., 2018; A. E. Frankel et al., 2017; B. A. Peters et al., 2019). It has not been evaluated whether microbiome-based therapy can overcome resistance to anti-PD-1 in patients with advanced melanoma. To address this question, a single-arm clinical trial was designed to evaluate the safety and efficacy of FMT obtained from individual long-term responder (R) melanoma patients together with anti-PD-1 in PD-1-refractory metastatic melanoma patients (NCT03341143; FIG. 5).

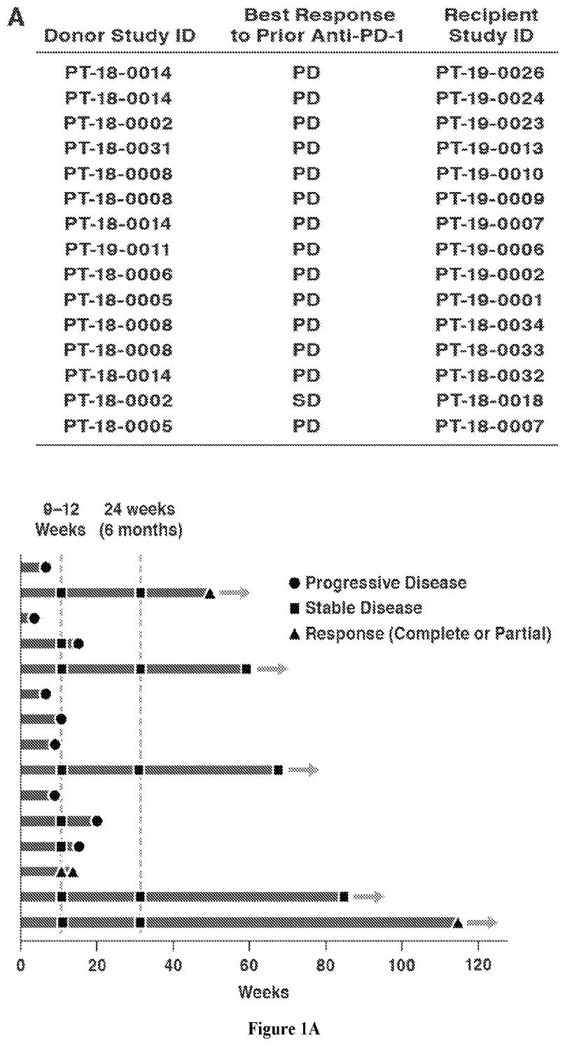

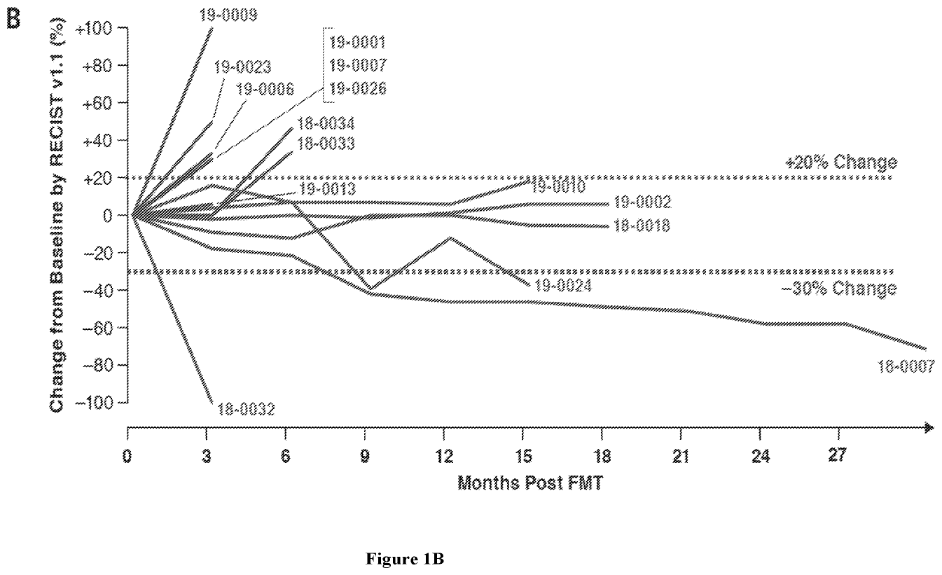

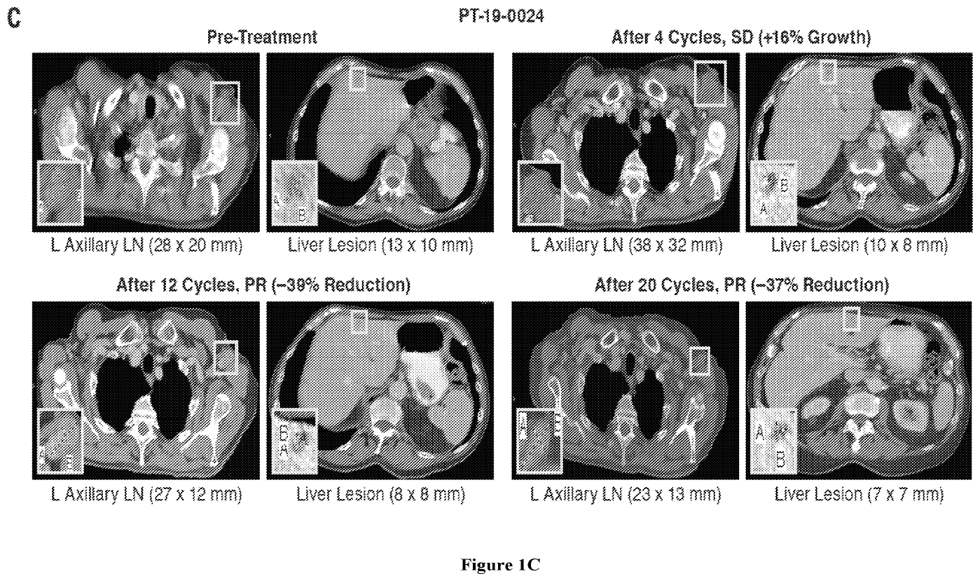

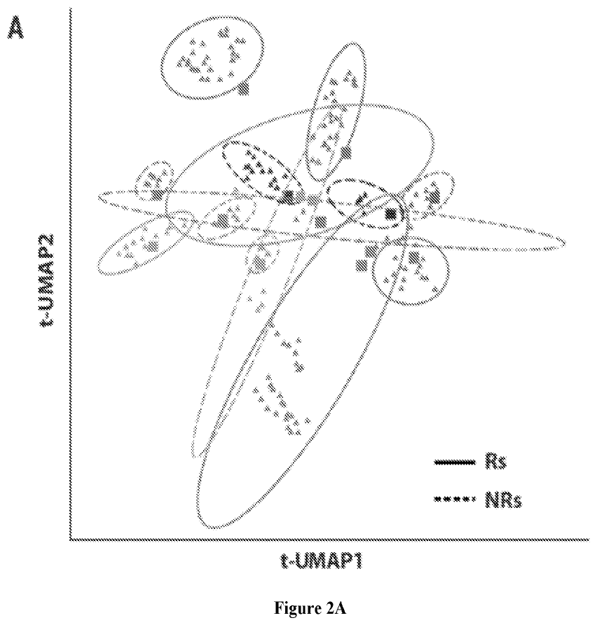

Sixteen melanoma patients were enrolled between June 2018 and January 2020 (Table 2), and the results presented herein reflect a data cutoff of Sep. 1,2020. All melanoma patients were primary refractory to anti-PD-1 therapy, defined as patients with no prior response to anti-PD-1 alone or in combination with anti-cytotoxic T-lymphocyte-associated protein 4 or investigational agents (Table 2), who had confirmed primary progressive disease (PD) as assessed using response evaluation criteria in solid tumors (RECIST v1.1) by an independent radiologist (E. A. Eisenhauer et al., 2009; H. M. Kluger et al., 2020). Among PD-1-refractory patients included in the trial, only one had a best response of short-term stable disease (SD) before radiographically confirmed PD. All enrolled patients and candidate donors underwent serial stool sampling and extensive infectious studies to eliminate the possibility of transmitting infectious agents (FIG. 6). Seven donors including four with complete response (CR) and three partial response (PR) with median progression-free survival (PFS) of 56 months (range: months) were used to treat 16 patients (Table 3). Blood and stool specimens were obtained serially and screened for 32 viral, bacterial, fungal, and protozoan agents before FMT (FIG. 6). A single donor-derived FMT was administered along with pembrolizumab (FIG. 5) followed by additional pembrolizumab therapy every 3 weeks until disease progression or intolerable toxicity. Radiographic assessments were conducted every 12 weeks (4 cycles), and response was classified using RECIST v1.1. Of the 16 patients enrolled, 15 received FMT and pembrolizumab and had at least one restaging computed tomography (CT) scan and thus were deemed evaluable for response. One patient who had a rapid clinical decline following FMT deemed secondary to rapid disease progression was evaluable for safety but not response. Objective responses (OR) were noted in three (PT-18-0032, CR; PT-18-0007 and PT-19-0024, PR) out of fifteen patients [objective response rate (ORR): 20%] while three (PT-18-0018, PT-19-0002, PT-19-0010) out of fifteen patients (20%) had durable SD lasting >12 months (FIGS. 1A and 1B). Representative radiographic examples from all 3 responders patients with ORR are provided (FIG. 1C and FIGS. 7 and 8). Median PFS and overall survival (OS) in all patients was 3.0 and 7.0 months, respectively, at a median follow-up of 7 months. In six patients with disease control (i.e., OR and SD), median PFS and OS was 14.0 and 14.0 months, respectively (FIG. 1B). Among these patients, one patient (PT-18-0007) exhibited ongoing PR after >2 years and is currently on surveillance, while four patients (PT-18-0018, PT-19-0002, PT-19-0010, PT-19-0024) remain on treatment. One patient (PT-18-0032) with radiographic CR underwent an elective surgical procedure for spinal stenosis but suffered a spinal infarct unrelated to therapy and subsequently passed away. This study shows that FMT together with anti-PD-1 overcame resistance to anti-PD-1 in a subset of PD-1-refractory melanoma patients. Although these preliminary findings warrant further evaluation in a larger randomized clinical trial, the observed ORR was superior to ORRs reported in melanoma patients primary refractory to anti-PD-1 therapy treated beyond progression (A. Ribas et al., 2018). Treatment-related adverse events (AEs) were minimal (Table 4). While all patients experienced at least one AE, these were mostly low-grade (Grade 1: 72.9%; Grade 2: 20.0%). Endocrinologic AEs, mostly hypothyroidism, occurred in 17.6% of patients and were easily managed with hormone replacement. Grade 3 AEs occurred in three patients: two instances of fatigue, in which underlying endocrinological issues were excluded and resolved, and one case of peripheral motor neuropathy (PT-19-0024) that required hospitalization, intravenous immunoglobulin, and corticosteroids and resolved with no further sequelae upon reinstitution of pembrolizumab.

To evaluate the effects of FMT on gut microbiota composition in recipients and the relationship with clinical response defined as OR or SD >12 months after FMT and anti-PD-1 based on RECIST 1.1 criteria, shotgun metagenomic sequencing was performed on 223 fecal samples obtained from recipients (n=15) and donors (n=7). For each recipient, one pre-FMT sample (obtained 7-21 days pre-FMT) and all available post-FMT samples (obtained weekly for 12 weeks and then every 3 weeks for as long as the patient remained on trial) were sequenced (FIG. 2A). For each corresponding FMT, the unique donor-specific “FMT infusate” obtained from specific FMT donors was sequenced (Table 2 and FIG. 9A). Uniform manifold approximation and projection (UMAP) analyses depicted distinct gut microbiota composition in recipients (FIG. 2A) and donors (FIG. 9A). No significant difference in response after FMT was observed in patients that received infusates from donors that had either a CR or PR to anti-PD1 (FIG. 9B). The microbiota in CR donors exhibited higher alpha-diversity than in PR donors but no significant differences were observed between donors and recipients before FMT (FIG. 9C). In recipients, gut microbiota composition changed following a single FMT in Rs and non-responders (NRs) (FIG. 2A), an alteration that persisted unless a significant perturbation occurred (e.g., antibiotic use in PT-18-0032 before day 22 and PT-18-0018 before day 84). Of note, all separately collected infusates produced from individual donors were highly similar to each other (FIGS. 9A and 9D). To evaluate intra-patient variability in both donors and recipients, sample variance was computed across all time points and all taxa in all available samples from recipients and donors (only those with ≥3 samples) (FIG. 2B). Variance was significantly greater in all recipients post-FMT compared to donors (FIG. 2B), although Rs had a trend towards greater variance compared to NRs (FIG. 10). Differences and rate of change in microbiome communities in recipients were quantified using multidimensional Euclidean distance. Specifically, the speed of traversion as inferred from the Euclidean distance traversed per day trended higher in Rs than NRs (FIG. 2C). Although this parameter did not reach statistical significance likely due to limited sample size, the Euclidean distance still notably separated patients who achieved disease control from those who did not.

To investigate the degree of donor microbial implantation and its relationship to response in patients over time, the acquired similarity of the recipient microbiota to the donor microbiota was evaluated by measuring the Euclidian distance between donor microbial composition and every available time point of the corresponding recipients, starting from pre-treatment sample (FIG. 2D). The gut microbiota composition shifted significantly towards donor microbiota in Rs but not in NRs (FIG. 2D and FIG. 11 left panels). FMT implantation was clearly heterogenous in the NR group (n=9), where about half of the recipients had samples that were similar, and the other half had samples that were dissimilar to corresponding donors (FIG. 2D, upper panel). In contrast, the gut microbiota in Rs (n=6) uniformly shifted towards donor samples (FIG. 2D, lower panel). The humoral immune response to donor bacteria was also evaluated using recipient serum and donor feces and observed that FMT administration induced donor bacteria-specific IgG to a greater extent in Rs compared to non-NRs (FIG. 2E and FIG. 12), indicating that successful implantation and mucosal translocation of the transplanted bacteria, known to induce an IgG response against commensal bacteria, may preferentially occur in Rs (M. Y. Zeng et al., 2016).

The use of systemic antibiotics was prohibited on study; however, PT-18-0018 developed a soft tissue infection requiring antibiotics (FIG. 13). During this period, pembrolizumab was withheld, although stool samples and imaging were obtained at protocol-specified intervals. Initial microbiome post-FMT shifted towards the donor's microbiome with SD. Following antibiotics, metagenomic data revealed striking changes in microbial community composition (loss of F. prausnitzi, Alistipes spp., and Ruminococcaceae spp.), which correlated with clinical progression. A second transplant from the same donor was performed nearly 1 year after initial FMT and resulted in rapid gut colonization associated with ongoing SD (FIG. 13). Overall, while successful colonization post-FMT did not always resensitize PD-1-refractory melanoma patients to anti-PD-1, clinical response was associated with FMT implantation and donor microbiota-specific IgG response. Further, the changes of microbiome composition post-FMT were rapidly disturbed by use of antibiotics, which depleted beneficial taxa (i.e., F. prausnitzi, Alistipes spp., and Ruminococcaceae spp.), in accordance with previously reported deleterious effects of antibiotic use with regards to anti-PD-1 in cancer patients (B. Routy et al., 2018; D. J. Pinato et al., 2019).

To analyze the bacterial species most commonly associated with clinical benefit to FMT, we first performed statistical analyses between pre- and post-FMT samples in Rs, followed by a meta-analysis using Fisher's method to identify common features among all samples (FIG. 2F and FIG. 14). Several bacterial species associated with clinical response have been previously reported (Bifidobacterium longum, Colinsella aerofaciens, Faecalibacterium prausnitzii) (V. Gopalakrishnan et al., 2018; V. Matson et al., 2018). The vast majority of significantly enriched taxa in Rs belonged to the phyla Firmicutes (Lachnospiraceae and Ruminococcaceae families) and Actinobacteria (Bifidobacteriaceae and Coriobacteriaceae families), while the majority of bacteria decreased in Rs belonged to phylum Bacteroidetes.

To evaluate the immunological effects of FMT and anti-PD-1 in treated patients, we performed multiparameter flow cytometry and single-cell RNA sequencing (scRNA-seq) analysis from peripheral blood mononuclear cells (PBMCs) and tumor samples, respectively, collected pre- and post-treatment. Unsupervised single-cell analysis on spectral flow cytometry of PBMCs was performed at three consecutive time points pre- and post-treatment (FIGS. 3A, 3B and FIGS. 15A, 15B) (C. Krieg et al., 2018; M. Nowicka et al., 2017). Compared with NRs, Rs displayed higher percentages of CD56⁺CD8⁺ T cells post-treatment (day 42), which represent a subset of activated CD8⁺ T cells with higher cytolytic functions (M. J. Pittet et al., 2000; T. Ohkawa et al., 2001; S. Guia et al., 2008) (FIG. 3B). CD8±CD56+T cells expressed high levels of TIGIT, CD57, 2B4, OX40, ICOS, 4-1BB, CD16, NKp46, NKp30, granzyme B, perforin, and CD103 compared with total CD8⁺ T cells (FIG. 15C). These findings are in line with previous reports in cancer patients responding to immunotherapy, including radio-immunotherapy (K. Hasumi et al., 2013) or PD-1 blockade (A. Ribas et al., 2016). In Rs, CD8⁺ T cells upregulated TIGIT (day 21) as well as T-bet and LAG-3 (day 42) and downregulated CD27 (day 21) compared with NRs at these timepoints (FIG. 3C). Rs exhibited lower percentages of naive CD8⁺ T cells (day 21) and higher percentages of TEMRA cells (day 42) compared with NRs (FIG. 3D). These observations indicate that circulating CD8+T cells are more activated and differentiated in Rs, as previously shown in cancer patients who respond to PD-1 blockade (A. O. Kamphorst et al., 2017; A. Kunert et al., 2019). Mucosal-associated invariant T (MAIT) cells, which respond to bacterial antigens and inflammatory cytokines, expressed more granzyme B (day 42) and less CD27 (day 42) in Rs compared with NRs, suggesting a more differentiated phenotype (FIG. 3E). No significant differences were observed in the frequencies of circulating myeloid cell subsets pre- and post-treatment (FIGS. 15D, 15E). Together, these data show that FMT together with anti-PD-1 expanded activated CD56⁺CD8⁺ T cells and increased activation of CD8⁺ T and MAIT cells in PBMCs of Rs.

CD45⁺ cells were sorted from single-cell suspension obtained from tumor biopsies used for scRNA-seq analysis (10× Genomics Chromium). In total, 64,340 cells from 17 tumor samples were collected pre- (4 Rs and 5 NRs) and post-treatment (2 Rs and 6 NRs). After normalization and batch effect removal (T. Stuart et al., 2019), cells were clustered into 26 groups. Each cluster was manually labeled by gene expression profile (Table 5) to identify 10 unique cell types (FIG. 3F). A high frequency of myeloid cells (p=0.026) and CD4⁺ regulatory T cells (p=0.02) was observed among CD45⁺ cells in NRs compared to Rs post-treatment, whereas the other clusters did not exhibit significant changes (FIG. 3G and FIG. 16). Myeloid cells expressed high levels of CXCL8 (IL-8) and SPP1 (osteopontin), which increased post-FMT in NRs compared with Rs, suggesting a myeloid gene signature previously associated with tumor progression (K. A. Schalper et al., 2020; H. Alshetaiwi et al., 2020;) (FIGS. 3H, 3I). CD8⁺ T cells upregulated HLA class II genes, CD74 and GZMK in Rs compared with NRs post-treatment, supporting increased T-cell activation at tumor sites (FIG. 3I, left panel). Together, our findings demonstrate that FMT and anti-PD-1 counteract myeloid-induced immunosuppression to augment CD8⁺T-cell activation in the tumor microenvironment of Rs. Insufficient tumor tissue was available to evaluate the role of tumor mutation burden, PD-L1 expression and IFN-γ gene expression signature in predicting clinical outcome.

To assess the impact of FMT on systemic parameters of the host and its relationship with response to therapy, multi-omics analysis of serum samples was performed including analyses of serum cytokines and chemokines, as well as serum metabolomics and lipidomics analyses. While Rs and NRs exhibited similar composition of serum cytokines and chemokines, Luminex multiparameter proteomics profiling showed a prominent shift in levels of circulating cytokines and chemokines in Rs, whereas NRs had little or no change (FIG. 4A, left principal component analysis panel). Multiple circulating cytokines and chemokines decreased post-FMT in Rs, including MCP1/CCL2, CXCL8/IL-8 and IL-18 that have been associated with negative outcomes to anti-PD-1 (B. Gok Yavuz et al., 2019; M. Terme et al., 2011), as well as IL-12p70 and IFN-γ (FIG. 4A, heatmap). These latter two cytokines are usually associated with anti-tumor effector T cell responses. However, chronic activation of the IL-12/IFN-γ axis, as may occur in anti-PD-1 treated refractory patients, can induce a multigenic resistance program in tumor cells and can disrupt T cell response and differentiation/exhaustion through production of nitric oxide and downregulation of TCF1 (J. L. Benci et al., 2016; M. Danilo et al., 2018; H. K. Koblish et al., 1998). The cytokines and chemokines that were most prominently upregulated are associated with follicular helper T and B-cell signatures found in tertiary lymphoid structures, such as IL-21 and CXCL13/BCP1. The data show upregulation of type II cytokines such as IL-IL-13, and IL-10; the TNF family of cytokines such as TNF and TRAIL; and cytokines promoting monocyte cell migration (FRACTALKINE) and DC expansion (FLT3L). Altogether, these findings show that Rs downregulated multiple circulating cytokines and chemokines associated with resistance to anti-PD-1 while upregulating circulating biomarkers associated with favorable clinical outcome. In particular, Rs exhibited decreased circulating IL-8 and decreased frequencies of IL-8-producing myeloid-cells in tumors. IL-8 is an immunosuppressive cytokine secreted by intratumoral and circulating myeloid cells, levels of which correlate adverse prognosis to anti-PD-1 in multiple cancers, including melanoma (K. A. Schalper et al., 2020; M F Sanmamed et al., 2017).

FMT and anti-PD-1 resulted in significant changes in the serum metabolomic profile of both Rs and NRs (FIG. 4B, FIG. 17); although the most dramatic metabolomic shifts occurred in Rs (FIG. 4B, PCA plot). The most significant and pronounced changes post-FMT affected metabolites usually associated with gut microbiota (P. Vernocchi et al., 2016). Serum bile acids were increased after FMT with more efficient transformation of primary to secondary bile acids in Rs versus NRs (FIG. 18). In addition, the levels of bacterial catabolism products of aromatic compounds through benzoate degradation were higher in Rs than in NRs. Some of these compounds including hyppurate, p-cresol sulfate, and hydrocinnamate have been described as biomarkers of microbiome diversity and correlated with the presence of taxa associated with response to anti-PD-1 (FIGS. 17 and 18) (T. Pallister et al., 2017). While pre-FMT samples from Rs and NRs had very similar lipidomic profiles, significant changes were observed in post-FMT samples from Rs but not NRs (FIG. 4C, FIG. 19) including higher levels of triacylglycerols and lower levels of monoacylglycerols and diacylglycerols, possibly reflecting microbiome-controlled differences in lipid absorption (K. Martinez-Guryn et al., 2018).

To identify causal relationships between host and microbes independent of a particular group or patient, a statistical model was created for the robust interactions between the different players—“transkingdom network” analyses—using different types of -omics data (R. R. Rodrigues et al., 2018; A. Yambartsev et al., 2016). The transkingdom network consisted of 371 nodes and 819 edges, where nodes represented individual elements of -omics datasets (metagenomic, metabolomic, lipidomic, flow cytometric, and proteomic) and edges represented positive and negative correlations between those elements (FIG. 4D). The vast majority of inter-omic edges belonged to the microbial metagenome dataset, which had a large number of positive (red) and negative (blue) network connections with the metabolomic and proteomic datasets. Microbial nodes were more densely connected to other -omics datasets than any other node, indicating their central role in governing inter-omic changes following FMT and anti-PD-1. Some nodes in the transkingdom network analysis were highly interconnected, forming subnetworks. These subnetworks were identified using the MCODE plugin of Cytoscape (P. Shannon et al., 2003), and one subnetwork connected all four -omics datasets (FIG. 4E). In particular, gut bacterial commensals previously shown to increase (Faecalibacterium prausnitzii and Akkermansia muciniphila) and decrease (Bacteroides genus) responses to anti-PD-1 (V. Gopalakrishnan et al., 20118) were negatively and positively correlated with CXCL8/IL-8, respectively.

In summary, these findings show that a single FMT administered colonoscopically together with PD-1 blockade successfully colonized the gut of Rs and reprogrammed the tumor microenvironment to overcome primary resistance to anti-PD-1 in a subset of patients with advanced melanoma. FMT shifted microbiome composition towards taxa favoring anti-PD-1 efficacy to induce clinical responses to anti-PD-1 in PD-1-refractory melanoma patients, who had an immunological ability to respond to the treatment but exhibited an unfavorable microbiota composition. Conversely, PD-1-refractory patients do not respond to FMT for various reasons including: a) inability to respond to the tumor regardless of microbiota composition due to the patient's immunodeficient status or lack of tumor imunogenicity, b) absence of taxa needed for anti-PD-1 therapy effectiveness in the FMT, c) failure of the FMT to successfully implant into the recipient and induce perturbations of host microbiota favoring anti-PD-1.

TABLE 2
Baseline Patient Demographics and
Clinical Characteristics (n = 15*)

| Characteristic | Value | |
|---|---|---|
| Median age, years (range) | 61 | (35-85) |
| Sex | | |
| Male | 11 | (73.3%) |
| Female | 4 | (26.7%) |
| Caucasian ethnicity | 15 | (100.0%) |
| Median BMI (range)^{#} | 27.7 | (21.9-55.3) |
| Neutrophil to lymphocyte ratio (NLR)^{#} | | |
| Pre-treatment, median (range) | 3.4 | (1.4-6.3) |
| Post-treatment, median (range) | 3.9 | (1.9-17.3) |
| ECOG performance status | | |
| 0 | 12 | (80.0%) |
| 1 | 3 | (20.0%) |
| Primary site | | |
| Cutaneous | 13 | (86.7%) |
| Mucosal | 1 | (6.7%) |
| Unknown primary | 2 | (13.3%) |
| Extent of disease | | |
| Cutaneous, subcutaneous and/or lymph node | 6 | (40.0%) |
| metastases | | |
| Lung metastases | 2 | (13.3%) |
| Visceral metastases (excluding CNS) | 5 | (33.3%) |
| Visceral metastases (including treated CNS disease) | 2 | (13.3%) |
| LDH level, (IU/L) | | |
| Normal | 1 | (6.7%) |
| 1-2x ULN (171) | 11 | (73.3%) |
| >2x ULN | 3 | (20.0%) |
| Mutation status | | |
| BRAF | 4 | (26.7%) |
| NRAS | 3 | (20.0%) |
| WT | 8 | (53.3%) |
| Number of prior therapies for metastatic disease | | |
| Median | 2 | |
| 1 | 8 | (53.3%) |
| 2 | 4 | (26.7%) |
| ≥3 | 3 | (20.0%) |

*Sixteen patients were enrolled, of whom 15 were evaluable (defined as having started therapy andhad ≥1 restaging scan). One patient (PT-18-0017) who experienced rapid clinical decline shortly after one cycle of therapy secondary to disease progression was not included in efficacy and microbiome analyses but was evaluable for safety.
^{#}Testing the effects of BMI, NLR on microbial composition and clinical response revealed no significant effect upon either compositional similarity or diversity in recipients using logistic andlinear regression.

TABLE 3
Baseline Patient Demographics and Clinical Characteristics of FMT Donors

| | | | | Number of | | | | |
|---|---|---|---|---|---|---|---|---|
| | | | Stage | Systemic | Best | Current | | |
| 17-034 | | Age | (before | Therapies | Response | Response | | |
| Donor | | (at | anti- | Before | to Anti- | to Anti- | PFS | Current |
| ID | Sex | LFU) | PD-1) | Anti-PD-1 | PD-1* | PD-1* | (months) | Status |
| PT-18- | M | 75 | IV-A | 1 | CR | CR | 60 | On |
| 0002 | | | | | | | | surveillance |
| PT-18- | F | 66 | IV-C | 1 | CR | CR | 62 | On |
| 0005 | | | | | | | | surveillance |
| PT-18- | F | 66 | IV-C | 0 | CR | CR | 56 | On |
| 0006 | | | | | | | | surveillance |
| PT-18- | M | 74 | IV-A | 0 | PR | PR | 56 | On |
| 0008 | | | | | | | | surveillance |
| PT-18- | M | 62 | IV-A | 0 | PR | PR | 45 | On |
| 0014 | | | | | | | | surveillance |
| PT-18- | F | 71 | IV-B | 0 | PR | PR | 70 | On |
| 0031 | | | | | | | | surveillance |
| PT-19- | M | 77 | IV-D | 0 | CR | CR | 43 | On |
| 0011 | | | | | | | | surveillance |
| PT-18- | M | 75 | IV-A | 1 | CR | CR | 60 | On |
| 0002 | | | | | | | | surveillance |

*Response was assessed using RECIST v1.1 by an independent blinded radiologist

TABLE 4
Summary of Adverse Events in the FMT/Pembrolizumab
Clinical Trial (n = 16)

| | | | |
|---|---|---|---|
| Grade | Grade | Grade | Grade |
| 1 | 2 | 3 | 4/5 |
| (N, %) | (N, %) | (N, %) | (N, %) |

Constitutional

| | | | | | | |
|---|---|---|---|---|---|---|
| Fever | 1 (1.6) | 0 | (0.0) | 0 | (0.0) | 0 (0.0) |
| Fatigue | 6 (9.7) | 5 | (29.4) | 2 | (33.3%) | 0 (0.0) |
| Anorexia | 2 (3.2) | 1 | (5.9) | 0 | (0.0) | 0 (0.0) |
| Weight loss | 2 (3.2) | 1 | (5.9) | 0 | (0.0) | 0 (0.0) |

Hematologic

| | | | | | | |
|---|---|---|---|---|---|---|
| Anemia | 0 (0.0) | 1 | (5.9) | 0 | (0.0) | 0 (0.0) |
| Lymphocyte count | 1 (1.6) | 0 | (0.0) | 0 | (0.0) | 0 (0.0) |
| decrease | | | | | | |
| Platelet count decrease | 1 (1.6) | 0 | (0.0) | 0 | (0.0) | 0 (0.0) |

Endocrine

| | | | | | | |
|---|---|---|---|---|---|---|
| Adrenal insufficiency | 0 (0.0) | 0 | (0.0) | 0 | (0.0) | 0 (0.0) |
| Hypothyroidism | 3 (4.8) | 3 | (17.6) | 0 | (0.0) | 0 (0.0) |

Dermatologic

| | | | | | | |
|---|---|---|---|---|---|---|
| Rash | 1 (1.6) | 0 | (0.0) | 0 | (0.0) | 0 (0.0) |
| Vitiligo | 1 (1.6) | 0 | (0.0) | 0 | (0.0) | 0 (0.0) |
| Pruritus | 1 (1.6) | 1 | (5.9) | 0 | (0.0) | 0 (0.0) |

Renal and Electrolyte

| | | | | | | |
|---|---|---|---|---|---|---|
| Creatinine increase | 1 (1.6) | 0 | (0.0) | 0 | (0.0) | 0 (0.0) |
| BUN increase | 3 (4.8) | 0 | (0.0) | 0 | (0.0) | 0 (0.0) |
| Hypokalemia | 1 (1.6) | 0 | (0.0) | 0 | (0.0) | 0 (0.0) |
| Hyponatremia | 10 (16.1) | 0 | (0.0) | 1 | (16.7) | 0 (0.0) |
| Hypophosphatemia | 2 (3.2) | 1 | (5.9) | 0 | (0.0) | 0 (0.0) |
| Hyperphosphatemia | 2 (3.2) | 0 | (0.0) | 0 | (0.0) | 0 (0.0) |

Gastrointestinal

| | | | | | | |
|---|---|---|---|---|---|---|
| Bloating | 14 (22.6) | 2 | (11.8) | 0 | (0.0) | 0 (0.0) |
| Nausea and vomiting | 1 (1.6) | 1 | (5.9) | 0 | (0.0) | 0 (0.0) |
| Aspartate | 2 (3.2) | 0 | (0.0) | 0 | (0.0) | 0 (0.0) |
| aminotransferase | | | | | | |
| increase | | | | | | |
| Alanine | 2 (3.2) | 0 | (0.0) | 0 | (0.0) | 0 (0.0) |
| aminotransferase | | | | | | |
| increase | | | | | | |
| Alkaline phosphatase | 1 (1.6) | 0 | (0.0) | 0 | (0.0) | 0 (0.0) |
| increase | | | | | | |
| Total bilirubin increase | 1 (1.6) | 0 | (0.0) | 0 | (0.0) | 0 (0.0) |
| Albumin decrease | 2 (3.2) | 0 | (0.0) | 0 | (0.0) | 0 (0.0) |
| Diarrhea (non-colitis) | 1 (1.6) | 1 | (5.9) | 0 | (0.0) | 0 (0.0) |

Neurologic

| | | | | | | |
|---|---|---|---|---|---|---|
| Fatigue | 0 (0.0) | 0 | (0.0) | 2 | (33.3) | 0 (0.0) |
| Peripheral motor | 0 (0.0) | 0 | (0.0) | 1 | (16.7) | 0 (0.0) |
| neuropathy | | | | | | |

TABLE 5
Top 50 Differentially Expressed Genes in Combined Clusters in scRNA-seq Analyses

| | | CD4 T | CD4 T | | | | | | Mast |
|---|---|---|---|---|---|---|---|---|---|
| CD4 T | CD8 T | Regs | FH | T FOS | NK | B | Myeloid | pDC | Cells |
| IL7R | CCL5 | FOXP3 | CXCL13 | EGR1 | GNLY | CD79A | CST3 | PTGDS | TPSAB1 |
| CCR7 | CD8A | BATF | NMB | GADD45B | FGFBP2 | IGKC | LYZ | PLD4 | TPSB2 |
| ANXA1 | GZMK | CARD16 | NR3C1 | JUN | SPON2 | MS4A1 | IFI30 | SOX4 | CPA3 |
| S1PR1 | LAG3 | TIGIT | TOX2 | TNF | PRF1 | HLA-DRA | S100A8 | LILRA4 | HPGD |
| KLF2 | CD8B | IL32 | IGFBP4 | FOS | GZMB | CD83 | SPP1 | IRF7 | CLU |
| LEF1 | GZMA | TNFRSF4 | ITM2A | CITED2 | KLRF1 | HLA-DQB1 | TIMP1 | TCF4 | HPGDS |
| TCF7 | GZMH | TNFRSF18 | CD200 | HSPA1B | KLRD1 | CD74 | CXCL8 | IRF8 | CD9 |
| LTB | NKG7 | IL2RA | MAGEH1 | IER2 | FCGR3A | HLA-DRB5 | APOE | GPR183 | GATA2 |
| FLT3LG | CST7 | TBC1D4 | DUSP4 | FOSB | NKG7 | HLA-DQA1 | C1QC | SERPINF1 | VWA5A |
| GIMAP7 | DUSP2 | CTLA4 | TNFRSF4 | UBE2S | FCER1G | HLA-DPA1 | C1QB | ITM2C | PRG2 |
| MAL | CCL4 | ARID5B | FKBP5 | DUSP1 | KLRB1 | MEF2C | S100A9 | IL3RA | TPSD1 |
| AQP3 | PTMS | CXCR6 | PDCD1 | ID2 | CTSW | IGHM | C1QA | NPC2 | LTC4S |
| NOSIP | KLRK1 | LINC01943 | TSHZ2 | H2AFX | HOPX | FCER2 | TYROBP | GZMB | SLC18A2 |
| LDHB | RGS1 | ICOS | GK | SNHG12 | CLIC3 | HLA-DRB1 | FTL | CCDC50 | HDC |
| CD55 | COTL1 | RTKN2 | RNF19A | NR4A1 | XCL2 | CD19 | FCER1G | CST3 | CTSG |
| RPL32 | CRTAM | GLRX | CORO1B | DNAJB1 | EFHD2 | CD79B | AIF1 | APP | KIT |
| RPS12 | FABP5 | PBXIP1 | LIMS1 | IER3 | CCL4 | HLA-DPB1 | PLAUR | SPIB | BACE2 |
| TPT1 | PDCD1 | LAYN | TOX | NEU1 | TRDC | HERPUD1 | CTSL | IRF4 | RHEX |
| ARRDC2 | ITM2A | LTB | PASK | IER5 | ADGRG1 | VPREB3 | IFITM3 | JCHAIN | AL157895.1 |
| FXYD5 | APOBEC3G | STAM | CD4 | SERTAD1 | CX3CR1 | ID3 | CD68 | SEC61B | IL1RL1 |
| RPL5 | IFNG | PIM2 | SH2D1A | RSRP1 | SH2D1B | SMIM14 | PSAP | UGCG | SIGLEC6 |
| SELL | CD27 | IKZF2 | MAF | SRSF7 | XCL1 | NR4A1 | CD14 | TSPAN13 | RAB32 |
| SATB1 | TNFSF9 | CORO1B | ZNRF1 | JUNB | CD247 | HLA-DMA | CTSB | SCT | PTGS1 |
| RPS3A | LYST | RGS1 | SPOCK2 | PPP1R15A | S1PR5 | CD37 | CCL2 | PLAC8 | RHOBTB3 |
| RPS13 | GZMB | TNFRSF1B | PHACTR2 | UBB | AREG | BASP1 | C15orf48 | CLIC3 | CNRIP1 |
| EEF1A1 | CD3D | AC017002.3 | FAAH2 | H3F3B | CST7 | SPIB | FCN1 | MPEG1 | MAOB |
| KLF3 | DUSP5 | S100A4 | ICOS | ZFP36L1 | CCL3 | HVCN1 | SERPINA1 | GRN | GCSAML |
| CAMK4 | PLAAT4 | GBP5 | SRGN | HSPA1A | IL2RB | LY9 | CXCL2 | SMPD3 | STXBP6 |
| RPL3 | CD2 | UGP2 | TNIK | SAP18 | KLRC1 | ARHGAP24 | SPI1 | TPM2 | GMPR |
| GAS5 | CYTOR | CYTOR | ARID5B | TUBA1B | MATK | RALGPS2 | G0S2 | TCL1A | NTRK1 |
| RPL34 | CTSW | DUSP4 | COTL1 | HSPA8 | PLAC8 | NCF1 | HLA-DRA | CYB561A3 | TIMP3 |
| EEF1B2 | CXCR6 | IL12RB2 | LAT | FUS | MAFF | CD40 | SOD2 | LRRC26 | SLC45A3 |
| RPS6 | PRF1 | DNPH1 | FYN | EIF4A3 | CMC1 | CXCR5 | IER3 | TRAF4 | RGS13 |
| RPL13 | HCST | CD2 | SESN3 | HIST2H2AA4 | GZMH | LY86 | CXCL3 | CD74 | HS3ST1 |
| RPS14 | TENT5C | GK | HNRNPLL | HEXIM1 | IFITM2 | SNX2 | NPC2 | DERL3 | PMP22 |
| RPL14 | CMC1 | SPOCK2 | TNFRSF18 | EIF4A2 | TTC38 | CD22 | IFI27 | FCER1G | FCER1G |
| RPL36 | CLEC2B | DUSP16 | IL6ST | CD69 | CEBPD | LYN | LST1 | MZB1 | TMEM176B |
| SARAF | IL32 | NCF4 | TCF7 | MARCKSL1 | FGR | EZR | HLA-DRB1 | CLEC4C | ALOX5 |
| RPL4 | CD3E | HLA-A | TBC1D4 | MYLIP | FOSL2 | SYNGR2 | S100A11 | EGLN3 | CST3 |
| GIMAP5 | RUNX3 | B2M | FYB1 | DNAJA1 | MAP3K8 | BLK | SAT1 | LDLRAD4 | RAB27B |
| GPR183 | TIGIT | HTATIP2 | BATF | SRSF2 | TBX21 | FCRLA | CCL3 | TYROBP | IFITM3 |
| RPL11 | CXCR3 | CASP1 | CTLA4 | TUBB2A | PTGDR | ADAM28 | GRN | LILRB4 | ACSLA |
| RPS3 | IDH2 | RPS27L | LINC00963 | BRD2 | CD300A | LINC00926 | IL1RN | RNASE6 | STX3 |
| EEF1G | PTPRCAP | CTSC | CNIH1 | TUBA1A | ITGB2 | CD24 | CTSD | RASD1 | BTK |
| RPLP0 | GZMM | FAS | SARAF | TXNIP | PLEK | SWAP70 | MS4A6A | PTCRA | ABCC4 |
| RPS8 | RAB27A | GBP2 | SOD1 | SNHG5 | ABHD17A | HSP90AB1 | HLA-DPA1 | KRT5 | GLUL |
| RPL9 | SH2D1A | MAF | PLIN2 | PNP | KLRK1 | FCRL1 | MAFB | C12orf75 | HES1 |
| RPL36A | SRRT | SOD1 | TGIF1 | HSP90AA1 | GZMA | HLA-DMB | HLA-DPB1 | GAS6 | SLC44A1 |
| RPL22 | FYN | TMEM173 | SLA | SNHG1 | GZMM | AFF3 | BCL2A1 | DNASE1L3 | SOX4 |

### REFERENCES

1. J. Larkin, C. D. Lao, W. J. Urba, D. F. McDermott, C. Horak, J. Jiang, J. D. Wolchok,

Efficacy and Safety of Nivolumab in Patients With BRAF V600 Mutant and BRAF Wild-Type Advanced Melanoma: A Pooled Analysis of 4 Clinical Trials. JAMA Oncol 1, 433-440 (2015).
2. A. Ribas, O. Hamid, A. Daud, F. S. Hodi, J. D. Wolchok, R. Kefford, A. M. Joshua, A. Patnaik, W. J. Hwu, J. S. Weber, T. C. Gangadhar, P. Hersey, R. Dronca, R. W. Joseph, H. Zarour, B. Chmielowski, D. P. Lawrence, A. Algazi, N. A. Rizvi, B. Hoffner, C. Mateus, K. Gergich, J. A. Lindia, M. Giannotti, X. N. Li, S. Ebbinghaus, S. P. Kang, C. Robert, Association of Pembrolizumab With Tumor Response and Survival Among Patients With Advanced Melanoma. JAMA 315, 1600-1609 (2016).
3. C. Robert, G. V. Long, B. Brady, C. Dutriaux, M. Maio, L. Mortier, J. C. Hassel, P. Rutkowski, C. McNeil, E. Kalinka-Warzocha, K. J. Savage, M. M. Hernberg, C. Lebbe, J. Charles, C. Mihalcioiu, V. Chiarion-Sileni, C. Mauch, F. Cognetti, A. Arance, H. Schmidt, D. Schadendorf, H. Gogas, L. Lundgren-Eriksson, C. Horak, B. Sharkey, I. M. Waxman, V. Atkinson, P. A. Ascierto, Nivolumab in previously untreated melanoma without BRAF mutation. N Engl J Med 372, 320-330 (2015).
4. C. Robert, A. Ribas, J. Schachter, A. Arance, J. J. Grob, L. Mortier, A. Daud, M. S. Carlino, C. M. McNeil, M. Lotem, J. M. G. Larkin, P. Lorigan, B. Neyns, C. U. Blank, T. M. Petrella, 0. Hamid, S. C. Su, C. Krepler, N. Ibrahim, G. V. Long, Pembrolizumab versus ipilimumab in advanced melanoma (KEYNOTE-006): post-hoc 5-year results from an open-label, multicentre, randomised, controlled, phase 3 study. Lancet Oncol 20, 1239-1251 (2019).
5. C. Robert, J. Schachter, G. V. Long, A. Arance, J. J. Grob, L. Mortier, A. Daud, M. S. Carlino, C. McNeil, M. Lotem, J. Larkin, P. Lorigan, B. Neyns, C. U. Blank, O. Hamid, C. Mateus, R. Shapira-Frommer, M. Kosh, H. Zhou, N. Ibrahim, S. Ebbinghaus, A. Ribas, K.-. investigators, Pembrolizumab versus Ipilimumab in Advanced Melanoma. N Engl J Med 372, 2521-2532 (2015).
6. A. Dzutsev, R. S. Goldszmid, S. Viaud, L. Zitvogel, G. Trinchieri, The role of the microbiota in inflammation, carcinogenesis, and cancer therapy. Eur J Immunol 45, 17-31 (2015).
7. B. B. Finlay, R. Goldszmid, K. Honda, G. Trinchieri, J. Wargo, L. Zitvogel, Can we harness the microbiota to enhance the efficacy of cancer immunotherapy? Nat Rev Immunol 20, 522-528 (2020).
8. R. S. Goldszmid, A. Dzutsev, S. Viaud, L. Zitvogel, N. P. Restifo, G. Trinchieri,

Microbiota modulation of myeloid cells in cancer therapy. Cancer Immunol Res 3, 103-109 (2015).
9. H. M. Zarour, Reversing T-cell Dysfunction and Exhaustion in Cancer. Clin Cancer Res 22, 1856-1864 (2016).
10. V. Gopalakrishnan, C. N. Spencer, L. Nezi, A. Reuben, M. C. Andrews, T. V. Karpinets, P. A. Prieto, D. Vicente, K. Hoffman, S. C. Wei, A. P. Cogdill, L. Zhao, C. W. Hudgens, D. S. Hutchinson, T. Manzo, M. Petaccia de Macedo, T. Cotechini, T. Kumar, W. S. Chen, S. M. Reddy, R. Szczepaniak Sloane, J. Galloway-Pena, H. Jiang, P. L. Chen, E. J. Shpall, K. Rezvani, A. M. Alousi, R. F. Chemaly, S. Shelburne, L. M. Vence, P. C. Okhuysen, V. B. Jensen, A. G. Swennes, F. McAllister, E. Marcelo Riquelme Sanchez, Y. Zhang, E. Le Chatelier, L. Zitvogel, N. Pons, J. L. Austin-Breneman, L. E. Haydu, E. M. Burton, J. M. Gardner, E. Sirmans, J. Hu, A. J. Lazar, T. Tsujikawa, A. Diab, H. Tawbi, I. C. Glitza, W. J. Hwu, S. P. Patel, S. E. Woodman, R. N. Amaria, M. A. Davies, J. E. Gershenwald, P. Hwu, J. E. Lee, J. Zhang, L. M. Coussens, Z. A. Cooper, P. A. Futreal, C. R. Daniel, N. J. Ajami, J. F. Petrosino, M. T. Tetzlaff, P. Sharma, J. P. Allison, R. R. Jenq, J. A. Wargo, Gut microbiome modulates response to anti-PD-1 immunotherapy in melanoma patients. Science 359, 97-103 (2018).
11. V. Matson, J. Fessler, R. Bao, T. Chongsuwat, Y. Zha, M. L. Alegre, J. J. Luke, T. F. Gajewski, The commensal microbiome is associated with anti-PD-1 efficacy in metastatic melanoma patients. Science 359, 104-108 (2018).
12. B. Routy, E. Le Chatelier, L. Derosa, C. P. M. Duong, M. T. Alou, R. Daillere, A. Fluckiger, M. Messaoudene, C. Rauber, M. P. Roberti, M. Fidelle, C. Flament, V. Poirier-Colame, P. Opolon, C. Klein, K. Iribarren, L. Mondragon, N. Jacquelot, B. Qu, G. Ferrere, C. Clemenson, L. Mezquita, J. R. Masip, C. Naltet, S. Brosseau, C. Kaderbhai, C. Richard, H. Rizvi, F. Levenez, N. Galleron, B. Quinquis, N. Pons, B. Ryffel, V. Minard-Colin, P. Gonin, J. C. Soria, E. Deutsch, Y. Loriot, F. Ghiringhelli, G.

Zalcman, F. Goldwasser, B. Escudier, M. D. Hellmann, A. Eggermont, D. Raoult, L. Albiges, G. Kroemer, L. Zitvogel, Gut microbiome influences efficacy of PD-1-based immunotherapy against epithelial tumors. Science 359, 91-97 (2018).
13. A. E. Frankel, L. A. Coughlin, J. Kim, T W. Froehlich, Y. Xie, E. P. Frenkel, A. Y. Koh, Metagenomic Shotgun Sequencing and Unbiased Metabolomic Profiling Identify Specific Human Gut Microbiota and Metabolites Associated with Immune Checkpoint Therapy Efficacy in Melanoma Patients. Neoplasia 19, 848-855 (2017).
14. B. A. Peters, M. Wilson, U. Moran, A. Pavlick, A. Izsak, T. Wechter, J. S. Weber, I. Osman, J. Ahn, Relating the gut metagenome and metatranscriptome to immunotherapy responses in melanoma patients. Genome Med 11, 61 (2019).
15. E. A. Eisenhauer, P. Therasse, J. Bogaerts, L. H. Schwartz, D. Sargent, R. Ford, J. Dancey, S. Arbuck, S. Gwyther, M. Mooney, L. Rubinstein, L. Shankar, L. Dodd, R. Kaplan, D. Lacombe, J. Verweij, New response evaluation criteria in solid tumours: revised RECIST guideline (version 1.1). Eur J Cancer 45, 228-247 (2009).
16. H. M. Kluger, H. A. Tawbi, M. L. Ascierto, M. Bowden, M. K. Callahan, E. Cha, H. X. Chen, C. G. Drake, D. M. Feltquate, R. L. Ferris, J. L. Gulley, S. Gupta, R. W. Humphrey, T. M. LaVallee, D. T. Le, V. M. Hubbard-Lucey, V. A. Papadimitrakopoulou, M. A. Postow, E. H. Rubin, E. Sharon, J. M. Taube, S. L. Topalian, R. Zappasodi, M. Sznol, R. J. Sullivan, Defining tumor resistance to PD-1 pathway blockade: recommendations from the first meeting of the SITC Immunotherapy Resistance Taskforce. J Immunother Cancer 8, (2020).
17. A. Ribas, J. M. Kirkwood, K. T. Flaherty, Anti-PD-1 antibody treatment for melanoma. Lancet Oncol 19, e219 (2018).
18. M. Y. Zeng, D. Cisalpino, S. Varadarajan, J. Hellman, H. S. Warren, M. Cascalho, N. Inohara, G. Nunez, Gut Microbiota-Induced Immunoglobulin G Controls Systemic Infection by Symbiotic Bacteria and Pathogens. Immunity 44, 647-658 (2016).
19. D. J. Pinato, S. Howlett, D. Ottaviani, H. Urus, A. Patel, T. Mineo, C. Brock, D. Power, O. Hatcher, A. Falconer, M. Ingle, A. Brown, D. Gujral, S. Partridge, N. Sarwar, M. Gonzalez, M. Bendle, C. Lewanski, T. Newsom-Davis, E. Allara, M. Bower, Association of Prior Antibiotic Treatment With Survival and Response to Immune Checkpoint Inhibitor Therapy in Patients With Cancer. JAMA Oncol 5, 1774-1778 (2019).
20. C. Krieg, M. Nowicka, S. Guglietta, S. Schindler, F. J. Hartmann, L. M. Weber, R. Dummer, M. D. Robinson, M. P. Levesque, B. Becher, High-dimensional single-cell analysis predicts response to anti-PD-1 immunotherapy. Nat Med 24, 144-153 (2018).
21. M. Nowicka, C. Krieg, H. L. Crowell, L. M. Weber, F. J. Hartmann, S. Guglietta, B. Becher, M. P. Levesque, M. D. Robinson, CyTOF workflow: differential discovery in high-throughput high-dimensional cytometry datasets. F1000Res 6, 748 (2017).
22. M. J. Pittet, D. E. Speiser, D. Valmori, J. C. Cerottini, P. Romero, Cutting edge: cytolytic effector function in human circulating CD8+T cells closely correlates with CD56 surface expression. J Immunol 164, 1148-1152 (2000).
23. T. Ohkawa, S. Seki, H. Dobashi, Y. Koike, Y. Habu, K. Ami, H. Hiraide, I. Sekine, Systematic characterization of human CD8+ T cells with natural killer cell markers in comparison with natural killer cells and normal CD8+ T cells. Immunology 103, 281-290 (2001).
24. S. Guia, C. Cognet, L. de Beaucoudrey, M. S. Tessmer, E. Jouanguy, C. Berger, O. Filipe-Santos, J. Feinberg, Y. Camcioglu, J. Levy, S. Al Jumaah, S. Al-Hajjar, J. L. Stephan, C. Fieschi, L. Abel, L. Brossay, J. L. Casanova, E. Vivier, A role for interleukin-12/23 in the maturation of human natural killer and CD56+ T cells in vivo. Blood 111, 5008-5016 (2008).
25. K. Hasumi, Y. Aoki, R. Wantanabe, D. L. Mann, Clinical response of advanced cancer patients to cellular immunotherapy and intensity-modulated radiation therapy. Oncoimmunology 2, e26381 (2013).
26. A. Ribas, D. S. Shin, J. Zaretsky, J. Frederiksen, A. Cornish, E. Avramis, E. Seja, C. Kivork, J. Siebert, P. Kaplan-Lefko, X. Wang, B. Chmielowski, J. A. Glaspy, P. C. Tumeh, T. Chodon, D. Pe'er, B. Comin-Anduix, PD-1 Blockade Expands Intratumoral Memory T Cells. Cancer Immunol Res 4, 194-203 (2016).
27. A. O. Kamphorst, R. N. Pillai, S. Yang, T. H. Nasti, R. S. Akondy, A. Wieland, G. L. Sica, K. Yu, L. Koenig, N. T. Patel, M. Behera, H. Wu, M. McCausland, Z. Chen, C. Zhang, F. R. Khuri, T. K. Owonikoko, R. Ahmed, S. S. Ramalingam, Proliferation of PD-1+ CD8 T cells in peripheral blood after PD-1-targeted therapy in lung cancer patients. Proc Natl Acad Sci USA 114, 4993-4998 (2017).
28. A. Kunert, E. A. Basak, D. P. Hurkmans, H. E. Balcioglu, Y. Klaver, M. van Brakel, A. A. M. Oostvogels, C. H. J. Lamers, S. Bins, S. L. W. Koolen, A. A. M. van der Veldt, S. Sleijfer, R. H. J. Mathijssen, J. Aerts, R. Debets, CD45RA(+)CCR7(−) CD8 T cells lacking co-stimulatory receptors demonstrate enhanced frequency in peripheral blood of NSCLC patients responding to nivolumab. J Immunother Cancer 7, 149 (2019).
29. T. Stuart, A. Butler, P. Hoffman, C. Hafemeister, E. Papalexi, W. M. Mauck, 3rd, Y. Hao, M. Stoeckius, P. Smibert, R. Satija, Comprehensive Integration of Single-Cell Data. Cell 177, 1888-1902.e1821 (2019).
30. K. A. Schalper, M. Carleton, M. Zhou, T. Chen, Y. Feng, S. P. Huang, A. M. Walsh, V. Baxi, D. Pandya, T. Baradet, D. Locke, Q. Wu, T. P. Reilly, P. Phillips, V. Nagineni, N. Gianino, J. Gu, H. Zhao, J. L. Perez-Gracia, M. F. Sanmamed, I. Melero, Elevated serum interleukin-8 is associated with enhanced intratumor neutrophils and reduced clinical benefit of immune-checkpoint inhibitors. Nat Med 26, 688-692 (2020).
31. H. Alshetaiwi, N. Pervolarakis, L. L. McIntyre, D. Ma, Q. Nguyen, J. A. Rath, K. Nee, G. Hernandez, K. Evans, L. Torosian, A. Silva, C. Walsh, K. Kessenbrock, Defining the emergence of myeloid-derived suppressor cells in breast cancer using single-cell transcriptomics. Sci Immunol 5, (2020).
32. B. Gok Yavuz, G. Gunaydin, M. E. Gedik, K. Kosemehmetoglu, D. Karakoc, F. Ozgur, D. Guc, Cancer associated fibroblasts sculpt tumour microenvironment by recruiting monocytes and inducing immunosuppressive PD-1(+) TAMs. Sci Rep 9, 3172 (2019).
33. M. Terme, E. Ullrich, L. Aymeric, K. Meinhardt, M. Desbois, N. Delahaye, S. Viaud, B.

Ryffel, H. Yagita, G. Kaplanski, A. Prevost-Blondel, M. Kato, J. L. Schultze, E. Tartour, G. Kroemer, N. Chaput, L. Zitvogel, IL-18 induces PD-1-dependent immunosuppression in cancer. Cancer Res 71, 5393-5399 (2011).
34. J. L. Benci, B. Xu, Y. Qiu, T. J. Wu, H. Dada, C. Twyman-Saint Victor, L. Cucolo, D. S. M. Lee, K. E. Pauken, A. C. Huang, T. C. Gangadhar, R. K. Amaravadi, L. M. Schuchter, M. D. Feldman, H. Ishwaran, R. H. Vonderheide, A. Maity, E. J. Wherry, A. J. Minn, Tumor Interferon Signaling Regulates a Multigenic Resistance Program to Immune Checkpoint Blockade. Cell 167, 1540-1554 e1512 (2016).
35. M. Danilo, V. Chennupati, J. G. Silva, S. Siegert, W. Held, Suppression of Tcf1 by Inflammatory Cytokines Facilitates Effector CD8 T Cell Differentiation. Cell Rep 22, 2107-2117 (2018).
36. H. K. Koblish, C. A. Hunter, M. Wysocka, G. Trinchieri, W. M. Lee, Immune suppression by recombinant interleukin (rIL)-12 involves interferon gamma induction of nitric oxide synthase 2 (iNOS) activity: inhibitors of NO generation reveal the extent of rIL-12 vaccine adjuvant effect. J Exp Med 188, 1603-1610 (1998).
37. M. F. Sanmamed, J. L. Perez-Gracia, K. A. Schalper, J. P. Fusco, A. Gonzalez, M. E. Rodriguez-Ruiz, C. Onate, G. Perez, C. Alfaro, S. Martin-Algarra, M. P. Andueza, A. Gurpide, M. Morgado, J. Wang, A. Bacchiocchi, R. Halaban, H. Kluger, L. Chen, M. Sznol, I. Melero, Changes in serum interleukin-8 (IL-8) levels reflect and predict response to anti-PD-1 treatment in melanoma and non-small-cell lung cancer patients. Ann Oncol 28, 1988-1995 (2017).
38. P. Vernocchi, F. Del Chierico, L. Putignani, Gut Microbiota Profiling: Metabolomics Based Approach to Unravel Compounds Affecting Human Health. Front Microbiol 7, 1144 (2016).
39. T. Pallister, M. A. Jackson, T. C. Martin, J. Zierer, A. Jennings, R. P. Mohney, A. MacGregor, C. J. Steves, A. Cassidy, T. D. Spector, C. Menni, Hippurate as a metabolomic marker of gut microbiome diversity: Modulation by diet and relationship to metabolic syndrome. Sci Rep 7, 13670 (2017).
40. K. Martinez-Guryn, N. Hubert, K. Frazier, S. Urlass, M. W. Musch, P. Ojeda, J. F. Pierre, J. Miyoshi, T. J. Sontag, C. M. Cham, C. A. Reardon, V. Leone, E. B. Chang, Small Intestine Microbiota Regulate Host Digestive and Absorptive Adaptive Responses to Dietary Lipids. Cell Host Microbe 23, 458-469 e455 (2018).
41. R. R. Rodrigues, N. Shulzhenko, A. Morgun, Transkingdom Networks: A Systems Biology Approach to Identify Causal Members of Host-Microbiota Interactions. Methods Mol Biol 1849, 227-242 (2018).
42. A. Yambartsev, M. A. Perlin, Y. Kovchegov, N. Shulzhenko, K. L. Mine, X. Dong, A. Morgun, Unexpected links reflect the noise in networks. Biol Direct 11, 52 (2016).
43. P. Shannon, A. Markiel, 0. Ozier, N. S. Baliga, J. T. Wang, D. Ramage, N. Amin, B. Schwikowski, T. Ideker, Cytoscape: a software environment for integrated models of biomolecular interaction networks. Genome Res 13, 2498-2504 (2003).
44. A. M. Bolger, M. Lohse, B. Usadel, Trimmomatic: a flexible trimmer for Illumina sequence data. Bioinformatics 30, 2114-2120 (2014).
45. B. Langmead, S. L. Salzberg, Fast gapped-read alignment with Bowtie 2. Nat Methods 9, 357-359 (2012).
46. D. Li, C. M. Liu, R. Luo, K. Sadakane, T. W. Lam, MEGAHIT: an ultra-fast single-node solution for large and complex metagenomics assembly via succinct de Bruijn graph. Bioinformatics 31, 1674-1676 (2015).
47. D. E. Wood, J. Lu, B. Langmead, Improved metagenomic analysis with Kraken 2. Genome Biol 20, 257 (2019).
48. T. Seemann, Prokka: rapid prokaryotic genome annotation. Bioinformatics 30, 2068-2069 (2014).
49. Z. Gu, R. Eils, M. Schlesner, Complex heatmaps reveal patterns and correlations in multidimensional genomic data. Bioinformatics 32, 2847-2849 (2016).
50. N. Segata, J. Izard, L. Waldron, D. Gevers, L. Miropolsky, W. S. Garrett, C. Huttenhower, Metagenomic biomarker discovery and explanation. Genome Biol 12, R60 (2011).
51. G. D. Bader, C. W. Hogue, An automated method for finding molecular complexes in large protein interaction networks. BMC Bioinformatics 4, 2 (2003).

1. A method of treating a cancer in a subject comprising administering to the subject a therapeutically effective amount of a fecal sample and an anti-PD-1 antibody, wherein the fecal sample is derived from a donor that is responsive to a same or different anti-PD-1 antibody.

2. The method of claim 1, wherein the cancer is a melanoma.

3. The method of claim 1, wherein the cancer is a metastatic melanoma.

4. The method of claim 2, wherein the melanoma is a PD-1 refractory melanoma.

5. The method of claim 1, wherein the subject is less responsive to the anti-PD-1 antibody than the donor prior to the administration.

6. The method of claim 1, wherein the subject is non-responsive to the anti-PD-1 antibody prior to the administration.

7. The method of claim 1, wherein the donor is responsive to a combination of the anti-PD-1 antibody and an anti-CTLA-4 antibody.

8. The method of claim 7, wherein the donor has been diagnosed with a melanoma.

9. The method of claim 8, wherein the donor has had a progression-free survival (PFS) of at least about 36 months.

10. The method of claim 1, wherein the fecal sample comprises a higher level of bacteria of phylum Actinobacteria and/or phylum Firmicutes in comparison to a control.

11. The method of claim 10, wherein the bacteria of phylum Firmicutes is a Lachnospiraceae and/or a Ruminococcaceae.

12. The method of claim 10, wherein the bacteria of phylum Actinobacteria is a Bifidobacteriaceae and/or a Coriobacteriaceae.

13. The method of claim 1, wherein the fecal sample is administered once.

14. The method of claim 1, further comprising one or more additional administrations of the anti-PD-1 antibody to the subject.

15. The method of claim 1, wherein the anti-PD-1 antibody is pembrolizumab or nivolumab.

16. The method of claim 1, wherein the method increases a level of bacteria of phylum Firmicutes and/or phylum Actinobacteria in the subject's gut in comparison to a control.

17. The method of claim 1, wherein the method decreases a level of bacteria of phylum Bacteroides in the subject's gut in comparison to a control.
